(19) United States
(12) Patent Application Publication
(10) Pub. No.: US 2005/0095607 A1
(43) Pub. Date: May 5, 2005

(54) Breast cancer signatures

(75) Inventors: Mark Erlander, Encinitas, CA (US); Xiao-Jun Ma, San Diego, CA (US); Wei Wang, San Marcos, CA (US); James L. Wittliff, Louisville, KY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arcturus Bioscience, Inc. University of Louisville

(21) Appl. No.: 10/795,092

(22) Filed: Mar. 5, 2004

Related U.S. Application Data
(60) Provisional application No. 60/453,006, filed on Mar. 7, 2003.

(51) Int. Cl. C12Q 1/68

(52) U.S. Cl. 435/6

(57) ABSTRACT

The invention relates to the identification and use of gene expression profiles, or patterns, suitable for identification of breast cancer patient populations with different survival outcomes. The gene expression profiles may be embodied in nucleic acid expression, protein expression, or other expression formats, and may be used in the study and/or determination of the prognosis of a patient, including breast cancer survival.

### RELATED APPLICATIONS

This application claims benefit of priority from U.S. Provisional Patent application No. 60/453,006, filed Mar. 7, 2003, which is hereby incorporated by reference in its entirety as if fully set forth.

### FIELD OF THE INVENTION

The invention relates to the identification and use of gene expression profiles, or patterns; with clinical relevance to breast cancer. In particular, the invention provides the identities of genes that are correlated with breast cancer recurrence, cancer metastasis, and patient survival. The gene expression profiles, whether embodied in nucleic acid expression, protein expression, or other expression formats, may be used to predict breast cancer recurrence and survival of subjects afflicted with breast cancer. The profiles may also be used in the study and/or diagnosis of breast cancer cells and tissue as well as for the study and/or determination of prognosis of a patient. When used for diagnosis or prognosis, the profiles are used to determine the treatment of breast cancer based upon the likelihood of recurrence, metastases, and life expectancy.

### BACKGROUND OF THE INVENTION

Breast cancer is by far the most common cancer among women. Each year, more than 180,000 and 1 million women in the U.S. and worldwide, respectively, are diagnosed with breast cancer. Breast cancer is the leading cause of death for women between ages 50-55, and is the most common non-preventable malignancy in women in the Western Hemisphere. An estimated 2,167,000 women in the United States are currently living with the disease (National Cancer Institute, Surveillance Epidemiology and End Results (NCI SEER) program, Cancer Statistics Review (CSR), www-seer.ims.nci.nih.gov/Publications/CSR1973 (1998)). Based on cancer rates from 1995 through 1997, a report from the National Cancer Institute (NCI) estimates that about 1 in 8 women in the United States (approximately 12.8 percent) will develop breast cancer during her lifetime (NCI's Surveillance, Epidemiology, and End Results Program (SEER) publication SEER Cancer Statistics Review 1973-1997). Breast cancer is the second most common form of cancer, after skin cancer, among women in the United States. An estimated 250,100 new cases of breast cancer are expected to be diagnosed in the United States in 2001. Of these, 192,200 new cases of more advanced (invasive) breast cancer are expected to occur among women (an increase of 5% over last year), 46,400 new cases of early stage (in situ) breast cancer are expected to occur among women (up 9% from last year), and about 1,500 new cases of breast cancer are expected to be diagnosed in men (Cancer Facts & Figures 2001 American Cancer Society). An estimated 40,600 deaths (40,300 women, 400 men) from breast cancer are expected in 2001. Breast cancer ranks second only to lung cancer among causes of cancer deaths in women. Nearly 86% of women who are diagnosed with breast cancer are likely to still be alive five years later, though 24% of them will die of breast cancer after 10 years, and nearly half (47%) will die of breast cancer after 20 years.

Every woman is at risk for breast cancer. Over 70 percent of breast cancers occur in women who have no identifiable risk factors other than age (U.S. General Accounting Office. Breast Cancer, 1971-1991: Prevention, Treatment and Research. GAO/PEMD-92-12; 1991). Only 5 to 10% of breast cancers are linked to a family history of breast cancer (Henderson IC, Breast Cancer. In: Murphy G P, Lawrence W L, Lenhard R E (eds). Clinical Oncology. Atlanta, Ga.: American Cancer Society; 1995:198-219).

Each breast has 15 to 20 sections called lobes. Within each lobe are many smaller lobules. Lobules end in dozens of tiny bulbs that can produce milk. The lobes, lobules, and bulbs are all linked by thin tubes called ducts. These ducts lead to the nipple in the center of a dark area of skin called the areola. Fat surrounds the lobules and ducts. There are no muscles in the breast, but muscles lie under each breast and cover the ribs. Each breast also contains blood vessels and lymph vessels. The lymph vessels carry colorless fluid called lymph, and lead to the lymph nodes. Clusters of lymph nodes are found near the breast in the axilla (under the arm), above the collarbone, and in the chest.

Breast tumors can be either benign or malignant. Benign tumors are not cancerous, they do not spread to other parts of the body, and are not a threat to life. They can usually be removed, and in most cases, do not come back. Malignant tumors are cancerous, and can invade and damage nearby tissues and organs. Malignant tumor cells may metastasize, entering the bloodstream or lymphatic system. When breast cancer cells metastasize outside the breast, they are often found in the lymph nodes under the arm (axillary lymph nodes). If the cancer has reached these nodes, it means that cancer cells may have spread to other lymph nodes or other organs, such as bones, liver, or lungs.

Major and intensive research has been focussed on early detection, treatment and prevention. This has included an emphasis on determining the presence of precancerous or cancerous ductal epithelial cells. These cells are analyzed, for example, for cell morphology, for protein markers, for nucleic acid markers, for chromosomal abnormalities, for biochemical markers, and for other characteristic changes that would signal the presence of cancerous or precancerous cells. This has led to various molecular alterations that have been reported in breast cancer, few of which have been well characterized in human clinical breast specimens. Molecular alterations include presence/absence of estrogen and progesterone steroid receptors, HER-2 expression/amplification (Mark H F, et al. HER-2/neu gene amplification in stages I-IV breast cancer detected by fluorescent in situ hybridization. Genet Med; 1(3):98-103 1999), Ki-67 (an antigen that is present in all stages of the cell cycle except G0 and used as a marker for tumor cell proliferation, and prognostic markers (including oncogenes, tumor suppressor genes, and angiogenesis markers) like p53, p27, Cathepsin D, pS2, multi-drug resistance (MDR) gene, and CD31.

van't Veer et al. (Nature 415:530-536, 2002) describe gene expression profiling of clinical outcome in breast cancer. They identified genes expressed in breast cancer tumors, the expression levels of which correlated either with patients afflicted with distant metastases within 5 years or with patients that remained metastasis-free after at least 5 years.

Ramaswamy et al. (Nature Genetics 33:49-54, 2003) describe the identification of a molecular signature of metastasis in primary solid tumors. The genes of the signature were identified based on gene expression profiles of 12 metastatic adenocarcinoma nodules of diverse origin (lung, breast, prostate, colorectal, uterus) compared to expression profiles of 64 primary adenocarcinomas representing the same spectrum of tumor types from different individuals. A 128 gene set was identified.

Both of the above described approaches, however, utilize heterogeneous populations of cells found in a tumor sample to obtain information on gene expression patterns. The use of such populations may result in the inclusion or exclusion of multiple genes that are differentially expressed in cancer cells. The gene expression patterns observed by the above described approaches may thus provide little confidence that the differences in gene expression are meaningfully associated with breast cancer recurrence or survival.

Citation of documents herein is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

The present invention relates to the identification and use of gene expression patterns (or profiles or “signatures”) which are clinically relevant to breast cancer. In particular, the identities of genes that are correlated with breast cancer recurrence, cancer metastasis, and patient survival are provided. The gene expression profiles, whether embodied in nucleic acid expression, protein expression, or other expression formats, may be used to predict breast cancer recurrence and survival of subjects afflicted with breast cancer.

The invention thus provides for the identification and use of gene expression patterns (or profiles or “signatures”) which correlate with (and thus able to discriminate between) patients with good or poor survival outcomes. In one embodiment, the invention provides patterns that are able to distinguish patients with estrogen receptor (ER) positive breast tumors into those with poor survival outcomes, similar to that of patients with ER negative breast tumors, and those with a better survival outcome. These patterns are thus able to distinguish patients with ER positive breast tumors into at least two subtypes. Other patterns are capable of identifying subjects with ER negative tumors, and the survival outcomes associated therewith, as well as survival outcomes for some breast cancer subjects independent of the ER status of their tumors.

The invention also provides for the identification and use of gene expression patterns which correlate with the recurrence of breast cancer in the form of metastases. The patterns are able to distinguish patients with breast cancer into at least those with good or poor survival outcomes.

The present invention provides a non-subjective means for the identification of patients with breast cancer as likely to have a good or poor survival outcome by assaying for the expression patterns disclosed herein. Thus where subjective interpretation may have been previously used to determine the prognosis and/or treatment of breast cancer patients, the present invention provides objective gene expression patterns, which may used alone or in combination with subjective criteria to provide a more accurate assessment of breast cancer patient outcomes. The expression patterns of the invention thus provide a means to determine breast cancer prognosis. Furthermore, the expression patterns can also be used as a means to assay small, node negative tumors that are not readily assayed by other means.

The gene expression patterns comprise one or more than one gene capable of discriminating between breast cancer survival outcomes with significant accuracy. The gene(s) are identified as correlated with various breast cancer survival outcomes such that the levels of their expression are relevant to a determination of the survival, and thus preferred treatment protocols, of a breast cancer patient. Thus in one aspect, the invention provides a method to determine the survival outcome of a subject afflicted with, or suspected of having, breast cancer by assaying a cell containing sample from said subject for expression of one or more than one gene disclosed herein as correlated with breast cancer survival outcomes.

Gene expression patterns of the invention are identified as described below. Generally, a large sampling of gene expression profile of a sample is obtained through quantifying the expression levels of mRNA corresponding to many genes. This profile is then analyzed to identify genes, the expression of which are positively, or negatively, correlated, with breast cancer survival outcomes. An expression profile of a subset of human genes may then be identified by the methods of the present invention as correlated with a particular breast cancer survival outcome. The use of multiple samples increases the confidence which a gene may be believed to be correlated with a particular survival outcome. Without sufficient confidence, it remains unpredictable whether a particular gene is actually correlated with breast cancer survival outcomes and also unpredictable whether a particular gene may be successfully used to identify the survival outcome for a breast cancer patient.

A profile of genes that are highly correlated with one survival outcome relative to another may be used to assay an sample from a subject afflicted with, or suspected of having, breast cancer to predict the survival outcome of the subject from whom the sample was obtained. Such an assay may be used as part of a method to determine the therapeutic treatment for said subject based upon the breast cancer survival outcome identified.

The correlated genes may be used singly with significant accuracy or in combination to increase the ability to accurately discriminate between various stages and/or grades of breast cancer. The present invention thus provides means for correlating a molecular expression phenotype with breast cancer survival outcomes. This correlation is a way to molecularly provide for the determine survival outcomes as disclosed herein. Additional uses of the correlated gene(s) are in the classification of cells and tissues; determination of diagnosis and/or prognosis; and determination and/or alteration of therapy.

An assay of the invention may utilize a means related to the expression level of the sequences disclosed herein as long as the assay reflects, quantitatively or qualitatively, expression of the sequence. Preferably, however, a quantitative assay means is preferred. The ability to discriminate is conferred by the identification of expression of the individual genes as relevant and not by the form of the assay used to determine the actual level of expression. An assay may utilize any identifying feature of an identified individual gene as disclosed herein as long as the assay reflects, quantitatively or qualitatively, expression of the gene. Identifying features include, but are not limited to, unique nucleic acid sequences used to encode (DNA), or express (RNA), said gene or epitopes specific to, or activities of, a protein encoded by said gene. Alternative means include detection of nucleic acid amplification as indicative of increased expression levels and nucleic acid inactivation, deletion, or methylation, as indicative of decreased expression levels. Stated differently, the invention may be practiced by assaying one or more aspect of the DNA template(s) underlying the expression of the disclosed sequence(s), of the RNA used as an intermediate to express the sequence(s), or of the proteinaceous product expressed by the sequence(s), as well as proteolytic fragments of such products. As such, the detection of the presence of, amount of, stability of, or degradation (including rate) of, such DNA, RNA and proteinaceous molecules may be used in the practice of the invention. As such, all that is required is the identity of the gene(s) necessary to discriminate between breast cancer survival outcomes and an appropriate cell containing sample for use in an expression assay.

In one aspect, the invention provides for the identification of the gene expression patterns by analyzing global, or near global, gene expression from single cells or homogenous cell populations which have been dissected away from, or otherwise isolated or purified from, contaminating cells beyond that possible by a simple biopsy. Because the expression of numerous genes fluctuate between cells from different patients as well as between cells from the same patient sample, multiple data from expression of individual genes and gene expression patterns are used as reference data to generate models which in turn permit the identification of individual gene(s), the expression of which are most highly correlated with particular breast cancer survival outcomes.

In a further aspect, the gene sequence(s) capable of discriminating between breast cancer survival outcomes based on cell or tissue samples may be used to determine the likely outcome of a patient from whom the sample was obtained. Preferably, the sample is isolated via non-invasive means. The expression of said gene(s) in said sample may be determined and compared to the expression of said gene(s) in reference data of gene expression patterns as disclosed herein. Alternatively, the expression level may be compared to expression levels in normal or non-cancerous cells, such as, but not limited to, those from the same sample or subject. In embodiments of the invention utilizing quantitative PCR, the expression level may be compared to expression levels of reference genes in the same sample or a ratio of expression levels may be used. The invention provides for ratios of the expression level of a sequence that is underexpressed to the expression level of a sequence that is overexpressed as a indicator of survival outcome or cancer recurrence, including metastatic cancer. The use of a ratio can reduce comparisons with normal or non-cancerous cells.

One advantage provided by the present invention is that contaminating, non-breast cells (such as infiltrating lymphocytes or other immune system cells) are not present to possibly affect the genes identified or the subsequent analysis of gene expression to identify the survival outcomes of patients with breast cancer. Such contamination is present where a biopsy is used to generate gene expression profiles.

While the present invention has been described mainly in the context of human breast cancer, it may be practiced in the context of breast cancer of any animal known to be potentially afflicted by breast cancer. Preferred animals for the application of the present invention are mammals, particularly those important to agricultural applications (such as, but not limited to, cattle, sheep, horses, and other “farm animals”) and for human companionship (such as, but not limited to, dogs and cats).

### BRIEF DESCRIPTION OF THE DRAWINGS

FIG. 1 is a clinical outcome (overall survival) plot of two subtypes based on expression of 864 genes as listed in Tables 2 and 3.

FIG. 2 is a plot of a 297 gene signature (identities of the genes are presented in Table 5) which segregates the survival data of a patient population into “long” and “short” groups with significantly different overall survival curves. FIG. 2 also shows the comparison of this 297 gene set with that of a set of 17 genes correlated with matastasis described by Ramaswamy et al. (supra, see Table 1 therein).

FIG. 3 is a plot of clinical outcomes for four breast cancer subtypes provided by the instant invention.

### DETAILED DESCRIPTION OF THE SPECIFIC EMBODIMENTS

Definitions of Terms as used Herein:

A gene expression “pattern” or “profile” or “signature” refers to the relative expression of a gene between two or more breast cancer survival outcomes which is correlated with being able to distinguish between said outcomes.

A “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “sequence” or “gene sequence” as used herein is a nucleic acid molecule or polynucleotide composed of a discrete order of nucleotide bases. The term includes the ordering of bases that encodes a discrete product (i.e. “coding region”), whether RNA or proteinaceous in nature, as well as the ordered bases that precede or follow a “coding region”. Non-limiting examples of the latter include 5′ and 3′ untranslated regions of a gene. It is appreciated that more than one polynucleotide may be capable of encoding a discrete product. It is also appreciated that alleles and polymorphisms of the disclosed sequences may exist and may be used in the practice of the invention to identify the expression level(s) of the disclosed sequences or the allele or polymorphism. Identification of an allele or polymorphism depends in part upon chromosomal location and ability to recombine during mitosis.

The terms “correlate” or “correlation” or equivalents thereof refer to an association between expression of one or more genes in a breast cancer cell or tissue sample and the survival outcome of the subject from whom the sample was obtained. Genes expressed at higher levels and correlated with the survival outcomes disclosed herein are provided. The invention provides for the correlation between increases, as well as decreases, in expression of gene sequences and survival outcomes and cancer recurrence, including cancer metastases, in patients. Increases and decreases may be readily expressed in the form of a ratio between expression in a non-normal cell and a normal cell such that a ratio of one (1) indicates no difference while ratios of two (2) and one-half indicate twice as much, and half as much, expression in the non-normal cell versus the normal cell, respectively. Expression levels can be readily determined by quantitative methods as described below.

For example, increases in gene expression can be indicated by ratios of or about 1.1, of or about 1.2, of or about 1.3, of or about 1.4, of or about 1.5, of or about 1.6, of or about 1.7, of or about 1.8, of or about 1.9, of or about 2, of or about 2.5, of or about 3, of or about 3.5, of or about 4, of or about 4.5, of or about 5, of or about 5.5, of or about 6, of or about 6.5, of or about 7, of or about 7.5, of or about 8, of or about 8.5, of or about 9, of or about 9.5, of or about 10, of or about 15, of or about 20, of or about 30, of or about 40, of or about 50, of or about 60, of or about 70, of or about 80, of or about 90, of or about 100, of or about 150, of or about 200, of or about 300, of or about 400, of or about 500, of or about 600, of or about 700, of or about 800, of or about 900, or of or about 1000. A ratio of 2 is a 100% (or a two-fold) increase in expression. Decreases in gene expression can be indicated by ratios of or about 0.9, of or about 0.8, of or about 0.7, of or about 0.6, of or about 0.5, of or about 0.4, of or about 0.3, of or about 0.2, of or about 0.1, of or about 0.05, of or about 0.01, of or about 0.005, of or about 0.001, of or about 0.0005, of or about 0.0001, of or about 0.00005, of or about 0.00001, of or about 0.000005, or of or about 0.000001.

In some embodiments of the invention, such as those related to survival, cancer recurrence, or metastasis as possible outcome phenotypes, a ratio of the expression of a gene sequence expressed at increased levels in correlation with an outcome to the expression of a gene sequence expressed at decreased levels in correlation with the outcome may also be used as an indicator of the phenotype. As a non-limiting example, one cancer survival outcome may be correlated with increased expression of a gene sequence overexpressed in a sample of cancer cells as well as decreased expression of another gene sequence underexpressed in those cells. Therefore, a ratio of the expression levels of the underexpressed sequence to the expression levels of the overexpressed sequence may be used as an indicator or predictor of the ourcome.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and intemucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence.

By corresponding is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17). Methods for amplifying mRNA are generally known in the art, and include reverse transcription PCR (RT-PCR) and those described in U.S. patent application Ser. No. 10/062,857 (filed on Oct. 25, 2001), as well as U.S. Provisional Patent Applications 60/298,847 (filed Jun. 15, 2001) and 60/257,801 (filed Dec. 22, 2000), all of which are hereby incorporated by reference in their entireties as if fully set forth. Another method which may be used is quantitative PCR (or Q-PCR). Alternatively, RNA may be directly labeled as the corresponding cDNA by methods known in the art.

A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². Preferably, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of primers in the array is known, the identities of a sample polynucleotides can be determined based on their binding to a particular position in the microarray.

Because the invention relies upon the identification of genes that are over- or under-expressed, one embodiment of the invention involves determining expression by hybridization of mRNA, or an amplified or cloned version thereof, of a sample cell to a polynucleotide that is unique to a particular gene sequence. Preferred polynucleotides of this type contain at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Even more preferred are polynucleotides of at least or about 50, at least or about 100, at least about or 150, at least or about 200, at least or about 250, at least or about 300, at least or about 350, or at least or about 400 basepairs of a gene sequence that is not found in other gene sequences. The term “about” as used in the preceding sentence refers to an increase or decrease of 10% from the stated numerical value. Such polynucleotides may also be referred to as polynucleotide probes that are capable of hybridizing to sequences of the genes, or unique portions thereof, described herein. Preferably, the sequences are those of mRNA encoded by the genes, the corresponding cDNA to such mRNAs, and/or amplified versions of such sequences. In preferred embodiments of the invention, the polynucleotide probes are immobilized on an array, other devices, or in individual spots that localize the probes.

In another embodiment of the invention, all or part of a disclosed sequence may be amplified and detected by methods such as the polymerase chain reaction (PCR) and variations thereof, such as, but not limited to, quantitative PCR (Q-PCR), reverse transcription PCR (RT-PCR), and real-time PCR (including as a means of measuring the initial amounts of mRNA copies for each sequence in a sample), optionally real-time RT-PCR or real-time Q-PCR. Such methods would utilize one or two primers that are complementary to portions of a disclosed sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization. Additional methods to detect the expression of expressed nucleic acids include RNAse protection assays, including liquid phase hybridizations, and in situ hybridization of cells.

Alternatively, and in yet another embodiment of the invention, gene expression may be determined by analysis of expressed protein in a cell sample of interest by use of one or more antibodies specific for one or more epitopes of individual gene products (proteins), or proteolytic fragments thereof, in said cell sample or in a bodily fluid of a subject. The cell sample may be one of breast cancer epithelial cells enriched from the blood of a subject, such as by use of labeled antibodies against cell surface markers followed by fluorescence activated cell sorting (FACS). Such antibodies are preferably labeled to permit their easy detection after binding to the gene product. Detection methodologies suitable for use in the practice of the invention include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR.

The term “label” refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

As used herein, a “breast tissue sample” or “breast cell sample” refers to a sample of breast tissue or fluid isolated from an individual suspected of being afflicted with, or at risk of developing, breast cancer. Such samples are primary isolates (in contrast to cultured cells) and may be collected by any non-invasive means, including, but not limited to, ductal lavage, fine needle aspiration, needle biopsy, the devices and methods described in U.S. Pat. No. 6,328,709, or any other suitable means recognized in the art. Alternatively, the “sample” may be collected by an invasive method, including, but not limited to, surgical biopsy. A sample of the invention may also be one that has been formalin fixed and paraffin embedded (FFPE) or freshly frozened.

“Expression” and “gene expression” include transcription and/or translation of nucleic acid material.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion or insertion. Single nucleotide polymorphism (SNP) is also a sequence mutation as used herein. Because the present invention is based on the relative level of gene expression, mutations in non-coding regions of genes as disclosed herein may also be assayed in the practice of the invention.

“Detection” includes any means of detecting, including direct and indirect detection of gene expression and changes therein. For example, “detectably less” products may be observed directly or indirectly, and the term indicates any reduction (including the absence of detectable signal). Similarly, “detectably more” product means any increase, whether observed directly or indirectly.

Increases and decreases in expression of the disclosed sequences are defined in the following terms based upon percent or fold changes over expression in normal cells. Increases may be of 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 10, 20, 30, 40, 50, 55, 60, 65, 70, 75, 80, 82, 84, 86, 88, 90, 92, 94, 96, 98, 99 or 100% relative to expression levels in normal cells.

Unless defined otherwise all technical and scientific terms used herein have the same meaning as commonly understood to one of ordinary skill in the art to which this invention belongs.

Specific Embodiments

The present invention relates to the identification and use of gene expression patterns (or profiles or “signatures”) which discriminate between (or are correlated with) breast cancer survival outcomes in a subject. Such patterns may be determined by the methods of the invention by use of a number of reference cell or tissue samples, such as those reviewed by a pathologist of ordinary skill in the pathology of breast cancer, which reflect breast cancer cells as opposed to normal or other non-cancerous cells. Because the overall gene expression profile differs from person to person, cancer to cancer, and cancer cell to cancer cell, correlations between certain cells and overexpressed genes may be made as disclosed herein to identify genes that are capable of discriminating between breast cancer survival outcomes.

The present invention may be practiced with any number of the genes believed, or likely to be, differentially expressed with respect to breast cancer survival outcomes. The identification may be made by using expression profiles of various homogenous breast cancer cell populations, which were isolated by microdissection, such as, but not limited to, laser capture microdissection (LCM) of 100-1000 cells. The expression level of each gene of the expression profile may be correlated with a particular survival outcome. Alternatively, the expression levels of multiple genes may be clustered to identify correlations with particular survival outcomes.

Genes with significant correlations to breast cancer survival outcomes may be used to generate models of gene expressions that would maximally discriminate between survival outcomes. Alternatively, genes with significant correlations may be used in combination with genes with lower correlations without significant loss of ability to discriminate between survival outcomes. Such models may be generated by any appropriate means recognized in the art, including, but not limited to, cluster analysis, supported vector machines, neural networks or other algorithm known in the art. The models are capable of predicting the classification of a unknown sample based upon the expression of the genes used for discrimination in the models. “Leave one out” cross-validation may be used to test the performance of various models and to help identify weights (genes) that are uninformative or detrimental to the predictive ability of the models. Cross-validation may also be used to identify genes that enhance the predictive ability of the models.

The gene(s) identified as correlated with particular breast cancer survival outcomes by the above models provide the ability to focus gene expression analysis to only those genes that contribute to the ability to identify a subject as likely to have a particular survival outcome relative to another. The expression of other genes in a breast cancer cell would be relatively unable to provide information concerning, and thus assist in the discrimination of, breast cancer survival outcome.

As will be appreciated by those skilled in the art, the models are highly useful with even a small set of reference gene expression data and can become increasingly accurate with the inclusion of more reference data although the incremental increase in accuracy will likely diminish with each additional datum. The preparation of additional reference gene expression data using genes identified and disclosed herein for discriminating between different survival outcomes in breast cancer is routine and may be readily performed by the skilled artisan to permit the generation of models as described above to predict the status of an unknown sample based upon the expression levels of those genes.

To determine the (increased or decreased) expression levels of genes in the practice of the present invention, any method known in the art may be utilized. In one preferred embodiment of the invention, expression based on detection of RNA which hybridizes to the genes identified and disclosed herein is used. This is readily performed by any RNA detection or amplification+detection method known or recognized as equivalent in the art such as, but not limited to, reverse transcription-PCR, the methods disclosed in U.S. patent application Ser. No. 10/062,857 (filed on Oct. 25, 2001) as well as U.S. Provisional Patent Applications No. 60/298,847 (filed Jun. 15, 2001) and 60/257,801 (filed Dec. 22, 2000), and methods to detect the presence, or absence, of RNA stabilizing or destabilizing sequences.

Alternatively, expression based on detection of DNA status may be used. Detection of the DNA of an identified gene as methylated or deleted may be used for genes that have decreased expression in correlation with survival outcomes. This may be readily performed by, PCR based methods known in the art, including, but not limited to, quantitative PCR (Q-PCR). Conversely, detection of the DNA of an identified gene as amplified may be used for genes that have increased expression in correlation with survival outcomes. This may be readily performed by PCR based, fluorescent in situ hybridization (FISH) and chromosome in situ hybridization (CISH) methods known in the art.

Expression based on detection of a presence, increase, or decrease in protein levels or activity may also be used. Detection may be performed by any immunohistochemistry (IHC) based, blood based (especially for secreted proteins), antibody (including autoantibodies against the protein) based, ex foliate cell (from the cancer) based, mass spectroscopy based, and image (including used of labeled ligand) based method known in the art and recognized as appropriate for the detection of the protein. Antibody and image based methods are additionally useful for the localization of tumors after determination of cancer by use of cells obtained by a non-invasive procedure (such as ductal lavage or fine needle aspiration), where the source of the cancerous cells is not known. A labeled antibody or ligand may be used to localize the carcinoma(s) within a patient.

A preferred embodiment using a nucleic acid based assay to determine expression is by immobilization of one or more sequences of the genes identified herein on a solid support, including, but not limited to, a solid substrate as an array or to beads or bead based technology as known in the art. Alternatively, solution based expression assays known in the art may also be used. The immobilized gene(s) may be in the form of polynucleotides that are unique or otherwise specific to the gene(s) such that the polynucleotide would be capable of hybridizing to a DNA or RNA corresponding to the gene(s). These polynucleotides may be the full length of the gene(s) or be short sequences of the genes (up to one nucleotide shorter than the full length sequence known in the art by deletion from the 5′ or 3′ end of the sequence) that are optionally minimally interrupted (such as by mismatches or inserted non-complementary basepairs) such that hybridization with a DNA or RNA corresponding to the gene(s) is not affected. Preferably, the polynucleotides used are from the 3′ end of the gene, such as within about 350, about 300, about 250, about 200, about 150, about 100, or about 50 nucleotides from the polyadenylation signal or polyadenylation site of a gene or expressed sequence. Polynucleotides containing mutations relative to the sequences of the disclosed genes may also be used so long as the presence of the mutations still allows hybridization to produce a detectable signal.

Alternatively, amplification of such sequences from the 3′ end of genes by methods such as quantitative PCR may be used to determine the expression levels of the sequences. The Ct values generated by such methods may be used as indicators of expression levels.

The immobilized gene(s) may be used to determine the state of nucleic acid samples prepared from sample breast cell(s) for which the survival outcome of the sample's subject (e.g. patient from whom the sample is obtained) is not known or for confirmation of an outcome that is already assigned to the sample's subject. Without limiting the invention, such a cell may be from a patient suspected of being afflicted with, or at risk of developing, breast cancer. The immobilized polynucleotide(s) need only be sufficient to specifically hybridize to the corresponding nucleic acid molecules derived from the sample. While even a single correlated gene sequence may to able to provide adequate accuracy in discriminating between two breast cancer survival outcomes, two or more, three or more, four or more, five or more, six or more, seven or more, eight or more, nine or more, ten or more, or eleven or more of the genes identified herein may be used as a subset capable of discriminating may be used in combination to increase the accuracy of the method. The invention specifically contemplates the selection of more than one, two or more, three or more, four or more, five or more, six or more, seven or more, eight or more, nine or more, ten or more, or eleven or more of the genes disclosed in the tables and figures herein for use as a subset in the identification of breast cancer survival outcome.

Of course 15 or more, 20 or more, 30 or more, 40 or more, 50 or more, 60 or more, 70 or more, 80 or more, 90 or more, 100 or more, 150 or more, 200 or more, 250 or more, 300 or more, 350 or more, 400 or more, 450 or more, 500 or more, 600 or more, 700 or more, 800 or more, 900 or more, 1000 or more, 1100 or more, 1200 or more, or all the genes provided in Tables 2, 3, and/or 4 below may be used. “CloneID” as used in the context of the Tables herein as well as the present invention refers to the IMAGE Consortium clone ID number of each gene, the sequences of which are hereby incorporated by reference in their entireties as they are available from the Consortium at image.llnl.gov/ as accessed on the filing date of the present application. “GeneID” as used in the context of the Tables herein as well as the present invention refers to the GenBank accession number of a sequence of each gene, the sequences of which are hereby incorporated by reference in their entireties as they are available from GenBank as accessed on the filing date of the present application.

P value refers to values assigned as described in the Example below. The indications of “E-xx” where “xx” is a two digit number refers to alternative notation for exponential figures where “E-xx” is “10^{-xx}”. Thus in combination with the numbers to the left of“E-xx”, the value being represented is the numbers to the left times 10^{-xx}. Chromosome Location refers to the human chromosome to which the gene has been assigned, and Description provides a brief identifier of what the gene encodes.

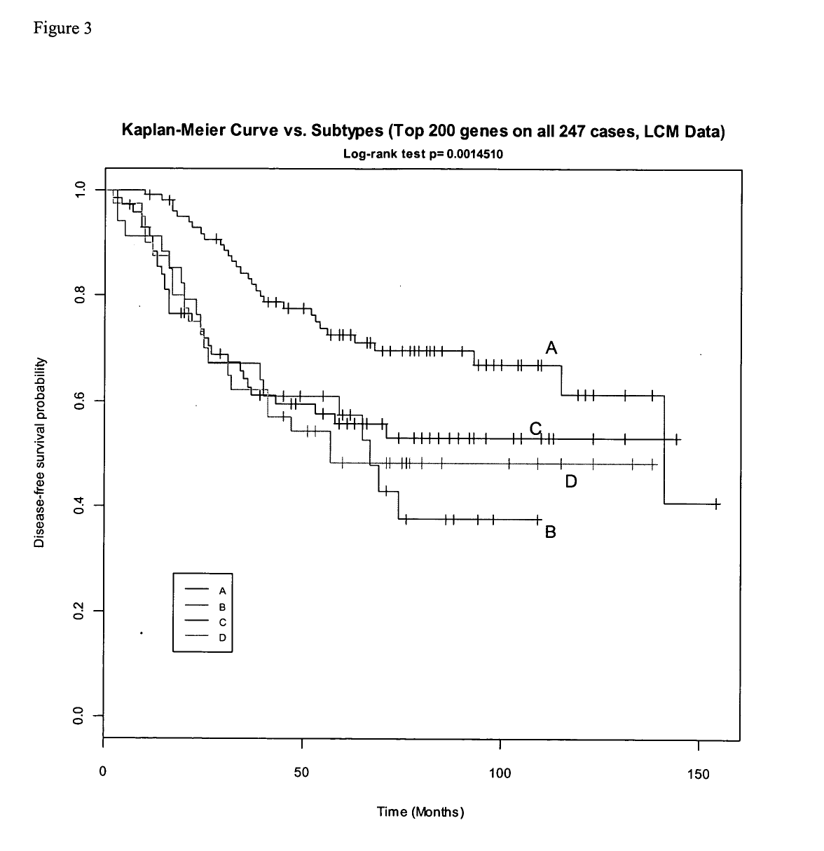

The invention may also be practiced with all or a portion of the gene sequences disclosed in Tables 6, 7, 8, and 9 herein. The gene sequences of each of these tables define one of four breast cancer subtypes based upon increased expression in correlation with particular survival outcomes as shown in FIG. 3. Therefore, the increased expression of sequences of 2 or more, 4 or more, 6 or more, 8 or more, 10 or more, 12 or more, 14 or more, 16 or more, 18 or more, 20 or more, 22 or more, 24 or more, 26 or more, 28 or more, 30 or more, 32 or more, 34 or more, 36 or more, 38 or more, 40 or more, 42 or more, 44 or more, 46 or more, 48 or more, or all 50 genes in each table can be used in the practice of the invention as indicators of a breast cancer survival outcome. Of course sequences of the 25 possible odd numbers of these genes may also be used.

Genes with a correlation identified by a p value below or about 0.02, below or about 0.01, below or about 0.005, below or about 0.001, below or about 1×10⁻⁴, below or about 1×10⁻⁵, below or about 1×10⁻⁶, below or about 1×10⁻⁷, below or about 1×10⁻⁸, below or about 1×10⁻⁹, below or about 1×10⁻¹⁰, below or about 1×10⁻¹¹, below or about 1×10⁻¹², below or about 1×10⁻¹³, below or about 1×10⁻¹⁴, below or about 1×10⁻¹⁵, below or about 1×10⁻¹⁶, below or about 1×10⁻¹⁷, below or about 1×10⁻¹⁸, below or about 1×10⁻¹⁹, or about 1×10⁻²⁰ are preferred for use in the practice of the invention. The present invention includes the use of genes that identify different ERα (estrogen receptor alpha) positive subtypes and breast cancer recurrence/metastases together to permit simultaneous identification of breast cancer survival outcome of a patient based upon assaying a breast cancer sample from said patient.

In some embodiments of the invention, the genes used will not include HRAS-like suppressor (UNIGENE ID Hs.36761; CloneID 950667; GenBank accession # NM_{—}020386; and GeneSymbol HRASLS) and/or origin recognition complex, subunit 6 (yeast homolog)-like, (UNIGENE ID Hs.49760; CloneID 306318; GenBank accession # NM_{—}014321; and GeneSymbol ORC6L) as disclosed by van't Veer et al. (supra).

In embodiments where only one or a few genes are to be analyzed, the nucleic acid derived from the sample breast cancer cell(s) may be preferentially amplified by use of appropriate primers such that only the genes to be analyzed are amplified to reduce contaminating background signals from other genes expressed in the breast cell. Alternatively, and where multiple genes are to be analyzed or where very few cells (or one cell) is used, the nucleic acid from the sample may be globally amplified before hybridization to the immobilized polynucleotides. Of course RNA, or the cDNA counterpart thereof may be directly labeled and used, without amplification, by methods known in the art.

The invention is preferably practiced with unique sequences present within the gene sequences disclosed herein. The uniqueness of a disclosed gene sequence refers to the portions or entireties of the sequences which are found in each gene to the exclusion of other genes. Such unique sequences include those found at the 3′ untranslated portion of the genes. Preferred unique sequences for the practice of the invention are those which contribute to the consensus sequences for each gene such that the unique sequences will be useful in detecting expression in a variety of individuals rather than being specific for a polymorphism present in some individuals. Alternatively, sequences unique to an individual or a subpopulation may be used. The preferred unique sequences are preferably of the lengths of polynucleotides of the invention as discussed herein.

In particularly preferred embodiments of the invention, polynucleotides having sequences present in the 3′ untranslated and/or non-coding regions of the disclosed gene sequences are used to detect expression levels in breast cells. Such polynucleotides may optionally contain sequences found in the 3′ portions of the coding regions of the disclosed sequences. Polynucleotides containing a combination of sequences from the coding and 3′ non-coding regions preferably have the sequences arranged contiguously, with no intervening heterologous sequence(s).

Alternatively, the invention may be practiced with polynucleotides having sequences present in the 5′ untranslated and/or non-coding regions of gene sequences in breast cells to detect their levels of expression. Such polynucleotides may optionally contain sequences found in the 5′ portions of the coding regions. Polynucleotides containing a combination of sequences from the coding and 5′ non-coding regions preferably have the sequences arranged contiguously, with no intervening heterologous sequence(s). The invention may also be practiced with sequences present in the coding regions of disclosed sequences.

Preferred polynucleotides contain sequences from 3′ or 5′ untranslated and/or non-coding regions of at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, or at least about 46 consecutive nucleotides. The term “about” as used in the previous sentence refers to an increase or decrease of 1 from the stated numerical value. Even more preferred are polynucleotides containing sequences of at least or about 50, at least or about 100, at least about or 150, at least or about 200, at least or about 250, at least or about 300, at least or about 350, or at least or about 400 consecutive nucleotides. The term “about” as used in the preceding sentence refers to an increase or decrease of 10% from the stated numerical value.

Sequences from the 3′ or 5′ end of the above described coding regions as found in polynucleotides of the invention are of the same lengths as those described above, except that they would naturally be limited by the length of the coding region. The 3′ end of a coding region may include sequences up to the 3′ half of the coding region. Conversely, the 5′ end of a coding region may include sequences up the 5′ half of the coding region. Of course the above described sequences, or the coding regions and polynucleotides containing portions thereof, may be used in their entireties.

Polynucleotides combining the sequences from a 3′ untranslated and/or non-coding region and the associated 3′ end of the coding region are preferably at least or about 100, at least about or 150, at least or about 200, at least or about 250, at least or about 300, at least or about 350, or at least or about 400 consecutive nucleotides. Preferably, the polynucleotides used are from the 3′ end of the gene, such as within about 350, about 300, about 250, about 200, about 150, about 100, or about 50 nucleotides from the polyadenylation signal or polyadenylation site of a gene or expressed sequence. Polynucleotides containing mutations relative to the sequences of the disclosed genes may also be used so long as the presence of the mutations still allows hybridization to produce a detectable signal.

In another embodiment of the invention, polynucleotides containing deletions of nucleotides from the 5′ and/or 3′ end of the above disclosed sequences may be used. The deletions are preferably of 1-5, 5-10, 10-15, 15-20, 20-25, 25-30, 30-35, 35-40, 40-45-50, 50-60, 60-70, 70-80, 80-90, 90-100, 100-125, 125-150, 150-175, or 175-200 nucleotides from the 5′ and/or 3′ end, although the extent of the deletions would naturally be limited by the length of the disclosed sequences and the need to be able to use the polynucleotides for the detection of expression levels.

Other polynucleotides of the invention from the 3′ end of the above disclosed sequences include those of primers and optional probes for quantitative PCR. Preferably, the primers and probes are those which amplify a region less than about 350, less than about 300, less than about 250, less than about 200, less than about 150, less than about 100, or less than about 50 nucleotides from the from the polyadenylation signal or polyadenylation site of a gene or expressed sequence.

In yet another embodiment of the invention, polynucleotides containing portions of the above disclosed sequences including the 3′ end may be used in the practice of the invention. Such polynucleotides would contain at least or about 50, at least or about 100, at least about or 150, at least or about 200, at least or about 250, at least or about 300, at least or about 350, or at least or about 400 consecutive nucleotides from the 3′ end of the disclosed sequences.

The above assay embodiments may be used in a number of different ways to identify or detect the breast cancer stage and/or grade, if any, of a breast cancer cell sample from a patient as well as the likely survival outcome of said patient. In many cases, this would reflect a secondary screen for the patient, who may have already undergone mammography or physical exam as a primary screen. If positive, the subsequent needle biopsy, ductal lavage, fine needle aspiration, or other analogous methods may provide the sample for use in the above assay embodiments. The present invention is particularly useful in combination with non-invasive protocols, such as ductal lavage or fine needle aspiration, to prepare a breast cell sample.

The present invention provides a more objective set of criteria, in the form of gene expression profiles of a discrete set of genes, to discriminate (or delineate) between breast cancer survival outcomes. In particularly preferred embodiments of the invention, the assays are used to discriminate between good and poor outcomes within 5, or about 5, years after surgical intervention to remove breast cancer tumors or within about 95 months after surgical intervention to remove breast cancer tumors. Comparisons that discriminate between outcomes after about 10, about 20, about 30, about 40, about 50, about 60, about 70, about 80, about 90, about 100, about 110, about 120, about 130, about 140, or about 150 months may also be performed.

While good and poor survival outcomes may be defined relatively in comparison to each other, a “good” outcome may be viewed as a better than 50% survival rate after about 60 months post surgical intervention to remove breast cancer tumor(s). A “good” outcome may also be a better than about 60%, about 70%, about 80% or about 90% survival rate after about 60 months post surgical intervention. A “poor” outcome may be viewed as an about 60% or less, or about 50% or less, survival rate after about 40 or about 50 or about 60 months post surgical intervention to remove breast cancer tumor(s). A “poor” outcome may also be about a 70% or less survival rate after about 40 months, or about a 80% or less survival rate after about 20 months, post surgical intervention.

In one embodiment of the invention, the isolation and analysis of a breast cancer cell sample may be performed as follows:
(1) Ductal lavage or other non-invasive procedure is performed on a patient to obtain a sample.
(2) Sample is prepared and coated onto a microscope slide. Note that ductal lavage results in clusters of cells that are cytologically examined as stated above.
(3) Pathologist or image analysis software scans the sample for the presence of non-normal and/or atypical cells.
(4) If non-normal and/or atypical cells are observed, those cells are harvested (e.g. by microdissection such as LCM).
(5) RNA is extracted from the harvested cells.
(6) RNA is purified, amplified, and labeled.
(7) Labeled nucleic acid is contacted with a microarray containing polynucleotides of the genes identified herein as correlated to discriminations between breast cancer survival outcomes under hybridization conditions, then processed and scanned to obtain a pattern of intensities of each spot (relative to a control for general gene expression in cells) which determine the level of expression of the gene(s) in the cells.
(8) The pattern of intensities is analyzed by comparison to the expression patterns of the genes in known samples of breast cancer cells correlated with survival outcomes (relative to the same control).

A specific example of the above method would be performing ductal lavage following a primary screen, observing and collecting non-normal and/or atypical cells for analysis. The comparison to known expression patterns, such as that made possible by a model generated by an algorithm (such as, but not limited to nearest neighbor type analysis, SVM, or neural networks) with reference gene expression data for the different breast cancer survival outcomes, identifies the cells as being correlated with subjects with good outcomes. Another example would be taking a breast tumor removed from a subject after surgical intervention, isolation and preparation of breast cancer cells for determination/identification of atypical, non-normal, or cancer cells, and isolation of said cells followed by steps 5 through 8 above.

Alternatively, the sample may permit the collection of both normal as well as cancer cells for analysis. The gene expression patterns for each of these two samples will be compared to each other as well as the model and the normal versus individual comparisons therein based upon the reference data set. This approach can be significantly more powerful that the cancer cells only approach because it utilizes significantly more information from the normal cells and the differences between normal and non-normal or atypical or cancer cells (in both the sample and reference data sets) to determine the likely survival outcome of the patient based on gene expression in the cancer cells from the sample.

With use of the present invention, skilled physicians may prescribe treatments based on prognosis determined via non-invasive samples that they would have prescribed for a patient which had previously received a diagnosis via a solid tissue biopsy.

The above discussion is also applicable where a palpable lesion is detected followed by fine needle aspiration or needle biopsy of cells from the breast. The cells are plated and reviewed by a pathologist or automated imaging system which selects cells for analysis as described above.

The present invention may also be used, however, with solid tissue biopsies. For example, a solid biopsy may be collected and prepared for visualization followed by determination of expression of one or more genes identified herein to determine the breast cancer survival outcome. One preferred means is by use of in situ hybridization with polynucleotide or protein identifying probe(s) for assaying expression of said gene(s).

In an alternative method, the solid tissue biopsy may be used to extract molecules followed by analysis for expression of one or more gene(s). This provides the possibility of leaving out the need for visualization and collection of only cancer cells or cells suspected of being cancerous. This method may of course be modified such that only cells that have been positively selected are collected and used to extract molecules for analysis. This would require visualization and selection as an prerequisite to gene expression analysis.

In a further modification of the above, both normal cells and cancer cells are collected and used to extract molecules for analysis of gene expression. The approach, benefits and results are as described above using non-invasive sampling.

The genes identified herein may be used to generate a model capable of predicting the breast cancer survival outcomes via an unknown breast cell sample based on the expression of the identified genes in the sample. Such a model may be generated by any of the algorithms described herein or otherwise known in the art as well as those recognized as equivalent in the art using gene(s) (and subsets thereof) disclosed herein for the identification of breast cancer outcomes. The model provides a means for comparing expression profiles of gene(s) of the subset from the sample against the profiles of reference data used to build the model. The model can compare the sample profile against each of the reference profiles or against model defining delineations made based upon the reference profiles. Additionally, relative values from the sample profile may be used in comparison with the model or reference profiles.

In a preferred embodiment of the invention, breast cell samples identified as normal and cancerous from the same subject may be analyzed for their expression profiles of the genes used to generate the model. This provides an advantageous means of identifying survival outcomes based on relative differences from the expression profile of the normal sample. These differences can then be used in comparison to differences between normal and individual cancerous reference data which was also used to generate the model.

The detection of gene expression from the samples may be by use of a single microarray able to assay gene expression from some or all genes disclosed herein for convenience and accuracy.

Other uses of the present invention include providing the ability to identify breast cancer cell samples as correlated with particular breast cancer survival outcomes for further research or study. This provides a particular advantage in many contexts requiring the identification of cells based on objective genetic or molecular criteria.

The materials for use in the methods of the present invention are ideally suited for preparation of kits produced in accordance with well known procedures. The invention thus provides kits comprising agents for the detection of expression of the disclosed genes for identifying breast cancer survival outcomes. Such kits optionally comprising the agent with an identifying description or label or instructions relating to their use in the methods of the present invention, is provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

The methods provided by the present invention may also be automated in whole or in part. All aspects of the present invention may also be practiced such that they consist essentially of a subset of the disclosed genes to the exclusion of material irrelevant to the identification of breast cancer survival outcomes via a cell containing sample.

Having now generally described the invention, the same will be more readily understood through reference to the following examples which are provided by way of illustration, and are not intended to be limiting of the present invention, unless specified.

### EXAMPLES

### Example I

### Materials and Methods

Clinical specimen collection and clinicopathological parameters. 86 patients were expression profiled, 57 of these had clinical follow-up, specifically overall survival. Biomarker status is shown below in Table 1 for all 86 patients

TABLE 1
Age and biomarker status for the 86 patients
subsequently gene expression profiled

| Age | | No. of Cases | Percentage |
|---|---|---|---|
| | <45 | 12 | 14% |
| | 45-55 | 24 | 28% |
| | >55 | 50 | 58% |
| Estrogen-receptor status | | | |
| | positive | 41 | 48% |
| | negative | 45 | 52% |
| Progesterone-receptor status | | | |
| | positive | 32 | 37% |
| | negative | 54 | 63% |
| Her2/Neu status | | | |
| | positive | 16 | 19% |
| | intermediate | 23 | 27% |
| | negative | 45 | 54% |

### Example II

### Identification of ER positive subtypes with different survival outcomes

Within the set of 86 patients from Example I, 41 had breast tumors that were ER+ via a biomarker test. Within this set of 41, microdissection was used to obtain breast cancer cells for identification of a molecular signature (i.e., expression of genes) that differentially categorized the ER+ group into two subgroups. This was done by (i) using unsupervised hierarchical clustering to identify two subtypes, followed by (ii) completing a t-test on every gene and (iii) extracting those genes whose differential expression was at an adjusted p <0.05 (using false discovery rate procedure).

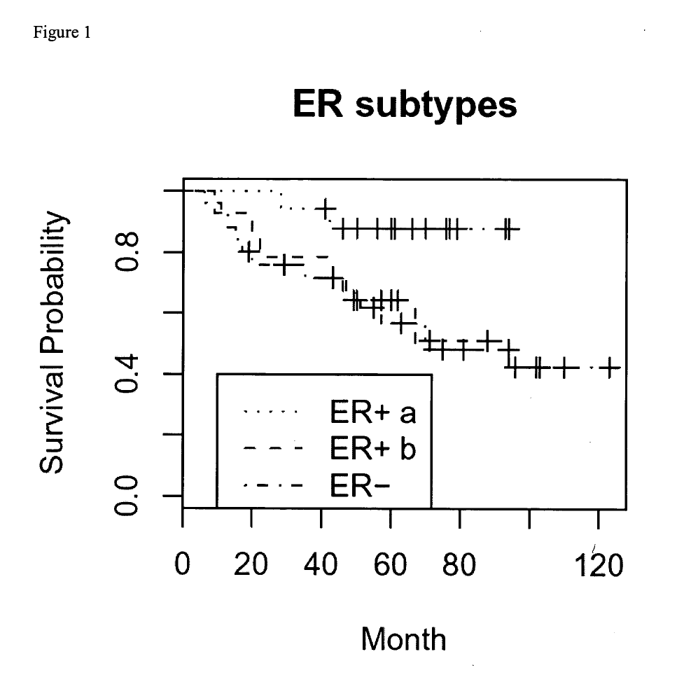

864 genes were extracted and are listed in Tables 2 and 3. Using clinical outcome (overall survival), it was determined that these two subtypes (identified as ERa and ERb, or ER positive subtypes a and b) divided the ER+ patients into two different survival curves as shown in FIG. 1. Genes which which positively correlate with (are overexpressed in) the ERa subtype are negatively correlated with (are underexpressed in) the ERb subtype. Conversely, genes which which positively correlate with (are overexpressed in) the ERb subtype are negatively correlated with (are underexpressed in) the ERa subtype.

It is interesting to note that the ERb subtype has a similar survival as those patients whose tumors were ER negative. As such, one aspect of the invention includes the treatment of patients with breast cancer cells having the ERb subtype in the manner of treating patients with cells having an ER negative phenotype.

TABLE 2
Genes, the expressions of which positively correlate with the ERa subtype

| Clone_ID | P_value | Gene_Description |
|---|---|---|
| 504187 | 3.31E−02 | ESTs, Moderately similar to ALU8_HUMAN ALU SUBFAMILY SX SEQUENCE CONTAMINATION WARNING ENTRY |
| | | [H. sapiens] |
| 71763 | 1.78E−02 | SIP\|Siah-interacting protein |
| 2048524 | 1.67E−02 | JAK2\|Janus kinase 2 (a protein tyrosine kinase) |
| 898242 | 1.12E−02 | SRPR\|signal recognition particle receptor (docking protein) |
| 1709791 | 7.86E−03 | BAIAP1\|BAI1-associated protein 1 |
| 110578 | 4.22E−02 | ESTs |
| 50713 | 4.35E−02 | KIAA1577\|KIAA1577 protein |
| 594517 | 2.44E−02 | SFRS6\|splicing factor, arginine/serine-rich 6 |
| 41826 | 2.67E−03 | Homo sapiens cDNA FLJ32064 fis, clone OCBBF1000080 |
| 814620 | 1.83E−02 | FBP17\|formin-binding protein 17 |
| 1160558 | 4.31E−03 | B-DIOX-II\|putative b,b-carotene-9,10-dioxygenase |
| 809879 | 4.21E−02 | FLJ10307\|hypothetical protein FLJ10307 |
| 298134 | 2.68E−02 | FZD1\|frizzled (Drosophila) homolog 1 |
| 325515 | 3.20E−03 | FLJ10980\|hypothetical protein FLJ10980 |
| 782306 | 1.30E−02 | FLJ13110\|hypothetical protein FLJ13110 |
| 48518 | 2.11E−02 | Homo sapiens mRNA for KIAA1888 protein, partial cds |
| 1636035 | 2.82E−03 | GASC1\|gene amplified in squamous cell carcinoma 1 |
| 129644 | 8.78E−03 | SSH3BP1\|spectrin SH3 domain binding protein 1 |
| 1866068 | 4.96E−02 | ESTs |
| 1685642 | 3.86E−02 | PMP2\|peripheral myelin protein 2 |
| 366966 | 3.22E−02 | Homo sapiens cDNA: FLJ21333 fis, clone COL02535 |
| 281904 | 1.22E−02 | KIAA0349\|KIAA0349 protein |
| 1926007 | 3.81E−02 | EST |
| 825053 | 1.41E−03 | Homo sapiens mRNA; cDNA DKFZp434J0828 (from clone DKFZp434J0828) |
| 346643 | 3.93E−02 | ESTs |
| 1683035 | 3.40E−02 | ESTs |
| 795342 | 3.17E−02 | ESTs |
| 130116 | 8.42E−03 | ESTs |
| 347378 | 4.90E−02 | FLJ12492\|hypothetical protein FLJ12492 |
| 491545 | 4.86E−02 | KIAA0965\|KIAA0965 protein |
| 812964 | 3.36E−03 | Homo sapiens mRNA full length insert cDNA clone EUROIMAGE 2068962 |
| 2139152 | 2.06E−02 | PDZ-GEF1\|PDZ domain containing guanine nucleotide exchange factor(GEF)1 |
| 502818 | 1.30E−02 | ARHA\|ras homolog gene family, member A |
| 1636111 | 1.30E−02 | HNRPU\|heterogeneous nuclear ribonucleoprotein U (scaffold attachment factor A) |
| 1492780 | 4.52E−02 | ESTs, Weakly similar to I38022 hypothetical protein [H. sapiens] |
| 813697 | 1.02E−02 | KIAA0746\|KIAA0746 protein |
| 810282 | 8.98E−03 | ITPK1\|inositol 1,3,4-triphosphate 5/6 kinase |
| 845454 | 4.44E−02 | Homo sapiens cDNA: FLJ23597 fis, clone LNG15281 |
| 154657 | 3.22E−02 | Homo sapiens cDNA: FLJ21286 fis, clone COL01915 |
| 293063 | 1.33E−02 | POLR2B\|polymerase (RNA) II (DNA directed) polypeptide B (140 kD) |
| 753973 | 1.98E−02 | NFAT5\|nuclear factor of activated T-cells 5, tonicity-responsive |
| 969495 | 1.30E−02 | TIGA1\|TIGA1 |
| 786605 | 2.18E−02 | APG-1\|heat shock protein (hsp110 family) |
| 417884 | 4.91E−03 | Homo sapiens cDNA FLJ12052 fis, clone HEMBB1002042, moderately similar to CYTOCHROME P450 4C1 (EC |
| | | 1.14.14.1) |
| 325606 | 1.97E−02 | EST |
| 201282 | 7.98E−03 | DKFZP434N126\|DKFZP434N126 protein |
| 773502 | 6.44E−03 | ESTs, Weakly similar to S65824 reverse transcriptase homolog [H. sapiens] |
| 812975 | 1.42E−02 | KIAA0172\|KIAA0172 protein |
| 162753 | 1.29E−02 | DD5\|progestin induced protein |
| 712460 | 1.49E−03 | NKTR\|natural killer-tumor recognition sequence |
| 359836 | 1.51E−03 | FLJ10726\|hypothetical protein FLJ10726 |
| 845609 | 3.48E−02 | LOC90701\|similar to signal peptidase complex (18 kD) |
| 251698 | 1.02E−02 | FBXW1B\|f-box and WD-40 domain protein 1B |
| 136954 | 3.58E−02 | ESTs, Weakly similar to YEX0_YEAST HYPOTHETICAL 64.8 KDA PROTEIN IN GDI1-COX15 INTERGENIC REGION |
| | | [S. cerevisiae] |
| 283453 | 3.98E−02 | Homo sapiens cDNA FLJ11458 fis, clone HEMBA1001557 |
| 267419 | 3.17E−02 | ESTs |
| 140837 | 4.30E−02 | CLPX\|ClpX (caseinolytic protease X, E. coli) homolog |
| 753987 | 4.05E−02 | ADPRTL1\|ADP-ribosyltransferase (NAD+; poly (ADP-ribose) polymerase)-like 1 |
| 825076 | 4.96E−03 | APT6M8-9\|ATPase, H+ transporting, lysosomal (vacuolar proton pump) membrane sector associated protein M8-9 |
| 813854 | 1.28E−03 | PURA\|purine-rich element binding protein A |
| 812042 | 4.03E−02 | TSC1\|tuberous sclerosis 1 |
| 491565 | 3.64E−02 | CITED2\|Cbp/p300-interacting transactivator, with Glu/Asp-rich carboxy-terminal domain, 2 |
| 782331 | 4.50E−04 | ESTs |
| 415288 | 2.73E−02 | SRP46\|Splicing factor, arginine/serine-rich, 46 kD |
| 149058 | 2.27E−02 | Homo sapiens cDNA FLJ10174 fis, clone HEMBA1003959 |
| 287745 | 3.16E−02 | Homo sapiens cDNA FLJ30482 fis, clone BRAWH2000034, moderately similar to TRP-185 protein |
| 897625 | 2.69E−02 | KIAA0532\|KIAA0532 protein |
| 757337 | 3.93E−02 | ESTs |
| 773375 | 3.39E−02 | EST |
| 284261 | 2.17E−02 | MDS030\|uncharacterized hematopoietic stem/progenitor cells protein MDS030 |
| 843008 | 4.67E−02 | GC20\|translation factor sui1 homolog |
| 1461120 | 4.06E−02 | DLEU2\|deleted in lymphocytic leukemia, 2 |
| 1933255 | 3.24E−02 | DNAJA4\|DnaJ (Hsp40) homolog, subfamily A, member 4 |
| 50685 | 4.17E−02 | KIAA1414\|KIAA1414 protein |
| 824354 | 2.44E−02 | GRLF1\|glucocorticoid receptor DNA binding factor 1 |
| 259267 | 3.39E−02 | Homo sapiens mRNA; cDNA DKFZp586N2424 (from clone DKFZp586N2424) |
| 361048 | 3.59E−02 | p100\|EBNA-2 co-activator (100 kD) |
| 279800 | 3.82E−02 | SLMAP\|sarcolemma associated protein |
| 1603583 | 4.70E−02 | SH3BGRL\|SH3 domain binding glutamic acid-rich protein like |
| 1558561 | 2.71E−02 | ATRN\|attractin |
| 135303 | 2.91E−04 | HT007\|uncharacterized hypothalamus protein HT007 |
| 287683 | 1.12E−02 | KIAA1387\|KIAA1387 protein |
| 844680 | 8.98E−03 | TRD@\|T cell receptor delta locus |
| 279665 | 2.65E−02 | PDX1\|Pyruvate dehydrogenase complex, lipoyl-containing component X; E3-binding protein |
| 53092 | 1.82E−02 | KIAA0436\|putative L-type neutral amino acid transporter |
| 376697 | 8.98E−03 | Homo sapiens cDNA FLJ30060 fis, clone ADRGL2000097 |
| 126413 | 4.52E−02 | ITIH2\|inter-alpha (globulin) inhibitor, H2 polypeptide |
| 268234 | 3.74E−02 | DMXL1\|Dmx-like 1 |
| 363590 | 3.47E−02 | ARNT2\|aryl hydrocarbon receptor nuclear translocator 2 |
| 814673 | 2.44E−02 | DKFZP547E2110\|DKFZP547E2110 protein |
| 268240 | 3.67E−02 | FXC1\|fracture callus 1 (rat) homolog |
| 346902 | 2.06E−03 | Homo sapiens cDNA: FLJ21985 fis, clone HEP06226 |
| 46896 | 3.10E−02 | PRO1331\|hypothetical protein PRO1331 |
| 825240 | 4.61E−02 | ESTs, Weakly similar to SFRB_HUMAN SPLICING FACTOR ARGININE/SERINE-RICH 11 [H. sapiens] |
| 42827 | 4.97E−02 | Homo sapiens cDNA FLJ31604 fis, clone NT2RI2002699 |
| 138589 | 2.24E−04 | Homo sapiens clone 24538 mRNA sequence |
| 797062 | 1.14E−02 | ESTs |
| 1587863 | 2.88E−02 | ACAA1\|acetyl-Coenzyme A acyltransferase 1 (peroxisomal 3-oxoacyl-Coenzyme A thiolase) |
| 841287 | 2.44E−02 | GNPAT\|glyceronephosphate O-acyltransferase |
| 742581 | 1.67E−02 | Homo sapiens cDNA FLJ10366 fis, clone NT2RM2001420 |
| 823574 | 4.90E−02 | Homo sapiens cDNA FLJ33111 fis, clone TRACH2001085 |
| 343352 | 3.45E−02 | KIAA1134\|KIAA1134 protein |
| 2013633 | 1.33E−02 | STAM\|signal transducing adaptor molecule (SH3 domain and ITAM motif) 1 |
| 261492 | 2.69E−02 | LCHN\|LCHN protein |
| 712641 | 2.35E−02 | TPR\|translocated promoter region (to activated MET oncogene) |
| 199637 | 3.82E−02 | Homo sapiens cDNA FLJ31102 fis, clone IMR322000010 |
| 624291 | 4.07E−02 | GHITM\|growth hormone inducible transmembrane protein |
| 134525 | 3.82E−02 | CUL3\|cullin 3 |
| 141815 | 3.93E−02 | JAG1\|jagged 1 (Alagille syndrome) |
| 161998 | 3.97E−02 | FLJ23138\|hypothetical protein FLJ23138 |
| 345032 | 3.67E−02 | ESTs |
| 1712148 | 3.86E−02 | RNU17D\|RNA, U17D small nucleolar |
| 280154 | 1.77E−02 | SYNJ2\|synaptojanin 2 |
| 814906 | 2.91E−02 | KIAA0648\|KIAA0648 protein |
| 768940 | 2.28E−02 | KIAA0874\|KIAA0874 protein |
| 812153 | 1.60E−03 | FLJ13081\|hypothetical protein FLJ13081 |
| 490945 | 4.45E−04 | ESTs |
| 812155 | 2.18E−02 | RABGGTB\|Rab geranylgeranyltransferase, beta subunit |
| 741795 | 3.22E−02 | RALGPS1A\|Ral guanine nucleotide exchange factor RalGPS1A |
| 768008 | 2.11E−02 | BAG2\|BCL2-associated athanogene 2 |
| 758318 | 2.55E−02 | FBXO3\|F-box only protein 3 |
| 753300 | 1.66E−03 | DKFZp586F1019\|DKFZp586F1019 protein |
| 839094 | 1.18E−02 | CRYBA1\|crystallin, beta A1 |
| 754033 | 2.07E−02 | LZTFL1\|leucine zipper transcription factor-like 1 |
| 897595 | 1.16E−02 | CBFA2T2\|core-binding factor, runt domain, alpha subunit 2; translocated to, 2 |
| 726703 | 3.48E−02 | Homo sapiens clone 23736 mRNA sequence |
| 1631238 | 2.28E−02 | KIAA1483\|KIAA1483 protein |
| 812300 | 1.36E−02 | FLJ20265\|hypothetical protein FLJ20265 |
| 788264 | 2.82E−02 | DPAGT1\|dolichyl-phosphate (UDP-N-acetylglucosamine) N-acetylglucosaminephosphotransferase 1 (GlcNAc-1-P |
| | | transferase) |
| 84229 | 2.97E−02 | GK003\|GK003 protein |
| 120561 | 4.28E−02 | KIDINS220\|likely homolog of rat kinase D-interacting substance of 220 kDa |
| 786592 | 2.72E−02 | ZNF265\|zinc finger protein 265 |
| 1884135 | 2.82E−02 | ESTs |
| 731318 | 2.82E−02 | KIAA0981\|KIAA0981 protein |
| 700500 | 4.96E−03 | PCTK2\|PCTAIRE protein kinase 2 |
| 358151 | 2.73E−02 | ZNF33A\|zinc finger protein 33a (KOX 31) |
| 897670 | 1.90E−02 | Human transposon-like element mRNA |
| 754040 | 2.02E−02 | Homo sapiens cDNA FLJ31626 fis, clone NT2RI2003317 |
| 53276 | 2.06E−03 | Homo sapiens clone 24538 mRNA sequence |
| 454459 | 1.93E−02 | Homo sapiens clone 23870 mRNA sequence |
| 1535953 | 1.44E−02 | ESTs |
| 266747 | 1.07E−02 | Homo sapiens, Similar to RIKEN cDNA 2010001O09 gene, clone MGC: 21387 IMAGE: 4471592, mRNA, complete cds |
| 1584623 | 7.56E−03 | CCNC\|cyclin C |
| 726571 | 8.61E−03 | SMBP\|SM-11044 binding protein |
| 1582956 | 8.15E−03 | DKFZP434O1427\|hypothetical protein DKFZp434O1427 |
| 757462 | 1.50E−02 | E2IG5\|hypothetical protein, estradiol-induced |
| 1707637 | 3.71E−02 | ESTs |
| 815800 | 4.87E−03 | FLJ21343\|hypothetical protein FLJ21343 |
| 825350 | 2.91E−04 | KIAA1040\|KIAA1040 protein |
| 840664 | 1.72E−02 | EST |
| 50887 | 7.87E−03 | RALGDS\|ral guanine nucleotide dissociation stimulator |
| 503914 | 4.28E−02 | KIAA1311\|KIAA1311 protein |
| 884657 | 4.35E−02 | TIMM8B\|translocase of inner mitochondrial membrane 8 (yeast) homolog B |
| 469172 | 9.21E−03 | SEC22C\|vesicle trafficking protein |
| 685516 | 2.70E−02 | GPCR150\|putative G protein-coupled receptor |
| 767091 | 3.45E−02 | Homo sapiens PAC clone RP1-130H16 from 22q12.1-qter |
| 323074 | 2.67E−03 | HMG2L1\|high-mobility group protein 2-like 1 |
| 1636349 | 1.69E−02 | 15-Sep\|15 kDa selenoprotein |
| 753404 | 3.35E−02 | KIAA0887\|KIAA0887 protein |
| 291908 | 1.77E−02 | CTNND1\|catenin (cadherin-associated protein), delta 1 |
| 1694775 | 2.60E−02 | EST |
| 1030349 | 4.78E−02 | DFFB\|DNA fragmentation factor, 40 kD, beta polypeptide (caspase-activated DNase) |
| 34852 | 2.19E−02 | BIRC2\|baculoviral IAP repeat-containing 2 |
| 277185 | 2.88E−02 | PRO0461\|PRO0461 protein |
| 210610 | 3.88E−03 | CEP1\|centrosomal protein 1 |
| 277187 | 1.66E−02 | MKP-7\|MAPK phosphatase-7 |
| 825363 | 4.70E−02 | ESTs |
| 49562 | 8.44E−03 | KIAA0171\|KIAA0171 gene product |
| 767170 | 3.88E−02 | LOC51606\|CGI-11 protein |
| 784085 | 4.31E−03 | TUSP\|tubby super-family protein |
| 1650934 | 1.78E−02 | Homo sapiens cDNA FLJ11472 fis, clone HEMBA1001711 |
| 1030351 | 3.48E−03 | SCYB11\|small inducible cytokine subfamily B (Cys-X-Cys), member 11 |
| 701402 | 1.50E−03 | Crk\|v-crk avian sarcoma virus CT10 oncogene homolog |
| 2062429 | 3.41E−02 | PRO2730\|hypothetical protein PRO2730 |
| 28444 | 4.45E−04 | CRSP2\|cofactor required for Sp1 transcriptional activation, subunit 2 (150 kD) |
| 197077 | 2.86E−02 | GOLPH3\|golgi phosphoprotein 3 (coat-protein) |
| 826245 | 2.95E−02 | LOC54505\|hypothetical protein |
| 1586251 | 1.80E−02 | LOC51030\|CGI-148 protein |
| 841485 | 1.17E−02 | Homo sapiens cDNA FLJ31058 fis, clone HSYRA2000828 |
| 752547 | 3.01E−04 | Homo sapiens mRNA; cDNA DKFZp586G1520 (from clone DKFZp586G1520) |
| 511012 | 4.21E−02 | AGPS\|alkylglycerone phosphate synthase |
| 68225 | 2.68E−02 | Homo sapiens pTM5 mariner-like transposon mRNA, partial sequence |
| 121470 | 3.30E−02 | BCCIP\|BRCA2 and CDKN1A-interacting protein |
| 360539 | 4.39E−02 | PPP3CB\|protein phosphatase 3 (formerly 2B), catalytic subunit, beta isoform (calcineurin A beta) |
| 782700 | 4.89E−02 | CLASP2\|CLIP-associating protein 2 |
| 80050 | 4.43E−02 | FLJ23153\|likely ortholog of mouse tumor necrosis-alpha-induced adipose-related protein |
| 343555 | 1.97E−02 | Homo sapiens mRNA; cDNA DKFZp586D0923 (from clone DKFZp586D0923) |
| 108425-2 | 4.52E−02 | ESTs, Weakly similar to JC5314 CDC28/cdc2-like kinase associating arginine-serine cyclophilin [H. sapiens] |
| 289716 | 2.24E−04 | Homo sapiens mRNA; cDNA DKFZp566P1124 (from clone DKFZp566P1124) |
| 1688510 | 2.81E−02 | Homo sapiens CLK4 mRNA, complete cds |
| 1636360 | 8.61E−03 | FLJ14957\|hypothetical protein FLJ14957 |
| 713647 | 1.77E−02 | TSPAN-3\|tetraspan 3 |
| 136324 | 3.35E−02 | Homo sapiens PAK2 mRNA, complete cds |
| 51851 | 4.88E−03 | ESTs, Weakly similar to I78885 serine/threonine-specific protein kinase [H. sapiens] |
| 897926 | 2.51E−03 | Homo sapiens clone FLB5227 PRO1367 mRNA, complete cds |
| 588368 | 2.68E−02 | KIAA0947\|KIAA0947 protein |
| 29185 | 2.82E−02 | ULK2\|unc-51 (C. elegans)-like kinase 2 |
| 825451 | 3.08E−02 | P115\|vesicle docking protein p115 |
| 195557 | 3.08E−02 | FLJ10842\|hypothetical protein FLJ10842 |
| 1499864 | 4.31E−02 | ESTs |
| 254625 | 2.11E−02 | KIAA0229\|KIAA0229 protein |
| 1435481 | 2.07E−02 | Homo sapiens mRNA; cDNA DKFZp586G2222 (from clone DKFZp586G2222) |
| 1911706 | 3.17E−02 | GA\|breast cell glutaminase |
| 795677 | 3.36E−02 | Homo sapiens cDNA: FLJ21314 fis, clone COL02248 |
| 343566 | 1.97E−02 | FLJ23342\|hypothetical protein FLJ23342 |
| 564847 | 9.88E−03 | Homo sapiens cDNA FLJ30861 fis, clone FEBRA2003541 |
| 322511 | 3.35E−02 | Homo sapiens mRNA; cDNA DKFZp564D1462 (from clone DKFZp564D1462) |
| 1556322 | 3.36E−03 | EST |
| 768064 | 2.23E−02 | CYP1A1\|cytochrome P450, subfamily\|(aromatic compound-inducible), polypeptide 1 |
| 358344 | 1.04E−02 | KIAA0244\|KIAA0244 protein |
| 1556259 | 4.47E−02 | ALAD\|aminolevulinate, delta-, dehydratase |
| 753430 | 1.14E−02 | ATRX\|alpha thalassemia/mental retardation syndrome X-linked (RAD54 (S. cerevisiae) homolog) |
| 669367 | 4.28E−02 | USP15\|ubiquitin specific protease 15 |
| 809421 | 1.76E−02 | PCBD\|6-pyruvoyl-tetrahydropterin synthase/dimerization cofactor of hepatocyte nuclear factor 1 alpha (TCF1) |
| 704697 | 4.98E−02 | HERC3\|hect domain and RLD 3 |
| 1551317 | 1.91E−02 | EST |
| 772888 | 4.03E−02 | KIAA1012\|KIAA1012 protein |
| 825394 | 2.28E−02 | DJ465N24.2.1\|hypothetical protein dJ465N24.2.1 |
| 73933 | 4.09E−02 | ESTs |
| 261852 | 1.77E−02 | ESTs |
| 241530 | 1.28E−03 | EPHA2\|EphA2 |
| 1635650 | 1.02E−02 | KIAA0576\|KIAA0576 protein |
| 772962 | 2.36E−02 | Homo sapiens cDNA FLJ31149 fis, clone IMR322001491, moderately similar to Rattus norvegicus tricarboxylate |
| | | carrier-like protein mRNA |
| 782587 | 6.22E−03 | UBE4A\|ubiquitination factor E4A (homologous to yeast UFD2) |
| 825615 | 1.34E−02 | ESTs |
| 823871 | 3.34E−02 | SPARCL1\|SPARC-like 1 (mast9, hevin) |
| 769022 | 2.44E−02 | GNAQ\|guanine nucleotide binding protein (G protein), q polypeptide |
| 1584755 | 1.22E−02 | ESTs |
| 814983 | 7.10E−03 | FLJ11068\|hypothetical protein FLJ11068 |
| 810843 | 4.22E−02 | BM029\|uncharacterized bone marrow protein BM029 |
| 70606 | 1.97E−02 | ESTs |
| 322537 | 1.67E−02 | Homo sapiens cDNA: FLJ21425 fis, clone COL04162 |
| 289677 | 3.55E−02 | CG005\|hypothetical protein from BCRA2 region |
| 701371 | 1.27E−03 | Homo sapiens mRNA; cDNA DKFZp586I1518 (from clone DKFZp586I1518) |
| 745360 | 2.35E−02 | HAT1\|histone acetyltransferase 1 |
| 754255 | 3.25E−02 | ESTs |
| 85313 | 2.99E−02 | KIAA1254\|KIAA1254 protein |
| 141972 | 4.44E−02 | ITM1\|integral membrane protein 1 |
| 745437 | 2.37E−02 | ESTs |
| 280456 | 2.99E−02 | EST |
| 788555 | 1.27E−03 | DKFZP564I052\|DKFZP564I052 protein |
| 202577 | 4.55E−02 | HNMT\|histamine N-methyltransferase |
| 813187 | 8.91E−03 | Homo sapiens cDNA: FLJ21264 fis, clone COL01579 |
| 502096 | 9.88E−03 | Homo sapiens mRNA; cDNA DKFZp761K2024 (from clone DKFZp761K2024) |
| 753602 | 3.68E−02 | FLJ10618\|hypothetical protein FLJ10618 |
| 487301 | 2.69E−02 | FBXL5\|f-box and leucine-rich repeat protein 5 |
| 488033 | 1.42E−02 | DNAJB9\|DnaJ (Hsp40) homolog, subfamily B, member 9 |
| 364865 | 2.77E−03 | FLJ21062\|hypothetical protein FLJ21062 |
| 267691 | 2.58E−04 | FLJ20360\|hypothetical protein FLJ20360 |
| 788705 | 6.25E−03 | USF1\|upstream transcription factor 1 |
| 124138 | 1.45E−02 | NXF1\|nuclear RNA export factor 1 |
| 813261 | 1.40E−02 | Homo sapiens clone 23645 mRNA sequence |
| 856454 | 3.01E−04 | SLC3A2\|solute carrier family 3 (activators of dibasic and neutral amino acid transport), member 2 |
| 470861 | 4.57E−02 | NDUFB6\|NADH dehydrogenase (ubiquinone) 1 beta subcomplex, 6 (17 kD, B17) |
| 143661 | 3.41E−02 | NTN4\|netrin 4 |
| 665405 | 2.18E−02 | MYO5C\|myosin 5C |
| 303109 | 1.27E−03 | P2Y5\|purinergic receptor (family A group 5) |
| 1470365 | 3.98E−02 | ST7\|suppression of tumorigenicity 7 |
| 220372 | 4.61E−02 | HS3ST1\|heparan sulfate (glucosamine) 3-O-sulfotransferase 1 |
| 814214 | 7.66E−03 | D8S2298E\|reproduction 8 |
| 796739 | 4.09E−02 | MGC10924\|hypothetical protein MGC10924 similar to Nedd4 WW-binding protein 5 |
| 786109 | 9.38E−04 | ESTs |
| 1637504 | 1.66E−03 | EST |
| 48033 | 1.86E−02 | ESTs |
| 1557318 | 4.43E−02 | ESTs |
| 2292807 | 3.15E−03 | ACAT1\|acetyl-Coenzyme A acetyltransferase 1 (acetoacetyl Coenzyme A thiolase) |
| 1034776 | 9.51E−03 | AD037\|AD037 protein |
| 295255 | 1.78E−02 | KIAA0254\|KIAA0254 gene product |
| 306380 | 2.37E−03 | MGC4276\|hypothetical protein MGC4276 similar to CG8198 |
| 1641245 | 2.06E−03 | LOC51320\|hypothetical protein |
| 303043 | 2.19E−02 | ESTs, Weakly similar to G02075 transcription repressor zinc finger protein 85 [H. sapiens] |
| 752752 | 7.56E−03 | ESTs |
| 358468 | 1.95E−02 | RNF11\|ring finger protein 11 |
| 363146 | 3.46E−02 | PPP3R1\|protein phosphatase 3 (formerly 2B), regulatory subunit B (19 kD), alpha isoform (calcineurin B, type I) |
| 84613 | 1.67E−02 | DKFZP564K247\|DKFZP564K247 protein |
| 1519143 | 2.28E−02 | RISC\|likely homolog of rat and mouse retinoid-inducible serine carboxypeptidase |
| 825582 | 4.62E−02 | Homo sapiens mRNA; cDNA DKFZp564O0122 (from clone DKFZp564O0122) |
| 789383 | 1.97E−02 | CREM\|cAMP responsive element modulator |
| 813424 | 1.41E−02 | PPID\|peptidylprolyl isomerase D (cyclophilin D) |
| 22917 | 1.89E−02 | Homo sapiens mRNA; cDNA DKFZp761M0111 (from clone DKFZp761M0111) |
| 1593829 | 3.51E−02 | TIA1\|TIA1 cytotoxic granule-associated RNA-binding protein |
| 1578447 | 2.28E−02 | Homo sapiens cDNA FLJ31866 fis, clone NT2RP7001745 |
| 362279 | 2.60E−02 | ARHGEF5\|Rho guanine nucleotide exchange factor (GEF) 5 |
| 1540949 | 3.24E−02 | EST |
| 155118 | 1.78E−02 | ESTs |
| 321770 | 1.15E−02 | FBP17\|formin-binding protein 17 |
| 854874 | 1.30E−02 | KIAA0212\|KIAA0212 gene product |
| 43977 | 4.70E−03 | KIAA0182\|KIAA0182 protein |
| 136399 | 8.91E−03 | DKFZP586F2423\|hypothetical protein DKFZp586F2423 |
| 229901 | 1.97E−02 | CTSO\|cathepsin O |
| 726890 | 4.87E−02 | MGC4643\|hypothetical protein MGC4643 |
| 743876 | 1.97E−02 | MBLR\|MBLR protein |
| 809488 | 2.82E−02 | RAI17\|retinoic acid induced 17 |
| 1572710 | 2.34E−02 | FLJ21213\|hypothetical protein FLJ21213 |
| 155050 | 2.58E−04 | MDS025\|hypothetical protein MDS025 |
| 782851 | 1.70E−02 | FLJ12799\|hypothetical protein FLJ12799 |
| 2011515 | 1.98E−02 | DKFZP586B0923\|DKFZP586B0923 protein |
| 1602284 | 2.60E−02 | EST |
| 781046 | 4.95E−02 | ERBB2IP\|erbb2-interacting protein ERBIN |
| 767477 | 2.03E−02 | ANKRA2\|ankyrin repeat, family A (RFXANK-like), 2 |
| 179804 | 2.57E−02 | PWP2H\|PWP2 (periodic tryptophan protein, yeast) homolog |
| 365919 | 3.42E−02 | STAU\|staufen (Drosophila, RNA-binding protein) |
| 50339 | 1.32E−02 | ESTs, Moderately similar to hypothetical protein [H. sapiens] |
| 1598787 | 1.32E−02 | FLJ20730\|hypothetical protein FLJ20730 |
| 2103000 | 1.74E−02 | ESTs |
| 840984 | 2.53E−02 | CAV2\|caveolin 2 |
| 788745 | 1.77E−02 | WS-3\|novel RGD-containing protein |
| 1558212 | 1.58E−03 | ESTs |
| 813518 | 3.88E−02 | ESTs |
| 143661-2 | 1.36E−02 | NTN4\|netrin 4 |
| 811918 | 4.54E−02 | KIAA0952\|KIAA0952 protein |
| 951125 | 3.36E−02 | PECI\|peroxisomal D3,D2-enoyl-CoA isomerase |
| 811849 | 1.30E−02 | MGC5521\|hypothetical protein MGC5521 |
| 298769 | 4.52E−02 | KEO4\|similar to Caenorhabditis elegans protein C42C1.9 |
| 897142 | 1.36E−02 | MAP2K1IP1\|mitogen-activated protein kinase kinase 1 interacting protein 1 |
| 754450 | 3.27E−02 | ARHGEF12\|Rho guanine exchange factor (GEF) 12 |
| 214131 | 4.61E−02 | NIT2\|Nit protein 2 |
| 143846 | 4.77E−02 | LRP2\|low density lipoprotein-related protein 2 |
| 2028916 | 3.84E−02 | Homo sapiens mRNA for Hmob33 protein, 3 untranslated region |
| 195786 | 2.91E−04 | EST |
| 1048781 | 4.46E−02 | FLJ10140\|hypothetical protein FLJ10140 |
| 786213 | 3.97E−02 | AUH\|AU RNA-binding protein/enoyl-Coenzyme A hydratase |
| 66931 | 2.12E−02 | FLJ20307\|hypothetical protein FLJ20307 |
| 79898 | 4.71E−02 | TLE1\|transducin-like enhancer of split 1, homolog of Drosophila E(sp1) |
| 115292 | 1.66E−03 | DKFZp586C1924\|hypothetical protein DKFZp586C1924 |
| 360778 | 6.76E−05 | ATM\|ataxia telangiectasia mutated (includes complementation groups A, C and D) |
| 1732033 | 3.39E−02 | FLJ14427\|hypothetical protein FLJ14427 |
| 308163 | 3.45E−02 | ESTs, Weakly similar to TRHY_HUMAN TRICHOHYALI [H. sapiens] |
| 951068 | 2.97E−02 | Homo sapiens, clone IMAGE: 3450973, mRNA |
| 321945 | 3.96E−03 | ESTs |
| 897153 | 3.64E−02 | PTD009\|PTD009 protein |
| 150137 | 1.40E−02 | DKFZP564O123\|DKFZP564O123 protein |
| 610103 | 3.78E−02 | DKFZP434N1511\|hypothetical protein |
| 124261 | 2.36E−02 | SNRP70\|small nuclear ribonucleoprotein 70 kD polypeptide (RNP antigen) |
| 1926575 | 1.34E−02 | CDX2\|caudal type homeo box transcription factor 2 |
| 77361 | 3.57E−02 | LOC51119\|CGI-97 protein |
| 767641 | 1.34E−02 | MAPK8IP2\|mitogen-activated protein kinase 8 interacting protein 2 |
| 1610546 | 4.45E−04 | HNF3A\|hepatocyte nuclear factor 3, alpha |
| 502446 | 2.22E−02 | DKFZP564A2416\|DKFZP564A2416 protein |
| 490449 | 1.86E−02 | RAD50\|RAD50 (S. cerevisiae) homolog |
| 2014888 | 2.50E−02 | SRPUL\|sushi-repeat protein |
| 163174 | 3.21E−02 | TCEA1\|transcription elongation factor A (SII), 1 |
| 471863 | 2.31E−02 | Homo sapiens mRNA; cDNA DKFZp586C1817 (from clone DKFZp586C1817) |
| 753743 | 8.91E−03 | IL6ST\|interleukin 6 signal transducer (gp130, oncostatin M receptor) |
| 768520 | 4.09E−02 | NCALD\|neurocalcin delta |
| 1516938 | 3.55E−02 | HM74\|putative chemokine receptor; GTP-binding protein |
| 811941 | 4.96E−02 | Homo sapiens cDNA FLJ32130 fis, clone PEBLM2000248, weakly similar to ZINC FINGER PROTEIN 157 |
| 811944 | 1.41E−02 | ESTs |
| 298862 | 1.27E−03 | ESTs |
| 730953 | 1.36E−02 | FLJ13171\|hypothetical protein FLJ13171 |
| 770801 | 1.20E−02 | ESTs |
| 2010684 | 1.85E−02 | KIAA0640\|SWAP-70 protein |
| 712166 | 4.91E−02 | KIAA0855\|golgin-67 |
| 594172 | 2.44E−02 | Homo sapiens, clone MGC: 24302 IMAGE: 3996246, mRNA, complete cds |
| 26314 | 1.36E−02 | STXBP3\|syntaxin binding protein 3 |
| 128493 | 1.16E−02 | MLH1\|mutL (E. coli) homolog 1 (colon cancer, nonpolyposis type 2) |
| 1519341 | 1.04E−02 | KIAA0907\|KIAA0907 protein |
| 753754 | 2.06E−03 | ESTs |
| 26171 | 1.44E−02 | KIAA0856\|KIAA0856 protein |
| 1607482 | 4.52E−02 | CEBPG\|CCAAT/enhancer binding protein (C/EBP), gamma |
| 814350 | 3.80E−02 | IDE\|insulin-degrading enzyme |
| 796946 | 1.41E−02 | CSPG6\|chondroitin sulfate proteoglycan 6 (bamacan) |
| 344837 | 3.93E−02 | ESTs |
| 814285 | 4.45E−04 | FLJ11240\|hypothetical protein FLJ11240 |
| 156043 | 3.81E−02 | Homo sapiens cDNA: FLJ21933 fis, clone HEP04337 |
| 137602 | 1.56E−02 | Homo sapiens mRNA; cDNA DKFZp434G0972 (from clone DKFZp434G0972) |
| 322914 | 9.11E−03 | ACP1\|acid phosphatase 1, soluble |
| 366830 | 3.22E−02 | ESTs |
| 357940 | 4.24E−03 | FLJ22643\|hypothetical protein FLJ22643 |
| 898058 | 3.68E−02 | ESTs |
| 132452 | 4.87E−02 | ESTs |
| 343974 | 1.87E−02 | FLJ23445\|hypothetical protein FLJ23445 |
| 293001 | 3.20E−03 | DKFZP434E2318\|hypothetical protein DKFZp434E2318 |
| 782047 | 1.93E−02 | KIAA0268\|KIAA0268 protein |
| 767747 | 2.73E−02 | KIAA0999\|KIAA0999 protein |
| 1558268 | 1.67E−02 | PTMS\|parathymosin |
| 277761 | 5.24E−03 | ESTs |
| 150314 | 2.64E−02 | LYPLA1\|lysophospholipase I |
| 2051352 | 3.01E−02 | KLHL2\|kelch (Drosophila)-like 2 (Mayven) |
| 241798 | 2.20E−02 | Homo sapiens cDNA FLJ30407 fis, clone BRACE2008553 |
| 79216 | 3.76E−02 | AHNAK\|AHNAK nucleoprotein (desmoyokin) |
| 744952 | 1.97E−02 | ESTs, Moderately similar to UQHUR7 ubiquitin/ribosomal protein S27a, cytosolic [H. sapiens] |
| 292068 | 1.20E−02 | ESTs |
| 2018332 | 3.78E−02 | PRKAR1A\|protein kinase, cAMP-dependent, regulatory, type I, alpha (tissue specific extinguisher 1) |
| 592592 | 1.50E−02 | MUC5AC\|mucin 5, subtypes A and C, tracheobronchial/gastric |
| 133197 | 2.82E−02 | KIAA0997\|KIAA0997 protein |
| 563451 | 3.20E−03 | TLK1\|tousled-like kinase 1 |
| 811032 | 2.11E−02 | PAWR\|PRKC, apoptosis, WT1, regulator |
| 786194 | 2.07E−02 | DCK\|deoxycytidine kinase |
| 767753 | 4.53E−03 | RFX5\|regulatory factor X, 5 (influences HLA class II expression) |
| 595070 | 1.49E−03 | SERP1\|stress-associated endoplasmic reticulum protein 1; ribosome associated membrane protein 4 |
| 770835 | 1.04E−02 | BCKDHB\|branched chain keto acid dehydrogenase E1, beta polypeptide (maple syrup urine disease) |
| 277848 | 3.73E−02 | Homo sapiens cDNA FLJ13900 fis, clone THYRO1001746 |
| 428184 | 1.78E−02 | Homo sapiens, clone MGC: 18216 IMAGE: 4156235, mRNA, complete cds |
| 207989 | 2.58E−04 | KIAA0022\|KIAA0022 gene product |
| 857640 | 1.12E−02 | COL6A2\|collagen, type VI, alpha 2 |
| 1894519 | 1.13E−02 | FLJ12085\|hypothetical protein FLJ12085 |
| 950603 | 1.31E−03 | Homo sapiens clone 24670 mRNA sequence |
| 223304 | 1.02E−02 | ESTs |
| 365990 | 1.14E−02 | Homo sapiens cDNA FLJ11567 fis, clone HEMBA1003276 |
| 770848 | 4.41E−02 | ESTs, Weakly similar to ALU1_HUMAN ALU SUBFAMILY J SEQUENCE CONTAMINATION WARNING ENTRY |
| | | [H. sapiens] |
| 193383 | 1.13E−02 | FLJ20986\|hypothetical protein FLJ20986 |
| 1762326 | 2.03E−02 | ESTs |
| 263955 | 3.39E−02 | KIAA0828\|KIAA0828 protein |
| 82171 | 2.11E−02 | Homo sapiens cDNA FLJ14041 fis, clone HEMBA1005780 |
| 487499 | 2.11E−02 | Homo sapiens cDNA FLJ32068 fis, clone OCBBF1000114 |
| 1568056 | 3.84E−02 | ESTs, Moderately similar to I78885 serine/threonine-specific protein kinase [H. sapiens] |
| 260619 | 1.33E−02 | USP12\|ubiquitin specific protease 12 |
| 1732247 | 8.78E−03 | ESTs |
| 845355 | 1.78E−02 | CTSC\|cathepsin C |
| 1422894 | 9.53E−03 | NOTCH2\|Notch (Drosophila) homolog 2 |
| 428411 | 4.45E−04 | KIAA1915\|KIAA1915 protein |
| 136845 | 2.11E−02 | Homo sapiens, clone IMAGE: 3915000, mRNA |
| 142259 | 3.88E−02 | FIP2\|tumor necrosis factor alpha-inducible cellular protein containing leucine zipper domains; Huntingtin interacting |
| | | protein L; transcrption factor IIIA-interacting protein |
| 788109 | 4.64E−03 | ATR\|ataxia telangiectasia and Rad3 related |
| 114852 | 1.82E−02 | C16orf3\|chromosome 16 open reading frame 3 |
| 784830 | 4.32E−02 | D123\|D123 gene product |
| 2009477 | 2.11E−02 | CD6\|CD6 antigen |

TABLE 3
Genes, the expressions of which positively correlate with the ERb subtype

| Clone_ID | P_value | Gene_Description |
|---|---|---|
| 898312 | 4.87E−02 | TRAF4\|TNF receptor-associated factor 4 |
| 2713047 | 3.35E−02 | PVR\|poliovirus receptor |
| 739511 | 6.40E−03 | PKMYT1\|membrane-associated tyrosine- and threonine-specific cdc2-inhibitory kinase |
| 323693 | 2.69E−02 | AP1S1\|adaptor-related protein complex 1, sigma 1 subunit |
| 29927 | 1.14E−02 | FLJ10737\|hypothetical protein FLJ10737 |
| 770935 | 2.18E−02 | 7h3\|hypothetical protein FLJ13511 |
| 1681421 | 3.88E−03 | EGFL3\|EGF-like-domain, multiple 3 |
| 50649 | 3.71E−02 | PRKCL1\|protein kinase C-like 1 |
| 203003 | 3.93E−02 | NME4\|non-metastatic cells 4, protein expressed in |
| 795263 | 1.58E−02 | FLJ22638\|hypothetical protein FLJ22638 |
| 731020 | 4.17E−02 | PSMF1\|proteasome (prosome, macropain) inhibitor subunit 1 (PI31) |
| 1460075 | 1.20E−02 | PIN1\|protein (peptidyl-prolyl cis/trans isomerase) NIMA-interacting 1 |
| 108377 | 1.22E−02 | TUBG1\|tubulin, gamma 1 |
| 727078 | 4.92E−03 | Homo sapiens cDNA: FLJ23602 fis, clone LNG15735 |
| 740788 | 1.80E−02 | ESTs, Weakly similar to CA13 MOUSE COLLAGEN ALPHA 1(III) CHAIN PRECURSOR [M. musculus] |
| 756502 | 2.05E−03 | NUDT1\|nudix (nucleoside diphosphate linked moiety X)-type motif 1 |
| 53122 | 3.45E−02 | Human (clone CTG-A4) mRNA sequence |
| 1903066 | 8.90E−03 | KRTHB1\|keratin, hair, basic, 1 |
| 753021 | 2.95E−02 | NOSIP\|eNOS interacting protein |
| 841308 | 4.45E−03 | MYLK\|myosin, light polypeptide kinase |
| 144887 | 4.86E−02 | DPM2\|dolichyl-phosphate mannosyltransferase polypeptide 2, regulatory subunit |
| 866712 | 2.67E−03 | MGC14421\|hypothetical protein MGC14421 |
| 2019258 | 3.40E−02 | ESTs |
| 743268 | 4.03E−02 | MGC2835\|hypothetical protein MGC2835 |
| 796079 | 2.24E−04 | MGC4171\|hypothetical protein MGC4171 |
| 154720 | 8.98E−03 | ARD1\|N-acetyltransferase, homolog of S. cerevisiae ARD1 |
| 324651 | 4.44E−02 | LOC51102\|CGI-63 protein |
| 725558 | 3.84E−02 | LOC51114\|CGI-89 protein |
| 366100 | 4.39E−02 | MATN2\|matrilin 2 |
| 51604 | 5.33E−03 | RLUCL\|ribosomal large subunit pseudouridine synthase C like |
| 756372 | 9.48E−03 | RARRES2\|retinoic acid receptor responder (tazarotene induced) 2 |
| 756373 | 2.51E−03 | ARHGEF16\|Rho guanine exchange factor (GEF) 16 |
| 770884 | 1.97E−02 | TIP-1\|Tax interaction protein 1 |
| 591994 | 3.71E−02 | FLJ21935\|hypothetical protein FLJ21935 |
| 2018392 | 2.60E−02 | GLIS2\|Kruppel-like zinc finger protein GLIS2 |
| 813841 | 3.88E−02 | PLAT\|plasminogen activator, tissue |
| 788209 | 1.29E−02 | FLJ11807\|hypothetical protein FLJ11807 |
| 727164 | 1.30E−02 | MGC13114\|hypothetical protein MGC13114 |
| 262251 | 8.91E−03 | CLCN7\|chloride channel 7 |
| 502753 | 2.16E−02 | ANGPT2\|angiopoietin 2 |
| 502682 | 3.28E−02 | ENIGMA\|enigma (LIM domain protein) |
| 1409509 | 2.11E−02 | TNNT1\|troponin T1, skeletal, slow |
| 138550 | 2.11E−02 | FLJ11137\|hypothetical protein FLJ11137 |
| 139354 | 1.97E−02 | HSPC195\|hypothetical protein |
| 126320 | 4.54E−02 | JUP\|junction plakoglobin |
| 195313 | 4.28E−02 | KPNA6\|karyopherin alpha 6 (importin alpha 7) |
| 1323361 | 1.53E−02 | NR2F6\|nuclear receptor subfamily 2, group F, member 6 |
| 1473274 | 1.31E−02 | MYRL2\|myosin regulatory light chain 2, smooth muscle isoform |
| 2028161 | 3.45E−02 | UNC93B\|unc93 (C. elegans) homolog B |
| 433204 | 2.58E−04 | Homo sapiens, Similar to RIKEN cDNA 2310012N15 gene, clone IMAGE: 3342825, mRNA, partial cds |
| 1917207 | 1.77E−02 | HIG2\|hypoxia-inducible protein 2 |
| 753984 | 1.34E−02 | FLJ10640\|hypothetical protein |
| 809974 | 2.15E−02 | ESTs, Weakly similar to S10889 proline-rich protein [H. sapiens] |
| 1568318 | 1.07E−02 | DNASE1\|deoxyribonuclease I |
| 80764 | 4.35E−03 | LOC51255\|hypothetical protein |
| 769565 | 3.51E−02 | RER1\|similar to S. cerevisiae RER1 |
| 39722 | 7.38E−03 | ERCC2\|excision repair cross-complementing rodent repair deficiency, complementation group 2 (xeroderma |
| | | pigmentosum D) |
| 49273 | 6.00E−03 | SLC27A4\|solute carrier family 27 (fatty acid transporter), member 4 |
| 1600239 | 4.03E−02 | LOC51659\|HSPC037 protein |
| 135221 | 4.63E−02 | S100P\|S100 calcium-binding protein P |
| 898281 | 4.25E−02 | FLNA\|filamin A, alpha (actin-binding protein-280) |
| 841334 | 2.91E−03 | STIP1\|stress-induced-phosphoprotein 1 (Hsp70/Hsp90-organizing protein) |
| 2027515 | 2.58E−04 | SFN\|stratifin |
| 1323448 | 4.90E−02 | CRIP1\|cysteine-rich protein 1 (intestinal) |
| 591143 | 1.44E−02 | LOC51329\|SRp25 nuclear protein |
| 2017821 | 3.78E−05 | NTHL1\|nth (E. coli endonuclease III)-like 1 |
| 1968422 | 4.59E−02 | Homo sapiens mRNA full length insert cDNA clone EUROIMAGE 1968422 |
| 841338 | 1.31E−02 | PRNPIP\|prion protein interacting protein |
| 1473289 | 8.98E−03 | PPGB\|protective protein for beta-galactosidase (galactosialidosis) |
| 815535 | 2.03E−03 | TCOF1\|Treacher Collins-Franceschetti syndrome 1 |
| 2017754 | 4.22E−03 | DGSI\|DiGeorge syndrome critical region gene DGSI; likely ortholog of mouse expressed sequence |
| | | 2 embryonic lethal |
| 121251 | 2.29E−02 | MGC5576\|hypothetical protein MGC5576 |
| 769712 | 3.00E−02 | GAK\|cyclin G associated kinase |
| 66406 | 3.82E−02 | ESTs, Highly similar to T47163 hypothetical protein DKFZp762E1312.1 [H. sapiens] |
| 73550 | 2.91E−04 | FLJ11773\|hypothetical protein FLJ11773 |
| 2015148 | 9.48E−03 | GIT1\|G protein-coupled receptor kinase-interactor 1 |
| 767034 | 2.02E−03 | ILVBL\|ilvB (bacterial acetolactate synthase)-like |
| 714159 | 1.51E−03 | Homo sapiens cDNA FLJ32185 fis, clone PLACE6001925 |
| 770043 | 2.58E−04 | NDUFV1\|NADH dehydrogenase (ubiquinone) flavoprotein 1 (51 kD) |
| 1642496 | 3.82E−02 | MGC11266\|hypothetical protein MGC11266 |
| 795522 | 4.96E−02 | TAF1C\|TATA box binding protein (TBP)-associated factor, RNA polymerase I, C, 110 kD |
| 221846 | 4.57E−02 | CHES1\|checkpoint suppressor 1 |
| 50768 | 2.89E−02 | DKFZp667O2416\|hypothetical protein DKFZp667O2416 |
| 68950 | 1.77E−02 | CCNE1\|cyclin E1 |
| 130153 | 1.66E−02 | SUPT5H\|suppressor of Ty (S. cerevisiae) 5 homolog |
| 338599 | 4.09E−02 | NRBP\|nuclear receptor binding protein |
| 1859037 | 2.38E−02 | DKFZP586J0119\|DKFZP586J0119 protein |
| 138728 | 4.91E−02 | KIAA1696\|KIAA1696 protein |
| 897570 | 1.77E−02 | TRAP1\|heat shock protein 75 |
| 471266 | 1.40E−02 | DGCR6L\|DiGeorge syndrome critical region gene 6 like |
| 240367 | 1.22E−02 | CTCF\|CCCTC-binding factor (zinc finger protein) |
| 1635286 | 4.40E−03 | ITGB4BP\|integrin beta 4 binding protein |
| 179163 | 4.87E−03 | GRIN2C\|glutamate receptor, ionotropic, N-methyl D-aspartate 2C |
| 840556 | 1.93E−02 | EIF4EL3\|eukaryotic translation initiation factor 4E-like 3 |
| 755689 | 1.41E−02 | RARG\|retinoic acid receptor, gamma |
| 788185-2 | 4.35E−02 | TNFRSF10B\|tumor necrosis factor receptor superfamily, member 10b |
| 346696 | 8.98E−03 | TEAD4\|TEA domain family member 4 |
| 725672 | 2.58E−04 | Homo sapiens, Similar to transducin (beta)-like 3, clone MGC: 8613 IMAGE: 2961321, mRNA, complete cds |
| 81662 | 4.35E−02 | PTD004\|hypothetical protein |
| 785847 | 3.39E−02 | UBE2M\|ubiquitin-conjugating enzyme E2M (homologous to yeast UBC12) |
| 1635364 | 4.52E−02 | LSM2\|U6 snRNA-associated Sm-like protein |
| 809939-2 | 3.34E−02 | MAPK3\|mitogen-activated protein kinase 3 |
| 44292 | 2.92E−02 | Homo sapiens mRNA; cDNA DKFZp434C107 (from clone DKFZp434C107) |
| 753153 | 8.88E−03 | IL13RA1\|interleukin 13 receptor, alpha 1 |
| 2019526 | 4.62E−02 | FLJ14220\|hypothetical protein FLJ14220 |
| 68103 | 3.30E−02 | MLC1SA\|myosin light chain 1 slow a |
| 265853 | 1.94E−03 | TEM8\|tumor endothelial marker 8 |
| 1470048 | 5.20E−03 | LY6E\|lymphocyte antigen 6 complex, locus E |
| 743536 | 3.62E−02 | EST |
| 823727 | 3.17E−02 | Homo sapiens, clone IMAGE: 2905978, mRNA, partial cds |
| 249672 | 3.30E−02 | FLJ12827\|hypothetical protein FLJ12827 |
| 2019387 | 4.54E−02 | SNAPC4\|small nuclear RNA activating complex, polypeptide 4, 190 kD |
| 2519200 | 4.03E−02 | LY6H\|lymphocyte antigen 6 complex, locus H |
| 1522696 | 4.80E−02 | FLJ10850\|hypothetical protein FLJ10850 |
| 47853 | 4.35E−02 | ALDH4A1\|aldehyde dehydrogenase 4 family, member A1 |
| 138672 | 4.85E−02 | ESTs |
| 35620 | 1.16E−03 | MGC4707\|hypothetical protein MGC4707 |
| 26806 | 1.97E−02 | MGC10433\|hypothetical protein MGC10433 |
| 1669672 | 2.72E−02 | THY1\|Thy-1 cell surface antigen |
| 826138 | 3.80E−02 | GAMT\|guanidinoacetate N-methyltransferase |
| 1612722 | 1.90E−02 | FLJ20542\|hypothetical protein FLJ20542 |
| 1703339 | 3.80E−02 | STXBP2\|syntaxin binding protein 2 |
| 171912 | 2.24E−04 | Homo sapiens mRNA full length insert cDNA clone EUROIMAGE 703547 |
| 430928 | 3.64E−02 | BARD1\|BRCA1 associated RING domain 1 |
| 235923 | 3.01E−04 | DKFZP434P1750\|DKFZP434P1750 protein |
| 812238 | 1.93E−02 | MGC4692\|hypothetical protein MGC4692 |
| 2013659 | 3.22E−02 | FLJ20294\|hypothetical protein FLJ20294 |
| 1654978 | 3.51E−02 | FLJ22504\|hypothetical C2H2 zinc finger protein FLJ22504 |
| 366315 | 4.37E−03 | Homo sapiens, clone MGC: 20500 IMAGE: 4053084, mRNA, complete cds |
| 714196 | 3.10E−02 | WDR1\|WD repeat domain 1 |
| 897745 | 1.12E−02 | FLJ13868\|hypothetical protein FLJ13868 |
| 128126 | 2.01E−02 | DAF\|decay accelerating factor for complement (CD55, Cromer blood group system) |
| 60565 | 1.12E−02 | LLGL2\|lethal giant larvae (Drosophila) homolog 2 |
| 1142132 | 3.01E−02 | RPIP8\|RaP2 interacting protein 8 |
| 1535957 | 1.58E−02 | SEC6\|similar to S. cerevisiae Sec6p and R. norvegicus rsec6 |
| 487882 | 2.42E−03 | DKFZP761D0211\|hypothetical protein DKFZp761D0211 |
| 360436 | 1.42E−02 | COPEB\|core promoter element binding protein |
| 1592715 | 1.95E−02 | HOMER-3\|Homer, neuronal immediate early gene, 3 |
| 1845169 | 2.91E−03 | RAB35\|RAB35, member RAS oncogene family |
| 741954 | 3.83E−02 | Homo sapiens cDNA FLJ14656 fis, clone NT2RP2002439 |
| 812170 | 4.73E−02 | KIAA0657\|KIAA0657 protein |
| 166236 | 4.31E−03 | 2.19\|2.19 gene |
| 714414 | 2.44E−02 | UQCRC1\|ubiquinol-cytochrome c reductase core protein I |
| 772912 | 7.87E−03 | AGS3\|likely ortholog of rat activator of G-protein signaling 3 |
| 1557018 | 9.48E−03 | C21orf70\|chromosome 21 open reading frame 70 |
| 235938 | 1.66E−03 | BAK1\|BCL2-antagonist/killer 1 |
| 1632120 | 1.70E−02 | COPE\|coatomer protein complex, subunit epsilon |
| 2322079 | 7.56E−03 | EST |
| 358162 | 4.30E−02 | HSU79266\|protein predicted by clone 23627 |
| 756666 | 1.09E−03 | PPP1CA\|protein phosphatase 1, catalytic subunit, alpha isoform |
| 32231 | 1.34E−02 | FLJ12442\|hypothetical protein FLJ12442 |
| 346942 | 2.98E−02 | PIGQ\|phosphatidylinositol glycan, class Q |
| 531319 | 8.42E−03 | STK12\|serine/threonine kinase 12 |
| 2027578 | 1.85E−02 | NAKAP95\|neighbor of A-kinase anchoring protein 95 |
| 741891 | 4.61E−02 | RAB2L\|RAB2, member RAS oncogene family-like |
| 814865 | 8.91E−03 | MGC11102\|hypothetical protein MGC11102 |
| 1569187 | 3.53E−02 | HS3ST4\|heparan sulfate (glucosamine) 3-O-sulfotransferase 4 |
| 2623626 | 3.98E−02 | PTPRG\|protein tyrosine phosphatase, receptor type, G |
| 49485 | 8.04E−04 | Homo sapiens, clone IMAGE: 3161564, mRNA, partial cds |
| 1555427 | 1.93E−02 | SPINT1\|serine protease inhibitor, Kunitz type 1 |
| 780947 | 1.14E−02 | POLD1\|polymerase (DNA directed), delta 1, catalytic subunit (125 kD) |
| 455275 | 3.81E−02 | FLJ23469\|hypothetical protein FLJ23469 |
| 209066-2 | 3.42E−02 | STK15\|serine/threonine kinase 15 |
| 1759582 | 4.40E−03 | FN14\|type I transmembrane protein Fn14 |
| 141852 | 3.68E−02 | P2RY2\|purinergic receptor P2Y, G-protein coupled, 2 |
| 897768 | 4.25E−02 | COL7A1\|collagen, type VII, alpha 1 (epidermolysis bullosa, dystrophic, dominant and recessive) |
| 41208 | 1.29E−03 | BMP1\|bone morphogenetic protein 1 |
| 825293 | 3.11E−02 | KIAA0082\|KIAA0082 protein |
| 1860497 | 2.19E−02 | Homo sapiens, clone MGC: 5352 IMAGE: 3048106, mRNA, complete cds |
| 344272 | 2.02E−02 | EMP3\|epithelial membrane protein 3 |
| 327506 | 1.87E−02 | Homo sapiens mRNA full length insert cDNA clone EUROIMAGE 327506 |
| 430954 | 1.84E−02 | FLJ22341\|hypothetical protein FLJ22341 |
| 260015 | 7.21E−03 | DKFZP586B0519\|DKFZP586B0519 protein |
| 2017897 | 3.67E−02 | CINP\|HeLa cyclin-dependent kinase 2 interacting protein |
| 431759 | 4.39E−02 | TEAD3\|TEA domain family member 3 |
| 810734 | 3.01E−03 | POLD4\|polymerase (DNA-directed), delta 4 |
| 357450 | 1.30E−02 | MTVR\|Mouse Mammary Turmor Virus Receptor homolog |
| 897770 | 3.34E−03 | EST |
| 26910 | 4.00E−02 | T54\|T54 protein |
| 897774 | 7.38E−03 | APRT\|adenine phosphoribosyltransferase |
| 1536925 | 1.70E−02 | PDPK1\|3-phosphoinositide dependent protein kinase-1 |
| 207618 | 1.34E−02 | ARAF1\|v-raf murine sarcoma 3611 viral oncogene homolog 1 |
| 756687 | 2.02E−02 | CD36L1\|CD36 antigen (collagen type I receptor, thrombospondin receptor)-like 1 |
| 1588935 | 4.27E−02 | PHLDA3\|pleckstrin homology-like domain, family A, member 3 |
| 742783 | 1.66E−03 | DKFZp434N035\|hypothetical protein DKFZp434N035 |
| 172751 | 1.97E−02 | APBA1\|amyloid beta (A4) precursor protein-binding, family A, member 1 (X11) |
| 562080 | 3.04E−04 | FLJ10101\|hypothetical protein FLJ10101 |
| 810743 | 9.21E−03 | MLF2\|myeloid leukemia factor 2 |
| 166268 | 4.20E−02 | SR-A1\|serine arginine-rich pre-mRNA splicing factor SR-A1 |
| 1476053 | 1.12E−02 | RAD51\|RAD51 (S. cerevisiae) homolog (E coli RecA homolog) |
| 1947381 | 2.47E−02 | FLJ22329\|hypothetical protein FLJ22329 |
| 1731860 | 4.47E−02 | GADD45B\|growth arrest and DNA-damage-inducible, beta |
| 2062432 | 4.88E−03 | COMP\|cartilage oligomeric matrix protein (pseudoachondroplasia, epiphyseal dysplasia 1, multiple) |
| 128302 | 2.16E−02 | PTMS\|parathymosin |
| 593114 | 4.44E−02 | SIPA1\|signal-induced proliferation-associated gene 1 |
| 897781 | 3.10E−02 | KRT8\|keratin 8 |
| 843091 | 1.73E−02 | MGC20533\|similar to RIKEN cDNA 2410004L22 gene (M. musculus) |
| 611532 | 8.98E−03 | TNNI2\|troponin I, skeletal, fast |
| 590640 | 2.24E−04 | PDXK\|pyridoxal (pyridoxine, vitamin B6) kinase |
| 809413 | 1.28E−03 | FLJ12875\|hypothetical protein FLJ12875 |
| 878406 | 3.75E−02 | MTX1\|metaxin 1 |
| 26856 | 2.59E−02 | FLOT2\|flotillin 2 |
| 814961 | 4.96E−02 | USP5\|ubiquitin specific protease 5 (isopeptidase T) |
| 840698 | 2.10E−03 | FLJ20254\|hypothetical protein FLJ20254 |
| 2009969 | 1.51E−02 | 20D7-FC4\|hypothetical protein |
| 1610168 | 2.67E−03 | DMWD\|dystrophia myotonica-containing WD repeat motif |
| 41302 | 2.69E−02 | KIAA0643\|KIAA0643 protein |
| 307069 | 1.93E−02 | ALDH3B1\|aldehyde dehydrogenase 3 family, member B1 |
| 878413 | 1.70E−02 | SLC25A11\|solute carrier family 25 (mitochondrial carrier; oxoglutarate carrier), member 11 |
| 267590 | 4.70E−02 | KIAA0330\|calcineurin binding protein 1 |
| 302996 | 4.50E−04 | CLIC3\|chloride intracellular channel 3 |
| 884692 | 2.74E−03 | TCEB2\|transcription elongation factor B (SIII), polypeptide 2 (18 kD, elongin B) |
| 259579 | 2.61E−02 | RAD51L3\|RAD51 (S. cerevisiae)-like 3 |
| 859761 | 2.68E−02 | PVRL2\|poliovirus receptor-related 2 (herpesvirus entry mediator B) |
| 825399 | 4.52E−02 | TRAF3\|TNF receptor-associated factor 3 |
| 74738 | 9.83E−03 | MGC20486\|hypothetical protein MGC20486 |
| 768217 | 2.19E−02 | Homo sapiens, Similar to hypothetical protein, MGC: 7764, clone MGC: 20548 IMAGE: 3607345, mRNA, |
| | | complete cds |
| 811565 | 1.41E−03 | KIAA1694\|KIAA1694 protein |
| 843321 | 1.97E−02 | KRT7\|keratin 7 |
| 294273 | 9.39E−03 | PXMP2\|peroxisomal membrane protein 2 (22 kD) |
| 809503 | 3.20E−02 | ESTs, Weakly similar to AC004858 3 U1 small ribonucleoprotein 1SNRP homolog [H. sapiens] |
| 1609781 | 9.51E−03 | Homo sapiens clone 24819 mRNA sequence |
| 780989 | 4.09E−02 | DKFZP434N061\|DKFZP434N061 protein |
| 526757 | 1.14E−02 | CCND1\|cyclin D1 (PRAD1: parathyroid adenomatosis 1) |
| 1632247 | 3.38E−02 | FLJ23436\|hypothetical protein FLJ23436 |
| 2018941 | 1.09E−03 | D21S2056E\|DNA segment on chromosome 21 (unique) 2056 expressed sequence |
| 809507 | 2.06E−03 | FLJ20568\|hypothetical protein FLJ20568 |
| 771089 | 1.07E−02 | NDUFB7\|NADH dehydrogenase (ubiquinone) 1 beta subcomplex, 7 (18 kD, B18) |
| 306575-2 | 1.22E−02 | DIPA\|hepatitis delta antigen-interacting protein A |
| 25069 | 1.97E−02 | KIAA0462\|KIAA0462 protein |
| 502151 | 8.37E−04 | SLC16A3\|solute carrier family 16 (monocarboxylic acid transporters), member 3 |
| 784260 | 3.05E−03 | MAN1B1\|mannosidase, alpha, class 1B, member 1 |
| 814989 | 3.46E−04 | PPM1G\|protein phosphatase 1G (formerly 2C), magnesium-dependent, gamma isoform |
| 377018 | 1.14E−02 | FLJ20850\|hypothetical protein FLJ20850 |
| 1574058 | 8.98E−03 | AGPAT2\|1-acylglycerol-3-phosphate O-acyltransferase 2 (lysophosphatidic acid acyltransferase, beta) |
| 235056 | 4.45E−03 | 24432\|hypothetical protein 24432 |
| 771233 | 5.17E−03 | Homo sapiens, clone MGC: 16395 IMAGE: 3939387, mRNA, complete cds |
| 291880 | 1.34E−02 | MFAP2\|microfibrillar-associated protein 2 |
| 809512 | 1.53E−02 | FLJ10767\|hypothetical protein FLJ10767 |
| 2125819 | 1.60E−02 | BAX\|BCL2-associated X protein |
| 1837280 | 9.08E−03 | EST |
| 346134 | 3.39E−02 | CRHSP-24\|calcium-regulated heat-stable protein (24 kD) |
| 1535082 | 4.39E−02 | KIAA1271\|KIAA1271 protein |
| 1470278 | 2.99E−02 | FLJ21841\|hypothetical protein FLJ21841 |
| 246704 | 1.23E−02 | RAI\|RelA-associated inhibitor |
| 1575008 | 3.48E−02 | WBP1\|WW domain binding protein 1 |
| 32299 | 3.34E−02 | IMPA2\|inositol(myo)-1(or 4)-monophosphatase 2 |
| 296030 | 2.32E−02 | Homo sapiens cDNA: FLJ20944 fis, clone ADSE01780 |
| 2315207 | 1.94E−02 | SCYB6\|small inducible cytokine subfamily B (Cys-X-Cys), member 6 (granulocyte chemotactic protein 2) |
| 1882823 | 2.73E−02 | ESTs |
| 810927 | 3.25E−03 | RFXANK\|regulatory factor X-associated ankyrin-containing protein |
| 838662 | 1.04E−02 | HCNGP\|transcriptional regulator protein |
| 2314197 | 3.36E−02 | FLJ12671\|hypothetical protein FLJ12671 |
| 809521 | 1.85E−02 | HMT-1\|beta-1,4 mannosyltransferase |
| 41406 | 4.52E−02 | NMA\|putative transmembrane protein |
| 796723 | 4.09E−02 | Homo sapiens clone CDABP0014 mRNA sequence |
| 1690762 | 2.60E−02 | CDK10\|cyclin-dependent kinase (CDC2-like) 10 |
| 1908666 | 3.81E−02 | ZNF79\|zinc finger protein 79 (pT7) |
| 788566 | 2.69E−02 | PCP4\|Purkinje cell protein 4 |
| 1732922 | 6.02E−03 | Homo sapiens mRNA; cDNA DKFZp762H106 (from clone DKFZp762H106) |
| 1492426 | 1.49E−02 | C19orf3\|chromosome 19 open reading frame 3 |
| 2010543 | 1.07E−02 | DDX28\|DEAD/H (Asp-Glu-Ala-Asp/His) box polypeptide 28 |
| 769986 | 3.75E−04 | NUBP2\|nucleotide binding protein 2 (E. coli MinD like) |
| 299388 | 4.44E−02 | PP15\|nuclear transport factor 2 (placental protein 15) |
| 2322367 | 4.55E−02 | RTN4\|reticulon 4 |
| 771323 | 1.33E−02 | PLOD\|procollagen-lysine, 2-oxoglutarate 5-dioxygenase (lysine hydroxylase, Ehlers-Danlos syndrome type VI) |
| 897107 | 9.38E−04 | SLC25A1\|solute carrier family 25 (mitochondrial carrier; citrate transporter), member 1 |
| 184240 | 8.88E−03 | ESTs |
| 1551282 | 3.57E−02 | FLJ13956\|hypothetical protein FLJ13956 |
| 124143 | 2.58E−03 | DKFZP761H1710\|hypothetical protein DKFZp761H1710 |
| 770388 | 2.73E−02 | CLDN4\|claudin 4 |
| 809609 | 2.08E−02 | Homo sapiens cDNA FLJ32583 fis, clone SPLEN2000348 |
| 815017 | 3.34E−02 | Homo sapiens HSPC337 mRNA, partial cds |
| 629916 | 2.19E−02 | TIM17B\|translocase of inner mitochondrial membrane 17 homolog B (yeast) |
| 1521341 | 8.91E−03 | HIRIP3\|HIRA-interacting protein 3 |
| 251330 | 1.14E−02 | MGC10540\|hypothetical protein MGC10540 |
| 510273 | 3.67E−02 | PLEC1\|plectin 1, intermediate filament binding protein, 500 kD |
| 810942 | 8.97E−03 | IDH3G\|isocitrate dehydrogenase 3 (NAD+) gamma |
| 1476251 | 7.10E−03 | FLJ20512\|hypothetical protein FLJ20512 |
| 810948 | 1.22E−02 | TRAP240\|thyroid hormone receptor-associated protein, 240 kDa subunit |
| 45632 | 2.99E−02 | GYS1\|glycogen synthase 1 (muscle) |
| 279146 | 8.91E−03 | ITPKC\|inositol 1,4,5-trisphosphate 3-kinase C |
| 753620 | 3.17E−02 | IGFBP6\|insulin-like growth factor binding protein 6 |
| 755228 | 2.54E−02 | DNM1\|dynamin 1 |
| 489076-2 | 2.61E−02 | EMILIN\|elastin microfibril interface located protein |
| 347035 | 4.03E−02 | KIAA0476\|KIAA0476 gene product |
| 1850224 | 1.99E−02 | ESTs |
| 825583 | 3.91E−04 | RALY\|RNA-binding protein (autoantigenic) |
| 742125 | 2.23E−02 | LOXL1\|lysyl oxidase-like 1 |
| 504945 | 3.75E−04 | FLJ20608\|hypothetical protein FLJ20608 |
| 1947804 | 1.93E−02 | TREX1\|three prime repair exonuclease 1 |
| 1699142 | 1.53E−02 | AP1G2\|adaptor-related protein complex 1, gamma 2 subunit |
| 343695 | 1.67E−02 | Homo sapiens cDNA FLJ31668 fis, clone NT2RI2004916 |
| 1506046 | 1.74E−02 | FLJ10815\|hypothetical protein FLJ10815 |
| 855749 | 4.28E−02 | TPI1\|triosephosphate isomerase 1 |
| 269606 | 2.02E−02 | MPG\|N-methylpurine-DNA glycosylase |
| 739993 | 4.54E−02 | BRE\|brain and reproductive organ-expressed (TNFRSF1A modulator) |
| 183602 | 5.77E−03 | KRT14\|keratin 14 (epidermolysis bullosa simplex, Dowling-Meara, Koebner) |
| 183462 | 3.48E−02 | MAN2C1\|mannosidase, alpha, class 2C, member 1 |
| 809557 | 9.15E−03 | MCM3\|minichromosome maintenance deficient (S. cerevisiae) 3 |
| 725224 | 2.79E−02 | HES6\|likely ortholog of mouse Hes6 neuronal differentiation gene |
| 564981 | 9.30E−03 | Homo sapiens, Similar to RIKEN cDNA 2810433K01 gene, clone MGC: 10200 IMAGE: 3909951, mRNA, |
| | | complete cds |
| 811907 | 1.06E−02 | FLJ22056\|hypothetical protein FLJ22056 |
| 323522 | 2.98E−02 | NRBP\|nuclear receptor binding protein |
| 951117 | 4.34E−02 | SHMT2\|serine hydroxymethyltransferase 2 (mitochondrial) |
| 511096 | 4.96E−03 | Homo sapiens, Similar to RIKEN cDNA 2010317E24 gene, clone IMAGE: 3502019, mRNA, partial cds |
| 502277 | 4.05E−02 | LOC51025\|CGI-136 protein |
| 700900 | 4.90E−02 | LOC51693\|unknown |
| 625584 | 3.59E−02 | TRIP\|TRAF interacting protein |
| 37708 | 2.68E−02 | MGC3101\|hypothetical protein MGC3101 |
| 2508044 | 1.49E−02 | HP\|haptoglobin |
| 150118 | 2.70E−02 | DKFZp434F054\|hypothetical protein DKFZp434F054 |
| 2018131 | 2.11E−02 | RACGAP1\|Rac GTPase activating protein 1 |
| 813514 | 4.12E−02 | FLJ22573\|hypothetical protein FLJ22573 |
| 700699 | 6.02E−03 | IL1RL1LG\|putative T1/ST2 receptor binding protein |
| 796694 | 1.80E−02 | BIRC5\|baculoviral IAP repeat-containing 5 (survivin) |
| 138672-2 | 4.54E−02 | ESTs |
| 811848 | 2.06E−02 | LOC56912\|hypothetical protein |
| 1492463 | 2.42E−03 | SEPX1\|selenoprotein X, 1 |
| 1947827 | 2.95E−02 | MSTP028\|MSTP028 protein |
| 839583 | 3.71E−02 | ESTs, Moderately similar to T46386 hypothetical protein DKFZp434P011.1 [H. sapiens] |
| 810979 | 2.91E−03 | MRPS2\|mitochondrial ribosomal protein S2 |
| 712139 | 3.45E−02 | ARL7\|ADP-ribosylation factor-like 7 |
| 592540 | 2.86E−02 | KRT5\|keratin 5 (epidermolysis bullosa simplex, Dowling-Meara/Kobner/Weber-Cockayne types) |
| 2019011 | 6.76E−05 | MT3\|metallothionein 3 (growth inhibitory factor (neurotrophic)) |
| 241677 | 6.64E−03 | MGC15416\|hypothetical protein MGC15416 |
| 770709 | 2.42E−02 | KIAA1089\|KIAA1089 protein |
| 740620 | 1.20E−02 | TPM2\|tropomyosin 2 (beta) |
| 882515 | 3.34E−02 | EIF3S9\|eukaryotic translation initiation factor 3, subunit 9 (eta, 116 kD) |
| 1574330 | 3.11E−02 | GROS1\|growth suppressor 1 |
| 503234 | 8.91E−03 | FLJ23471\|hypothetical protein FLJ23471 |
| 811923 | 1.07E−02 | POLE\|polymerase (DNA directed), epsilon |
| 1592048 | 1.70E−02 | SSNA1\|Sjogrens syndrome nuclear autoantigen 1 |
| 810983 | 1.37E−02 | DKFZP434H132\|DKFZP434H132 protein |
| 462961 | 2.17E−02 | DHFR\|dihydrofolate reductase |
| 839594 | 4.20E−02 | LTBP1\|latent transforming growth factor beta binding protein 1 |
| 1534633 | 1.03E−03 | MGC2479\|hypothetical protein MGC2479 |
| 770579 | 1.12E−02 | CLDN3\|claudin 3 |
| 184362 | 2.49E−02 | KCNJ9\|potassium inwardly-rectifying channel, subfamily J, member 9 |
| 1613955 | 3.45E−02 | Homo sapiens, clone MGC: 20633 IMAGE: 4761663, mRNA, complete cds |
| 165921 | 1.80E−02 | CEP2\|centrosomal protein 2 |
| 810120 | 3.97E−02 | LOC51160\|VPS28 protein |
| 814266 | 4.89E−02 | PRKCZ\|protein kinase C, zeta |
| 810124 | 8.98E−03 | PAFAH1B3\|platelet-activating factor acetylhydrolase, isoform lb, gamma subunit (29 kD) |
| 244307 | 1.69E−02 | SERPINE1\|serine (or cysteine) proteinase inhibitor, clade E (nexin, plasminogen activator inhibitor type 1), |
| | | member 1 |
| 951216 | 2.18E−02 | NDUFB10\|NADH dehydrogenase (ubiquinone) 1 beta subcomplex, 10 (22 kD, PDSW) |
| 2062825 | 7.66E−03 | KIAA0964\|KIAA0964 protein |
| 306575 | 2.86E−02 | DIPA\|hepatitis delta antigen-interacting protein A |
| 878652 | 9.48E−03 | PCOLCE\|procollagen C-endopeptidase enhancer |
| 1631746 | 5.77E−03 | POLM\|polymerase (DNA directed), mu |
| 23903 | 1.67E−02 | Homo sapiens clone 23903 mRNA sequence |
| 743114 | 2.28E−02 | HSPBP1\|hsp70-interacting protein |
| 123614 | 5.02E−03 | MGC4675\|hypothetical protein MGC4675 |
| 824108 | 4.41E−02 | SCAND1\|SCAN domain-containing 1 |
| 51097 | 3.22E−02 | BAIAP3\|BAI1-associated protein 3 |
| 770588 | 2.28E−02 | Homo sapiens TTF-I interacting peptide 20 mRNA, partial cds |
| 130835 | 4.52E−02 | Homo sapiens, Similar to clone FLB3816, clone IMAGE: 3454380, mRNA |
| 725407 | 4.97E−03 | SMURF1\|E3 ubiquitin ligase SMURF1 |
| 66952 | 1.07E−02 | ZNF205\|zinc finger protein 205 |
| 345487 | 4.70E−03 | Homo sapiens, clone MGC: 23280 IMAGE: 4637504, mRNA, complete cds |
| 1591264 | 4.54E−02 | TALDO1\|transaldolase 1 |
| 1868534 | 3.48E−02 | MGC2408\|hypothetical protein MGC2408 |
| 951080 | 3.24E−02 | RECQL4\|RecQ protein-like 4 |
| 144740 | 1.22E−02 | SDCCAG28\|serologically defined colon cancer antigen 28 |
| 625693 | 4.10E−02 | MGC10911\|hypothetical protein MGC10911 |
| 1563792 | 3.66E−02 | LOC51333\|mesenchymal stem cell protein DSC43 |
| 194214 | 4.39E−02 | TGIF\|TGFB-induced factor (TALE family homeobox) |
| 1845744 | 2.17E−03 | EST |
| 356992 | 3.82E−02 | HSPC023\|HSPC023 protein |
| 282428 | 3.71E−02 | Homo sapiens, Similar to RIKEN cDNA 9030409E16 gene, clone MGC: 26939 IMAGE: 4796761, mRNA, |
| | | complete cds |
| 254010 | 3.08E−02 | LOC51175\|epsilon-tubulin |
| 264646 | 3.76E−02 | HGS\|hepatocyte growth factor-regulated tyrosine kinase substrate |
| 724615 | 4.54E−02 | CHC1\|chromosome condensation 1 |
| 647767 | 2.91E−03 | MGC4758\|similar to RIKEN cDNA 2310040G17 gene |
| 951233 | 3.43E−02 | PSMB3\|proteasome (prosome, macropain) subunit, beta type, 3 |
| 814287 | 6.96E−04 | XRCC3\|X-ray repair complementing defective repair in Chinese hamster cells 3 |
| 2013094 | 1.18E−02 | KIF1C\|kinesin family member 1C |
| 366834 | 3.25E−02 | EVPL\|envoplakin |
| 51328 | 2.05E−02 | CDC34\|cell division cycle 34 |
| 842846 | 3.82E−02 | TIMP2\|tissue inhibitor of metalloproteinase 2 |
| 1640586 | 3.59E−02 | DUSP3\|dual specificity phosphatase 3 (vaccinia virus phosphatase VH1-related) |
| 740801 | 6.02E−03 | BCKDHA\|branched chain keto acid dehydrogenase E1, alpha polypeptide (maple syrup urine disease) |
| 68717 | 3.22E−02 | UCK1\|uridine-cytidine kinase 1 |
| 33478 | 4.62E−02 | FPGS\|folylpolyglutamate synthase |
| 813490 | 1.67E−02 | CORO1C\|coronin, actin-binding protein, 1C |
| 415136 | 7.38E−03 | ESTs, Weakly similar to T00370 hypothetical protein KIAA0659 [H. sapiens] |
| 725284 | 2.05E−03 | PHKG2\|phosphorylase kinase, gamma 2 (testis) |
| 1868626 | 5.84E−03 | PFKL\|phosphofructokinase, liver |
| 882488 | 4.21E−02 | TERF2\|telomeric repeat binding factor 2 |
| 785459 | 3.08E−02 | SMTN\|smoothelin |
| 813499 | 3.82E−02 | SSSCA1\|Sjogrens syndrome/scleroderma autoantigen 1 |
| 1473131 | 3.07E−02 | TLE2\|transducin-like enhancer of split 2, homolog of Drosophila E(sp1) |
| 632137 | 2.02E−02 | SIVA\|CD27-binding (Siva) protein |
| 784589 | 4.57E−02 | MMP15\|matrix metalloproteinase 15 (membrane-inserted) |
| 811897 | 4.55E−02 | MKL1\|megakaryoblastic leukemia (translocation) 1 |
| 1486099 | 4.00E−02 | TP73\|tumor protein p73 |
| 145491 | 1.14E−02 | PCDH1\|protocadherin 1 (cadherin-like 1) |
| 1946069 | 3.91E−04 | SPHK1\|sphingosine kinase 1 |
| 854079 | 3.55E−02 | ACTN1\|actinin, alpha 1 |
| 965223 | 2.83E−02 | TK1\|thymidine kinase 1, soluble |
| 824132 | 2.18E−02 | Homo sapiens, Similar to cofactor required for Sp1 transcriptional activation, subunit 8 (34 kD), clone MGC: 11274 |
| | | IMAGE: 3944264, mRNA, complete cds |
| 2108077 | 4.87E−03 | LOC51016\|CGI-112 protein |
| 22991 | 1.34E−02 | SUPT6H\|suppressor of Ty (S. cerevisiae) 6 homolog |
| 796968 | 2.31E−02 | KIAA1534\|KIAA1534 protein |
| 2326019 | 2.38E−02 | COX5B\|cytochrome c oxidase subunit Vb |
| 1637732 | 1.76E−02 | PPAN\|peter pan (Drosophila) homolog |
| 1580874 | 2.45E−03 | CORO2A\|coronin, actin-binding protein, 2A |
| 154466 | 1.80E−02 | STUB1\|STIP1 homology and U-Box containing protein 1 |
| 1474955 | 3.54E−02 | TAF15\|TAF15 RNA polymerase II, TATA box binding protein (TBP)-associated factor, 68 kD |
| 197727 | 2.95E−02 | PEMT\|phosphatidylethanolamine N-methyltransferase |
| 346604 | 1.76E−02 | AGER\|advanced glycosylation end product-specific receptor |
| 592818 | 6.44E−03 | KIAA1437\|hypothetical protein FLJ10337 |
| 2043418 | 3.39E−02 | CRF\|C1q-related factor |
| 842794 | 1.86E−02 | KIAA1668\|KIAA1668 protein |
| 1926769 | 3.16E−02 | SCNN1B\|sodium channel, nonvoltage-gated 1, beta (Liddle syndrome) |
| 882571 | 9.94E−03 | OAZIN\|ornithine decarboxylase antizyme inhibitor |
| 156211 | 8.98E−03 | ATP6B1\|ATPase, H+ transporting, lysosomal (vacuolar proton pump), beta polypeptide, 56/58 kD, isoform 1 (Renal |
| | | tubular acidosis with deafness) |
| 2307514 | 1.67E−02 | MLC1\|KIAA0027 protein |
| 154610 | 3.14E−03 | MGC3248\|dynactin 4 |
| 80708 | 2.51E−03 | UFD1L\|ubiquitin fusion degradation 1-like |
| 770910 | 3.28E−02 | ELF3\|E74-like factor 3 (ets domain transcription factor, epithelial-specific) |
| 753860 | 4.32E−02 | SLC25A13\|solute carrier family 25, member 13 (citrin) |
| 772377 | 3.45E−02 | Homo sapiens mRNA; cDNA DKFZp761H229 (from clone DKFZp761H229); partial cds |
| 34370 | 1.34E−02 | PLEC1\|plectin 1, intermediate filament binding protein, 500 kD |
| 271102 | 7.55E−03 | CCS\|copper chaperone for superoxide dismutase |
| 280934 | 1.77E−02 | MVD\|mevalonate (diphospho) decarboxylase |
| 140574 | 2.08E−02 | SCYD1\|small inducible cytokine subfamily D (Cys-X3-Cys), member 1 (fractalkine, neurotactin) |
| 1575410 | 1.51E−03 | Homo sapiens, Similar to RIKEN cDNA 2700064H14 gene, clone MGC: 21390 IMAGE: 4519078, mRNA, |
| | | complete cds |
| 1509761 | 2.06E−03 | KRTHB6\|keratin, hair, basic, 6 (monilethrix) |
| 68818 | 2.97E−03 | Homo sapiens, clone IMAGE: 3957135, mRNA, partial cds |
| 813807 | 7.03E−03 | RNF25\|ring finger protein 25 |
| 432075 | 1.05E−03 | TSSC4\|tumor suppressing subtransferable candidate 4 |
| 813738 | 3.20E−03 | BRF1\|BRF1 homolog, subunit of RNA polymerase III transcription initiation factor IIIB (S. cerevisiae) |
| 857652 | 1.93E−02 | PPT2\|palmitoyl-protein thioesterase 2 |
| 898237 | 3.61E−02 | BAT3\|HLA-B associated transcript 3 |
| 770856 | 2.69E−02 | DKFZP564D0478\|hypothetical protein DKFZp564D0478 |
| 760224 | 1.68E−03 | XRCC1\|X-ray repair complementing defective repair in Chinese hamster cells 1 |
| 85804 | 2.70E−02 | FLJ21918\|hypothetical protein FLJ21918 |
| 1607741 | 2.44E−02 | FLJ10385\|hypothetical protein FLJ10385 |
| 512410 | 2.91E−04 | RNASEHI\|ribonuclease HI, large subunit |
| 2326112 | 2.98E−02 | RPL22\|ribosomal protein L22 |
| 32927 | 1.89E−02 | FBXL6\|f-box and leucine-rich repeat protein 6 |
| 744047 | 2.47E−03 | PLK\|polo (Drosophia)-like kinase |
| 785707 | 3.67E−02 | PRC1\|protein regulator of cytokinesis 1 |
| 471200 | 1.14E−02 | LOC51042\|zinc finger protein |
| 263894 | 3.56E−02 | QPRT\|quinolinate phosphoribosyltransferase (nicotinate-nucleotide pyrophosphorylase (carboxylating)) |

### Example III

### Molecular Signature that Correlates with Recurrence of Breast Cancer

A molecular signature that correlates with recurrence of breast cancer after removal of cancer by surgery was identified as follows. Breast cancer tissue removed by surgery was microdissected (“laser captured”) to isolate breast cancer cells. The expression levels of multiple genes in the cells were used to identify those that correlate with cancer recurrence. The set of genes that correlate was identified by using a cox proportional hazard regression model using a single gene at a time as a covariate. Genes were selected with p<0.01 derived from the regression model. 396 genes were selected that correlated with recurrence, and they are listed in Table 4. The sign of the coefficient values in Table 4 correspond to whether a gene is positively or negatively correlated with survival outcome. A positive coefficient means that the gene is positively correlated (overexpressed) in patients with a poor (shorter) survival outcome and negatively correlated (underexpressed) in patients with a good or better (longer) survival outcome. A negative coefficient means that the gene is positively correlated (overexpressed) in patients with a good or better (longer) survival outcome and negatively correlated (underexpressed) in patients with a poor (shorter) survival outcome.

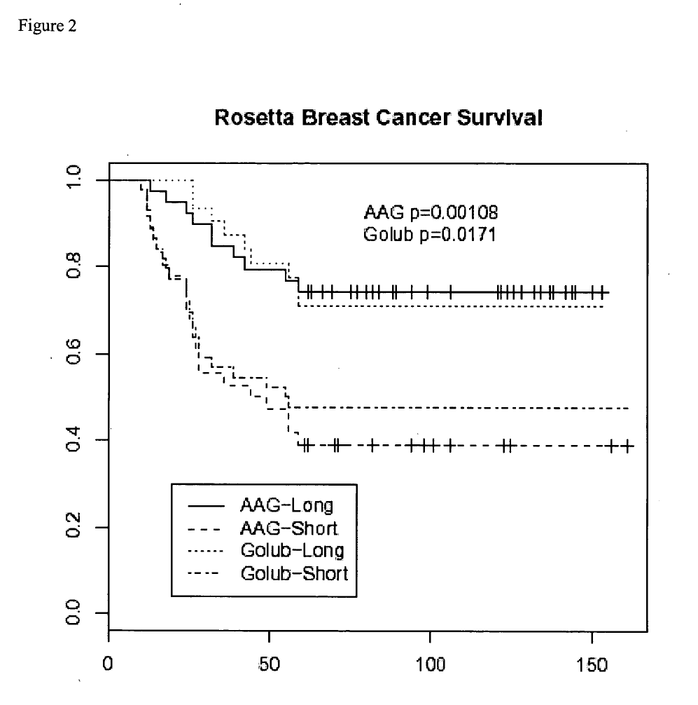

To validate this signature, an independent dataset of gene expression (van't Veer et al., supra) with clinical outcome (survival) was challenged with this signature. Of the 396 genes in Table 4, 297 genes overlapped with those examined in by van't Veer et al. and were thus used to determine whether this 297 gene set was correlative to overall survival. The 297 gene signature (identities of the genes are presented in Table 5 via their Clone ID, GenBank ID, and Unigene ID numbers) segregates the survival data (patient population) of van't Veer et al. into “long” and “short” groups with significantly different overall survival curves as shown by the lines identified as “AAG-Long” and “AAG-Short” in FIG. 2. Like FIG. 1, the horizontal axis of, FIG. 2 is in months and the vertical axis is in survival probability (where 1.0 is survival of 100% of the subjects in a group and 0.5 is survival of 50% of the subjects in a group). The line identified as “AAG-Short” is the lowest line at time points of about 60 months and higher.

FIG. 2 also shows the comparison of this 297 gene set with that of a set of 17 genes correlated with matastasis described by Ramaswamy et al. (supra, see Table 1 therein). The curves corresponding to the Ramaswamy et al. signature are identified as “Golub-Long” and “Golub-Short”. FIG. 2 shows that 297 gene signature separated the survival curves to a greater extent than the 17 gene set of Ramaswamy et al. The 297 gene signature also correlated with the data with a p value of 0.00106, which is approximately 10 fold better than the p value of 0.0171 for the Ramaswamy et al. 17 gene set.

TABLE 4
Genes, the expressions of which correlate with the breast cancer recurrence

| CloneID | p value | coef | description |
|---|---|---|---|
| 229901 | 9.71E−07 | −1.95 | CTSO\|cathepsin O |
| 1635618 | 1.71E−06 | 2.07 | KIAA1115\|KIAA1115 protein |
| 142022 | 3.98E−06 | −1.62 | ESTs |
| 774446 | 5.70E−06 | 0.79 | ADM\|adrenomedullin |
| 85409 | 6.76E−06 | −1.46 | CREG\|cellular repressor of E1A-stimulated genes |
| 666169 | 9.91E−06 | −2.43 | MTR\|5-methyltetrahydrofolate-homocysteine methyltransferase |
| 2015148 | 1.95E−05 | 1.16 | GIT1\|G protein-coupled receptor kinase-interactor 1 |
| 628357 | 2.02E−05 | 1.95 | ACTN3\|actinin, alpha 3 |
| 815235 | 3.12E−05 | 2.10 | RCD-8\|autoantigen |
| 491053 | 4.46E−05 | −3.50 | ARIH2\|ariadne (Drosophila) homolog 2 |
| 823819 | 5.35E−05 | −1.73 | |
| 487297 | 5.49E−05 | −1.60 | CAP2\|adenylyl cyclase-associated protein 2 |
| 782385-2 | 5.53E−05 | −2.08 | DKFZP566D193\|DKFZP566D193 protein |
| 26811 | 8.32E−05 | −1.99 | XRCC4\|X-ray repair complementing defective repair in Chinese hamster cells 4 |
| 341316 | 8.81E−05 | −1.38 | HTATSF1\|HIV TAT specific factor 1 |
| 743182 | 1.01E−04 | 1.22 | DJ37E16.5\|hypothetical protein dJ37E16.5 |
| 310584 | 1.09E−04 | −2.25 | ARL1\|ADP-ribosylation factor-like 1 |
| 2016426 | 1.22E−04 | 2.79 | KIAA0664\|KIAA0664 protein |
| 502891 | 1.22E−04 | −1.46 | FLJ11184\|hypothetical protein FLJ11184 |
| 202577 | 1.30E−04 | −0.87 | HNMT\|histamine N-methyltransferase |
| 1637282 | 1.31E−04 | 1.23 | HK2\|hexokinase 2 |
| 150003 | 1.40E−04 | −0.99 | FLJ13187\|phafin 2 |
| 366209 | 1.41E−04 | −1.10 | ESTs |
| 810063 | 1.99E−04 | −1.45 | GFER\|growth factor, erv1 (S. cerevisiae)-like (augmenter of liver regeneration) |
| 855800 | 2.29E−04 | −1.18 | PREP\|prolyl endopeptidase |
| 781222 | 2.56E−04 | 1.48 | TIAF1\|TGFB1-induced anti-apoptotic factor 1 |
| 897164 | 2.72E−04 | −0.95 | CTNNA1\|catenin (cadherin-associated protein), alpha 1 (102 kD) |
| 134270 | 2.87E−04 | −1.19 | Human hbc647 mRNA sequence |
| 745360 | 2.91E−04 | −1.14 | HAT1\|histone acetyltransferase 1 |
| 2313673 | 2.91E−04 | 1.59 | LOC50999\|CGI-100 protein |
| 309469 | 2.98E−04 | 1.38 | KIAA1725\|KIAA1725 protein |
| 2018808 | 3.28E−04 | −1.08 | PRCP\|prolylcarboxypeptidase (angiotensinase C) |
| 108425-2 | 3.29E−04 | −1.70 | ESTs, Weakly similar to JC5314 CDC28/cdc2-like kinase associating arginine-serine |
| | | | cyclophilin [H. sapiens] |
| 788745 | 3.30E−04 | −1.72 | WS-3\|novel RGD-containing protein |
| 1638827 | 3.49E−04 | 1.19 | RFPL3S\|ret finger protein-like 3 antisense |
| 1670688 | 3.59E−04 | −1.89 | BACH2\|BTB and CNC homology 1, basic leucine zipper transcription factor 2 |
| 75886 | 3.95E−04 | −1.08 | ESTs, Weakly similar to E54024 protein kinase [H. sapiens] |
| 85614 | 4.01E−04 | −1.40 | LEPROTL1\|leptin receptor overlapping transcript-like 1 |
| 1737724 | 4.12E−04 | 1.55 | LRRN1\|leucine-rich repeat protein, neuronal 1 |
| 155920 | 4.23E−04 | 1.95 | FLJ10211\|hypothetical protein FLJ10211 |
| 306933 | 4.24E−04 | 1.27 | Homo sapiens clone 25012 mRNA sequence |
| 1732033 | 4.27E−04 | −1.94 | FLJ14427\|hypothetical protein FLJ14427 |
| 815167 | 4.37E−04 | −1.54 | PLEKHA3\|pleckstrin homology domain-containing, family A (phosphoinositide binding specific) member 3 |
| 166199 | 4.51E−04 | 1.87 | ADRBK1\|adrenergic, beta, receptor kinase 1 |
| 50794 | 4.58E−04 | 0.74 | ZNF133\|zinc finger protein 133 (clone pHZ-13) |
| 504201 | 4.68E−04 | 1.49 | Homo sapiens, clone IMAGE: 3677194, mRNA, partial cds |
| 1609748 | 4.92E−04 | −0.82 | MGC10882\|hypothetical protein MGC10882 |
| 773375 | 5.23E−04 | −1.23 | |
| 40173 | 5.66E−04 | 1.42 | MAST205\|KIAA0807 protein |
| 1416782 | 5.66E−04 | 0.63 | CKB\|creatine kinase, brain |
| 826286 | 5.82E−04 | 1.86 | IMP13\|importin 13 |
| 235056 | 5.94E−04 | 1.06 | 24432\|hypothetical protein 24432 |
| 824510 | 6.13E−04 | 1.26 | LOC51647\|CGI-128 protein |
| 796255 | 6.27E−04 | −1.13 | MRPS14\|mitochondrial ribosomal protein S14 |
| 785459 | 6.38E−04 | 0.92 | SMTN\|smoothelin |
| 39677 | 6.40E−04 | −2.30 | FLJ10702\|hypothetical protein FLJ10702 |
| 149539 | 6.67E−04 | −1.21 | MKP-7\|MAPK phosphatase-7 |
| 32231 | 7.03E−04 | 0.91 | FLJ12442\|hypothetical protein FLJ12442 |
| 1466237 | 7.16E−04 | 1.54 | TES\|testis derived transcript (3 LIM domains) |
| 155050 | 7.39E−04 | −1.42 | MDS025\|hypothetical protein MDS025 |
| 84287 | 7.42E−04 | 1.47 | ESTs |
| 845513 | 7.46E−04 | 1.34 | AP47\|clathrin-associated protein AP47 |
| 1903067 | 7.48E−04 | 2.66 | C21orf18\|chromosome 21 open reading frame 18 |
| 83653 | 7.55E−04 | −2.30 | HSPC128\|HSPC128 protein |
| 1603583 | 7.80E−04 | −0.81 | SH3BGRL\|SH3 domain binding glutamic acid-rich protein like |
| 744047 | 8.09E−04 | 0.94 | PLK\|polo (Drosophia)-like kinase |
| 1947381 | 8.56E−04 | 1.05 | FLJ22329\|hypothetical protein FLJ22329 |
| 884677 | 8.60E−04 | −1.47 | Homo sapiens, clone IMAGE: 3611719, mRNA, partial cds |
| 84068 | 8.93E−04 | −1.52 | CL25084\|hypothetical protein |
| 529147 | 9.17E−04 | −1.20 | VPS26\|vacuolar protein sorting 26 (yeast homolog) |
| 1693357 | 9.35E−04 | 0.99 | EDN2\|endothelin 2 |
| 26856 | 9.51E−04 | 0.96 | FLOT2\|flotillin 2 |
| 767753 | 9.62E−04 | −1.49 | RFX5\|regulatory factor X, 5 (influences HLA class II expression) |
| 2322079 | 1.01E−03 | 1.02 | |
| 815057 | 1.03E−03 | −1.11 | FLJ10652\|hypothetical protein FLJ10652 |
| 2062453 | 1.05E−03 | 0.74 | DKFZP727G051\|DKFZP727G051 protein |
| 126221 | 1.06E−03 | 1.15 | TPD52L2\|tumor protein D52-like 2 |
| 290536 | 1.07E−03 | 1.39 | ESTs, Weakly similar to T43483 translation initiation factor IF-2 homolog [H. sapiens] |
| 505299 | 1.12E−03 | −2.27 | BBP\|beta-amyloid binding protein precursor |
| 796694 | 1.12E−03 | 2.00 | BIRC5\|baculoviral IAP repeat-containing 5 (survivin) |
| 786053 | 1.13E−03 | 1.27 | Homo sapiens cDNA FLJ30898 fis, clone FEBRA2005572 |
| 145136 | 1.14E−03 | −1.48 | Homo sapiens cDNA FLJ13103 fis, clone NT2RP3002304 |
| 140951 | 1.17E−03 | 1.06 | ACTN4\|actinin, alpha 4 |
| 725395 | 1.18E−03 | −1.14 | UBE2L6\|ubiquitin-conjugating enzyme E2L 6 |
| 295781 | 1.20E−03 | −0.86 | MGC9084\|hypothetical protein MGC9084 |
| 267590 | 1.20E−03 | 1.37 | KIAA0330\|calcineurin binding protein 1 |
| 299388 | 1.21E−03 | 1.48 | PP15\|nuclear transport factor 2 (placental protein 15) |
| 1506046 | 1.24E−03 | 1.00 | FLJ10815\|hypothetical protein FLJ10815 |
| 250313 | 1.25E−03 | −1.57 | ESTs |
| 1882051 | 1.27E−03 | −1.58 | FLJ20080\|hypothetical protein FLJ20080 |
| 898312 | 1.27E−03 | 1.08 | TRAF4\|TNF receptor-associated factor 4 |
| 712482 | 1.31E−03 | −1.73 | APTX\|aprataxin |
| 1926249 | 1.31E−03 | 1.28 | LOC58509\|NY-REN-24 antigen |
| 26507 | 1.34E−03 | 1.54 | |
| 758318 | 1.38E−03 | −1.32 | FBXO3\|F-box only protein 3 |
| 785708 | 1.42E−03 | −1.51 | ESTs, Weakly similar to O4HUD1 debrisoquine 4-hydroxylase [H. sapiens] |
| 842968 | 1.42E−03 | 1.38 | BUB1B\|budding uninhibited by benzimidazoles 1 (yeast homolog), beta |
| 34778-2 | 1.45E−03 | 0.87 | VEGF\|vascular endothelial growth factor |
| 742007 | 1.45E−03 | −1.42 | KIAA0146\|KIAA0146 protein |
| 1030351 | 1.48E−03 | −1.50 | SCYB11\|small inducible cytokine subfamily B (Cys-X-Cys), member 11 |
| 741474 | 1.54E−03 | 0.79 | GPI\|glucose phosphate isomerase |
| 827171 | 1.61E−03 | −0.90 | LRRC2\|leucine-rich repeat-containing 2 |
| 266747 | 1.61E−03 | −0.97 | Homo sapiens, Similar to RIKEN cDNA 2010001O09 gene, clone MGC: 21387 IMAGE: 4471592, mRNA, |
| | | | complete cds |
| 52103 | 1.62E−03 | −1.49 | FLJ23045\|hypothetical protein FLJ23045 |
| 795893 | 1.63E−03 | 1.91 | PPP1R15A\|protein phosphatase 1, regulatory (inhibitor) subunit 15A |
| 782689 | 1.64E−03 | 0.68 | SLC6A8\|solute carrier family 6 (neurotransmitter transporter, creatine), member 8 |
| 724615 | 1.66E−03 | 1.12 | CHC1\|chromosome condensation 1 |
| 138788 | 1.68E−03 | −0.87 | PRLR\|prolactin receptor |
| 815535 | 1.68E−03 | 1.37 | TCOF1\|Treacher Collins-Franceschetti syndrome 1 |
| 261481 | 1.70E−03 | −1.08 | CUL3\|cullin 3 |
| 1475738 | 1.72E−03 | −1.99 | RPS25\|ribosomal protein S25 |
| 70606 | 1.76E−03 | −0.92 | ESTs |
| 345423 | 1.80E−03 | −1.57 | DKFZP564M112\|likely ortholog of preimplantation protein 3 |
| 414992 | 1.84E−03 | 0.90 | LOC57106\|K562 cell-derived leucine-zipper-like protein 1 |
| 770588 | 1.85E−03 | 1.41 | Homo sapiens TTF-I interacting peptide 20 mRNA, partial cds |
| 163558 | 1.86E−03 | 1.91 | SIRT6\|sirtuin (silent mating type information regulation 2, S. cerevisiae, homolog) 6 |
| 840865 | 1.92E−03 | 1.66 | MACS\|myristoylated alanine-rich protein kinase C substrate (MARCKS, 80K-L) |
| 23831 | 1.92E−03 | 0.51 | ALDOC\|aldolase C, fructose-bisphosphate |
| 23772 | 1.95E−03 | 1.24 | LZTR1\|leucine-zipper-like transcriptional regulator, 1 |
| 756662 | 1.95E−03 | 1.40 | KIAA0943\|KIAA0943 protein |
| 784150 | 1.97E−03 | −1.24 | RAB31\|RAB31, member RAS oncogene family |
| 242706 | 1.99E−03 | −1.48 | HSPC274\|HSPC274 protein |
| 1947804 | 2.04E−03 | 1.13 | TREX1\|three prime repair exonuclease 1 |
| 279085 | 2.07E−03 | 1.19 | MYO9B\|myosin IXB |
| 109316 | 2.08E−03 | −1.17 | SERPINA3\|serine (or cysteine) proteinase inhibitor, clade A (alpha-1 antiproteinase, antitrypsin), member 3 |
| 840506 | 2.08E−03 | −1.53 | 3-Apr\|apoptosis related protein APR-3 |
| 491486 | 2.09E−03 | −1.24 | LOC51578\|adrenal gland protein AD-004 |
| 1734309 | 2.13E−03 | 0.75 | SPAG4\|sperm associated antigen 4 |
| 810983 | 2.16E−03 | 1.41 | DKFZP434H132\|DKFZP434H132 protein |
| 47795 | 2.16E−03 | −1.31 | ZNF161\|zinc finger protein 161 |
| 307933 | 2.17E−03 | −2.26 | NDUFB5\|NADH dehydrogenase (ubiquinone) 1 beta subcomplex, 5 (16 kD, SGDH) |
| 897971 | 2.18E−03 | −2.23 | COPB\|coatomer protein complex, subunit beta |
| 743810 | 2.20E−03 | 2.32 | MGC2577\|hypothetical protein MGC2577 |
| 860000 | 2.21E−03 | 1.61 | RFC2\|replication factor C (activator 1) 2 (40 kD) |
| 262739 | 2.23E−03 | −0.97 | P125\|Sec23-interacting protein p125 |
| 754537 | 2.32E−03 | −0.79 | Homo sapiens cDNA FLJ10229 fis, clone HEMBB1000136 |
| 37708 | 2.32E−03 | 0.79 | MGC3101\|hypothetical protein MGC3101 |
| 1752548 | 2.32E−03 | −2.59 | CNGB3\|cyclic nucleotide gated channel beta 3 |
| 307740 | 2.37E−03 | −1.12 | ESTs |
| 51063 | 2.43E−03 | 0.86 | ESTs |
| 277999 | 2.47E−03 | −1.16 | DKFZP434D193\|DKFZP434D193 protein |
| 768452 | 2.47E−03 | −0.94 | Homo sapiens EST from clone 491476, full insert |
| 856164 | 2.48E−03 | 1.26 | AS3\|androgen-induced prostate proliferative shutoff associated protein |
| 2009779 | 2.48E−03 | −1.24 | RAB5EP\|rabaptin-5 |
| 755578 | 2.48E−03 | 0.61 | SLC7A5\|solute carrier family 7 (cationic amino acid transporter, y+ system), member 5 |
| 1913943 | 2.52E−03 | 0.78 | ESTs, Weakly similar to I38022 hypothetical protein [H. sapiens] |
| 767068 | 2.53E−03 | 0.54 | DKFZP586G1517\|DKFZP586G1517 protein |
| 739191 | 2.54E−03 | 1.74 | ZNF261\|zinc finger protein 261 |
| 786674 | 2.59E−03 | 0.51 | SOX2\|SRY (sex determining region Y)-box 2 |
| 795936 | 2.60E−03 | −1.62 | TSN\|translin |
| 687289 | 2.64E−03 | −2.20 | Homo sapiens, clone MGC: 3245 IMAGE: 3505639, mRNA, complete cds |
| 685516 | 2.67E−03 | −0.59 | GPCR150\|putative G protein-coupled receptor |
| 38244 | 2.70E−03 | 1.22 | FLJ12587\|hypothetical protein FLJ12587 |
| 855872 | 2.70E−03 | 1.62 | NRD1\|nardilysin (N-arginine dibasic convertase) |
| 2125819 | 2.70E−03 | 1.22 | BAX\|BCL2-associated X protein |
| 2307119 | 2.74E−03 | 1.03 | INPP4A\|inositol polyphosphate-4-phosphatase, type I, 107 kD |
| 2449343 | 2.74E−03 | 0.71 | PTPRH\|protein tyrosine phosphatase, receptor type, H |
| 325515 | 2.85E−03 | −0.73 | FLJ10980\|hypothetical protein FLJ10980 |
| 824132 | 2.87E−03 | 1.22 | Homo sapiens, Similar to cofactor required for Sp1 transcriptional activation, subunit 8 (34 kD), clone |
| | | | MGC: 11274 IMAGE: 3944264, mRNA, complete cds |
| 1500241 | 2.88E−03 | −0.51 | C1orf24\|chromosome 1 open reading frame 24 |
| 811790 | 2.89E−03 | −1.19 | DKFZP564G0222\|DKFZP564G0222 protein |
| 770835 | 2.94E−03 | −1.07 | BCKDHB\|branched chain keto acid dehydrogenase E1, beta polypeptide (maple syrup urine disease) |
| 796114 | 2.94E−03 | −1.18 | SIRT1\|sirtuin (silent mating type information regulation 2, S. cerevisiae, homolog) 1 |
| 884438 | 2.96E−03 | −1.18 | NFE2L2\|nuclear factor (erythroid-derived 2)-like 2 |
| 150897 | 3.00E−03 | 0.50 | B3GNT3\|UDP-GlcNAc: betaGal beta-1,3-N-acetylglucosaminyltransferase 3 |
| 1519013 | 3.04E−03 | 0.95 | Homo sapiens, clone IMAGE: 3537447, mRNA, partial cds |
| 323693 | 3.04E−03 | 1.25 | AP1S1\|adaptor-related protein complex 1, sigma 1 subunit |
| 124046 | 3.09E−03 | 1.30 | JAZ\|double-stranded RNA-binding zinc finger protein JAZ |
| 843091 | 3.10E−03 | 0.88 | MGC20533\|similar to RIKEN cDNA 2410004L22 gene (M. musculus) |
| 165828 | 3.10E−03 | 0.75 | FHOS\|FH1/FH2 domain-containing protein |
| 159535 | 3.14E−03 | −1.22 | ESTs |
| 826256 | 3.18E−03 | −0.68 | TM7SF1\|transmembrane 7 superfamily member 1 (upregulated in kidney) |
| 68345 | 3.21E−03 | 1.43 | ITPR3\|inositol 1,4,5-triphosphate receptor, type 3 |
| 128426 | 3.27E−03 | 0.63 | WBSCR14\|Williams-Beuren syndrome chromosome region 14 |
| 1601601 | 3.28E−03 | 1.73 | CSF2\|colony stimulating factor 2 (granulocyte-macrophage) |
| 1474164 | 3.36E−03 | 1.51 | FLJ12886\|hypothetical protein FLJ12886 |
| 1871423 | 3.39E−03 | −1.27 | CDC23\|CDC23 (cell division cycle 23, yeast, homolog) |
| 1908840 | 3.45E−03 | −1.58 | ZNF174\|zinc finger protein 174 |
| 68557 | 3.45E−03 | 1.50 | FABP1\|fatty acid binding protein 1, liver |
| 769712 | 3.46E−03 | 1.64 | GAK\|cyclin G associated kinase |
| 767477 | 3.47E−03 | −0.91 | ANKRA2\|ankyrin repeat, family A (RFXANK-like), 2 |
| 41647 | 3.49E−03 | −0.66 | PTPRT\|protein tyrosine phosphatase, receptor type, T |
| 767495 | 3.50E−03 | −0.51 | GLI3\|GLI-Kruppel family member GLI3 (Greig cephalopolysyndactyly syndrome) |
| 754582 | 3.50E−03 | −1.05 | EVI2A\|ecotropic viral integration site 2A |
| 166268 | 3.59E−03 | 1.61 | SR-A1\|serine arginine-rich pre-mRNA splicing factor SR-A1 |
| 769004 | 3.61E−03 | −2.39 | MPHOSPH1\|M-phase phosphoprotein 1 |
| 280249 | 3.66E−03 | 1.37 | KLF7\|Kruppel-like factor 7 (ubiquitous) |
| 198874 | 3.67E−03 | 1.33 | FLJ10922\|hypothetical protein FLJ10922 |
| 74738 | 3.74E−03 | 0.94 | MGC20486\|hypothetical protein MGC20486 |
| 130153 | 3.75E−03 | 1.15 | SUPT5H\|suppressor of Ty (S. cerevisiae) 5 homolog |
| 51469 | 3.82E−03 | 1.17 | ADPRTL2\|ADP-ribosyltransferase (NAD+; poly(ADP-ribose) polymerase)-like 2 |
| 122739 | 3.82E−03 | 1.28 | FLJ21918\|hypothetical protein FLJ21918 |
| 782787 | 3.83E−03 | −0.98 | FLJ21347\|hypothetical protein FLJ21347 |
| 1894519 | 3.84E−03 | −1.35 | FLJ12085\|hypothetical protein FLJ12085 |
| 244307 | 3.87E−03 | 0.92 | SERPINE1\|serine (or cysteine) proteinase inhibitor, clade E (nexin, plasminogen activator inhibitor type 1), |
| | | | member 1 |
| 137836 | 3.92E−03 | −0.99 | PDCD10\|programmed cell death 10 |
| 1702742 | 3.95E−03 | 0.63 | SLC7A5\|solute carrier family 7 (cationic amino acid transporter, y+ system), member 5 |
| 813490 | 4.00E−03 | 0.99 | CORO1C\|coronin, actin-binding protein, 1C |
| 770518 | 4.01E−03 | 0.99 | KIAA0618\|KIAA0618 gene product |
| 825176 | 4.02E−03 | −1.00 | FLJ11273\|hypothetical protein FLJ11273 |
| 530954 | 4.07E−03 | 1.17 | CFL2\|cofilin 2 (muscle) |
| 1588973 | 4.08E−03 | −1.35 | IMAGE3451454\|hypothetical protein IMAGE3451454 |
| 769537 | 4.13E−03 | 0.94 | ECH1\|enoyl Coenzyme A hydratase 1, peroxisomal |
| 490753 | 4.15E−03 | 1.22 | FLJ20420\|hypothetical protein FLJ20420 |
| 488505 | 4.16E−03 | 0.73 | SLC6A8\|solute carrier family 6 (neurotransmitter transporter, creatine), member 8 |
| 209066-2 | 4.18E−03 | 0.68 | STK15\|serine/threonine kinase 15 |
| 767236 | 4.30E−03 | −1.07 | CGI-51\|CGI-51 protein |
| 503096 | 4.31E−03 | 1.10 | ESTs |
| 1575410 | 4.33E−03 | 1.14 | Homo sapiens, Similar to RIKEN cDNA 2700064H14 gene, clone MGC: 21390 IMAGE: 4519078, mRNA, |
| | | | complete cds |
| 745437 | 4.33E−03 | −1.55 | ESTs |
| 590338 | 4.33E−03 | −0.86 | LOC51065\|40S ribosomal protein S27 isoform |
| 757328 | 4.34E−03 | 1.43 | FLJ22678\|hypothetical protein FLJ22678 |
| 726786 | 4.35E−03 | −1.69 | MGC2821\|hypothetical protein MGC2821 |
| 51010 | 4.35E−03 | 1.13 | FLJ20859\|hypothetical protein FLJ20859 |
| 770430 | 4.40E−03 | 1.26 | DKFZP434D0421\|hypothetical protein DKFZp434D0421 |
| 365919 | 4.40E−03 | −1.03 | STAU\|staufen (Drosophila, RNA-binding protein) |
| 44443 | 4.40E−03 | −1.08 | SCYE1\|small inducible cytokine subfamily E, member 1 (endothelial monocyte-activating) |
| 811907 | 4.50E−03 | 0.96 | FLJ22056\|hypothetical protein FLJ22056 |
| 502151 | 4.52E−03 | 0.56 | SLC16A3\|solute carrier family 16 (monocarboxylic acid transporters), member 3 |
| 950667 | 4.53E−03 | −1.02 | HRASLS\|HRAS-like suppressor |
| 742707 | 4.76E−03 | 1.33 | ESTs, Weakly similar to MUC2_HUMAN MUCIN 2 PRECURSOR [H. sapiens] |
| 299274 | 4.79E−03 | −0.71 | Homo sapiens cDNA FLJ32430 fis, clone SKMUS2001129, weakly similar to NAD-DEPENDENT |
| | | | METHANOL DEHYDROGENASE (EC 1.1.1.244) |
| 135303 | 4.79E−03 | −0.87 | HT007\|uncharacterized hypothalamus protein HT007 |
| 788511 | 4.80E−03 | 1.16 | RPS6KA1\|ribosomal protein S6 kinase, 90 kD, polypeptide 1 |
| 2062825 | 4.82E−03 | 0.77 | KIAA0964\|KIAA0964 protein |
| 686552 | 4.83E−03 | −1.23 | GOLPH1\|golgi phosphoprotein 1 |
| 586650 | 4.85E−03 | 1.05 | SLC29A1\|solute carrier family 29 (nucleoside transporters), member 1 |
| 2239290 | 4.86E−03 | −0.95 | SDF1\|stromal cell-derived factor 1 |
| 2502722 | 4.87E−03 | −0.60 | LOH11CR2A\|loss of heterozygosity, 11, chromosomal region 2, gene A |
| 587847 | 4.88E−03 | 0.81 | GPX2\|glutathione peroxidase 2 (gastrointestinal) |
| 2054896 | 4.89E−03 | −0.94 | FLJ21669\|hypothetical protein FLJ21669 |
| 812153 | 4.94E−03 | −1.14 | FLJ13081\|hypothetical protein FLJ13081 |
| 811888 | 4.97E−03 | −1.22 | DKFZP586F1122\|hypothetical protein DKFZp586F1122 similar to axotrophin |
| 504826 | 4.97E−03 | −1.31 | TFAM\|transcription factor A, mitochondrial |
| 1635695 | 5.01E−03 | 0.55 | GGA2\|Golgi-associated, gamma-adaptin ear containing, ARF-binding protein 2 |
| 1636166 | 5.07E−03 | 0.98 | KIAA0668\|KIAA0668 protein |
| 322511 | 5.09E−03 | −0.97 | Homo sapiens mRNA; cDNA DKFZp564D1462 (from clone DKFZp564D1462) |
| 26314 | 5.12E−03 | −1.13 | STXBP3\|syntaxin binding protein 3 |
| 2430676 | 5.16E−03 | 1.40 | EZFIT\|endothelial zinc finger protein induced by tumor necrosis factor alpha |
| 346545 | 5.19E−03 | 0.93 | Homo sapiens cDNA FLJ30346 fis, clone BRACE2007527 |
| 1592530 | 5.22E−03 | 0.94 | IP6K2\|mammalian inositol hexakisphosphate kinase 2 |
| 32684 | 5.25E−03 | −1.15 | RPL32\|ribosomal protein L32 |
| 279800 | 5.28E−03 | −1.19 | SLMAP\|sarcolemma associated protein |
| 1733935 | 5.30E−03 | 1.34 | DDX8\|DEAD/H (Asp-Glu-Ala-Asp/His) box polypeptide 8 (RNA helicase) |
| 824487 | 5.30E−03 | 1.09 | MGC2594\|hypothetical protein MGC2594 |
| 813281 | 5.35E−03 | −0.72 | WWP1\|WW domain-containing protein 1 |
| 150137 | 5.38E−03 | −1.29 | DKFZP564O123\|DKFZP564O123 protein |
| 135503 | 5.38E−03 | 1.38 | BRD4\|bromodomain-containing 4 |
| 780947 | 5.39E−03 | 0.92 | POLD1\|polymerase (DNA directed), delta 1, catalytic subunit (125 kD) |
| 884455 | 5.57E−03 | 1.04 | PRDX5\|peroxiredoxin 5 |
| 266500 | 5.63E−03 | −0.53 | ESTs |
| 51328 | 5.68E−03 | 1.00 | CDC34\|cell division cycle 34 |
| 897767 | 5.69E−03 | 2.04 | U5-100K\|prp28, U5 snRNP 100 kd protein |
| 811029 | 5.74E−03 | 0.89 | KIAA0365\|KIAA0365 gene product |
| 810391 | 5.74E−03 | 0.81 | HYAL1\|hyaluronoglucosaminidase 1 |
| 2306919 | 5.76E−03 | −0.93 | SLC35A3\|solute carrier family 35 (UDP-N-acetylglucosamine (UDP-GlcNAc) transporter), member 3 |
| 2018820 | 5.80E−03 | −1.19 | LRP3\|low density lipoprotein receptor-related protein 3 |
| 462939 | 5.82E−03 | −1.08 | Homo sapiens cDNA FLJ31303 fis, clone LIVER1000082 |
| 882488 | 5.85E−03 | 1.27 | TERF2\|telomeric repeat binding factor 2 |
| 262916 | 5.87E−03 | −1.27 | PPM1B\|protein phosphatase 1B (formerly 2C), magnesium-dependent, beta isoform |
| 1926575 | 5.90E−03 | −1.33 | CDX2\|caudal type homeo box transcription factor 2 |
| 814285 | 5.90E−03 | −1.34 | FLJ11240\|hypothetical protein FLJ11240 |
| 296190 | 5.92E−03 | −1.48 | KIAA0321\|KIAA0321 protein |
| 34852 | 5.93E−03 | −1.01 | BIRC2\|baculoviral IAP repeat-containing 2 |
| 1404396 | 5.95E−03 | 1.10 | PLCB3\|phospholipase C, beta 3 (phosphatidylinositol-specific) |
| 431869 | 6.00E−03 | 0.88 | Homo sapiens, clone IMAGE: 3506202, mRNA, partial cds |
| 884388 | 6.05E−03 | 1.21 | FLJ21103\|hypothetical protein FLJ21103 |
| 2313921 | 6.14E−03 | −0.91 | NDUFB3\|NADH dehydrogenase (ubiquinone) 1 beta subcomplex, 3 (12 kD, B12) |
| 824352 | 6.14E−03 | −1.35 | RAD23B\|RAD23 (S. cerevisiae) homolog B |
| 321945 | 6.15E−03 | −1.25 | ESTs |
| 140574 | 6.20E−03 | 0.42 | SCYD1\|small inducible cytokine subfamily D (Cys-X3-Cys), member 1 (fractalkine, neurotactin) |
| 823912 | 6.20E−03 | −0.96 | UBL3\|ubiquitin-like 3 |
| 854138 | 6.25E−03 | 1.01 | CSNK1E\|casein kinase 1, epsilon |
| 487697 | 6.26E−03 | −0.71 | CROT\|carnitine O-octanoyltransferase |
| 842765 | 6.27E−03 | −1.15 | PC326\|PC326 protein |
| 726597 | 6.35E−03 | −0.84 | Homo sapiens cDNA FLJ32642 fis, clone SYNOV2001144 |
| 172785 | 6.38E−03 | 0.60 | LOC51754\|NAG-5 protein |
| 898251 | 6.41E−03 | −1.55 | FLJ20727\|hypothetical protein FLJ20727 |
| 201976 | 6.44E−03 | −1.82 | ELF1\|E74-like factor 1 (ets domain transcription factor) |
| 42018 | 6.45E−03 | −1.09 | KIAA1468\|KIAA1468 protein |
| 78736 | 6.47E−03 | 0.94 | Homo sapiens clone 24877 mRNA sequence |
| 115292 | 6.48E−03 | −1.11 | DKFZp586C1924\|hypothetical protein DKFZp586C1924 |
| 22917 | 6.52E−03 | −0.66 | Homo sapiens mRNA; cDNA DKFZp761M0111 (from clone DKFZp761M0111) |
| 755228 | 6.60E−03 | 0.66 | DNM1\|dynamin 1 |
| 1075635 | 6.62E−03 | 0.85 | MTR1\|MLSN1- and TRP-related |
| 814826 | 6.66E−03 | −1.38 | ESTs |
| 322561 | 6.67E−03 | −0.95 | RPL31\|ribosomal protein L31 |
| 239862 | 6.68E−03 | −1.96 | KIAA0962\|KIAA0962 protein |
| 590544 | 6.69E−03 | −1.17 | MAPK9\|mitogen-activated protein kinase 9 |
| 897768 | 6.78E−03 | 0.70 | COL7A1\|collagen, type VII, alpha 1 (epidermolysis bullosa, dystrophic, dominant and recessive) |
| 376551 | 6.83E−03 | −1.67 | ETAA16\|ETAA16 protein |
| 2021956 | 6.84E−03 | 1.16 | LOC56930\|hypothetical protein from EUROIMAGE 1669387 |
| 877636 | 6.87E−03 | −1.18 | DCTN4\|dynactin 4 (p62) |
| 770579 | 6.87E−03 | 1.18 | CLDN3\|claudin 3 |
| 306318 | 6.91E−03 | 0.94 | ORC6L\|origin recognition complex, subunit 6 (yeast homolog)-like |
| 868308 | 7.01E−03 | −1.04 | ESTs, Highly similar to RS23_HUMAN 40S RIBOSOMAL PROTEIN S2 [H. sapiens] |
| 75415 | 7.02E−03 | −0.75 | HINT\|histidine triad nucleotide-binding protein |
| 823850 | 7.03E−03 | 0.71 | RAI14\|retinoic acid induced 14 |
| 1709786 | 7.05E−03 | −0.68 | TRPS1\|trichorhinophalangeal syndrome I |
| 2919651 | 7.12E−03 | 0.57 | PGLYRP\|peptidoglycan recognition protein |
| 965223 | 7.12E−03 | 1.59 | TK1\|thymidine kinase 1, soluble |
| 490251 | 7.13E−03 | −1.18 | PPP1R2\|protein phosphatase 1, regulatory (inhibitor) subunit 2 |
| 469172 | 7.14E−03 | −1.31 | SEC22C\|vesicle trafficking protein |
| 51981 | 7.15E−03 | −1.15 | GALNT2\|UDP-N-acetyl-alpha-D-galactosamine: polypeptide N-acetylgalactosaminyltransferase 2 (GalNAc- |
| | | | T2) |
| 1732922 | 7.19E−03 | 0.66 | Homo sapiens mRNA; cDNA DKFZp762H106 (from clone DKFZp762H106) |
| 288999 | 7.20E−03 | 0.90 | SPEC1\|small protein effector 1 of Cdc42 |
| 782339 | 7.23E−03 | 1.08 | PRKAB1\|protein kinase, AMP-activated, beta 1 non-catalytic subunit |
| 221632 | 7.34E−03 | 1.95 | EIF2B2\|eukaryotic translation initiation factor 2B, subunit 2 (beta, 39 kD) |
| 1605784 | 7.34E−03 | −1.26 | SYNE-2\|synaptic nuclei expressed gene 2 |
| 42070 | 7.40E−03 | 0.58 | NT5\|5′ nucleotidase (CD73) |
| 1637756 | 7.44E−03 | 1.07 | ENO1\|enolase 1, (alpha) |
| 37205 | 7.45E−03 | 0.72 | ESTs |
| 1625945 | 7.46E−03 | −0.98 | NDRG3\|N-myc downstream-regulated gene 3 |
| 32122 | 7.46E−03 | −1.04 | FLJ10210\|hypothetical protein FLJ10210 |
| 595297 | 7.48E−03 | −0.99 | SNAPAP\|SNARE associated protein snapin |
| 256680 | 7.50E−03 | −1.08 | BITE\|p10-binding protein |
| 1609372 | 7.50E−03 | −0.79 | RIPK3\|receptor-interacting serine-threonine kinase 3 |
| 1534719 | 7.50E−03 | 1.05 | MYO1D\|myosin ID |
| 2244561 | 7.52E−03 | 0.79 | CROC4\|transcriptional activator of the c-fos promoter |
| 70533 | 7.52E−03 | 1.21 | HPS\|Hermansky-Pudlak syndrome |
| 1562604 | 7.59E−03 | 1.25 | AP2A1\|adaptor-related protein complex 2, alpha 1 subunit |
| 470261-2 | 7.66E−03 | −0.61 | SMA5\|SMA5 |
| 781341 | 7.71E−03 | −1.02 | HSPE1\|heat shock 10 kD protein 1 (chaperonin 10) |
| 79565 | 7.72E−03 | −0.75 | FLJ22662\|hypothetical protein FLJ22662 |
| 52724 | 7.75E−03 | 0.98 | FLJ20241\|hypothetical protein FLJ20241 |
| 80727 | 7.75E−03 | 0.73 | ROR1\|receptor tyrosine kinase-like orphan receptor 1 |
| 377018 | 7.76E−03 | 1.00 | FLJ20850\|hypothetical protein FLJ20850 |
| 815507 | 7.77E−03 | −1.59 | |
| 841663 | 7.78E−03 | 0.95 | NARF\|nuclear prelamin A recognition factor |
| 147841 | 7.83E−03 | −0.82 | FLJ12287\|hypothetical protein FLJ12287 similar to semaphorins |
| 712559 | 7.91E−03 | −1.21 | SEC24A\|SEC24 (S. cerevisiae) related gene family, member A |
| 1031029 | 7.92E−03 | −2.65 | Homo sapiens cDNA FLJ32971 fis, clone TESTI2008847 |
| 66599 | 7.94E−03 | −0.38 | NAT1\|N-acetyltransferase 1 (arylamine N-acetyltransferase) |
| 789204 | 7.95E−03 | −1.20 | TLOC1\|translocation protein 1 |
| 71087 | 7.97E−03 | 1.11 | MAFF\|v-maf musculoaponeurotic fibrosarcoma (avian) oncogene family, protein F |
| 276816 | 7.98E−03 | 0.73 | KIAA1718\|KIAA1718 protein |
| 824915 | 8.00E−03 | 1.51 | CAPN10\|calpain 10 |
| 202901 | 8.07E−03 | 0.71 | VAV2\|vav 2 oncogene |
| 669375 | 8.10E−03 | 0.94 | DKK1\|dickkopf (Xenopus laevis) homolog 1 |
| 2116188 | 8.13E−03 | 0.83 | HDAC5\|histone deacetylase 5 |
| 814913 | 8.18E−03 | −0.83 | C11orf15\|chromosome 11 open reading frame 15 |
| 306013 | 8.19E−03 | −0.88 | MS4A1\|membrane-spanning 4-domains, subfamily A, member 1 |
| 950678 | 8.21E−03 | 1.05 | SREBF2\|sterol regulatory element binding transcription factor 2 |
| 2237279 | 8.25E−03 | −0.63 | LGI1\|leucine-rich, glioma inactivated 1 |
| 33076 | 8.33E−03 | −0.54 | LOC56994\|cholinephosphotransferase 1 |
| 469924 | 8.35E−03 | 1.07 | PCTP\|phosphatidylcholine transfer protein |
| 190021 | 8.40E−03 | 1.25 | PIASY\|protein inhibitor of activated STAT protein PIASy |
| 769579 | 8.42E−03 | 0.81 | MAP2K2\|mitogen-activated protein kinase kinase 2 |
| 1558832 | 8.44E−03 | −1.08 | MAT2B\|methionine adenosyltransferase II, beta |
| 772455 | 8.45E−03 | −1.02 | PPP4C\|protein phosphatase 4 (formerly X), catalytic subunit |
| 30673 | 8.49E−03 | −0.51 | KIAA1022\|cortactin SH3 domain-binding protein |
| 417884 | 8.49E−03 | −0.60 | Homo sapiens cDNA FLJ12052 fis, clone HEMBB1002042, moderately similar to |
| | | | CYTOCHROME P450 4C1 (EC 1.14.14.1) |
| 757435 | 8.49E−03 | −0.49 | NKX3A\|NK homeobox (Drosophila), family 3, A |
| 230910 | 8.50E−03 | 1.13 | |
| 1559198 | 8.52E−03 | −0.95 | Homo sapiens cDNA FLJ14923 fis, clone PLACE1008244, weakly similar to VEGETATIBLE |
| | | | INCOMPATIBILITY PROTEIN HET-E-1 |
| 809353 | 8.58E−03 | 0.99 | IRF3\|interferon regulatory factor 3 |
| 564981 | 8.66E−03 | 0.78 | Homo sapiens, Similar to RIKEN cDNA 2810433K01 gene, clone MGC: 10200 IMAGE: 3909951, mRNA, |
| | | | complete cds |
| 786048 | 8.66E−03 | 0.90 | E2F4\|E2F transcription factor 4, p107/p130-binding |
| 209066 | 8.67E−03 | 0.62 | STK15\|serine/threonine kinase 15 |
| 2214020 | 8.68E−03 | −1.36 | GRIN2D\|glutamate receptor, ionotropic, N-methyl D-aspartate 2D |
| 815276 | 8.68E−03 | 1.23 | NUP62\|nucleoporin 62 kD |
| 813845 | 8.75E−03 | −0.94 | RNUT1\|RNA, U transporter 1 |
| 471568 | 8.76E−03 | 0.89 | HN1\|hematological and neurological expressed 1 |
| 845419 | 8.77E−03 | 1.04 | FANCA\|Fanconi anemia, complementation group A |
| 1631713 | 8.78E−03 | −1.02 | NEDD5\|neural precursor cell expressed, developmentally down-regulated 5 |
| 2504698 | 8.83E−03 | 1.10 | ARRB2\|arrestin, beta 2 |
| 1911463 | 8.90E−03 | −1.36 | ESTs |
| 1475028 | 8.94E−03 | −0.77 | RPS27\|ribosomal protein S27 (metallopanstimulin 1) |
| 502161 | 8.99E−03 | 0.75 | APPBP1\|amyloid beta precursor protein-binding protein 1, 59 kD |
| 509459 | 9.13E−03 | 0.99 | Homo sapiens cDNA FLJ14241 fis, clone OVARC1000533 |
| 712049 | 9.14E−03 | −1.16 | IL24\|interleukin 24 |
| 785549 | 9.16E−03 | −1.28 | KIAA1902\|KIAA1902 protein |
| 809421 | 9.17E−03 | −0.85 | PCBD\|6-pyruvoyl-tetrahydropterin synthase/dimerization cofactor of hepatocyte nuclear factor 1 alpha |
| | | | (TCF1) |
| 154493 | 9.20E−03 | −0.89 | IFI41\|interferon-induced protein 41, 30 kD |
| 130845 | 9.25E−03 | −1.15 | PWP1\|nuclear phosphoprotein similar to S. cerevisiae PWP1 |
| 2508044 | 9.30E−03 | 0.80 | HP\|haptoglobin |
| 2013908 | 9.32E−03 | −1.07 | |
| 2054122 | 9.43E−03 | −0.39 | SLC11A3\|solute carrier family 11 (proton-coupled divalent metal ion transporters), member 3 |
| 812159 | 9.46E−03 | 1.15 | FLJ20337\|hypothetical protein FLJ20337 |
| 742695 | 9.49E−03 | −0.90 | Homo sapiens cDNA FLJ31534 fis, clone NT2RI2000671 |
| 69002 | 9.50E−03 | 0.41 | ANGPTL4\|angiopoietin-like 4 |
| 32812 | 9.56E−03 | −0.98 | BCAS2\|breast carcinoma amplified sequence 2 |
| 753038 | 9.62E−03 | 0.76 | KIFC3\|kinesin family member C3 |
| 704299 | 9.74E−03 | 1.10 | TAZ\|tafazzin (cardiomyopathy, dilated 3A (X-linked); endocardial fibroelastosis 2; Barth syndrome) |
| 815501 | 9.74E−03 | 0.79 | MGC2721\|hypothetical protein MGC2721 |
| 3208314 | 9.75E−03 | −0.58 | GPR27\|G protein-coupled receptor 27 |
| 758343 | 9.78E−03 | 1.01 | PPIF\|peptidylprolyl isomerase F (cyclophilin F) |
| 361587 | 9.80E−03 | −0.48 | KIAA1789\|KIAA1789 protein |
| 814951 | 9.81E−03 | −1.26 | Homo sapiens, RIKEN cDNA 2310005G07 gene, clone MGC: 10049 IMAGE: 3890955, mRNA, |
| | | | complete cds |
| 323780 | 9.82E−03 | 1.34 | Homo sapiens cDNA FLJ11177 fis, clone PLACE1007402 |
| 1603404 | 9.82E−03 | −0.76 | LR8\|LR8 protein |
| 132637 | 9.86E−03 | −0.97 | GCA\|grancalcin, EF-hand calcium-binding protein |
| 131653 | 9.87E−03 | −1.63 | MRPS12\|mitochondrial ribosomal protein S12 |
| 897669 | 9.87E−03 | 1.08 | PRKCSH\|protein kinase C substrate 80K-H |
| 49273 | 9.89E−03 | 0.78 | SLC27A4\|solute carrier family 27 (fatty acid transporter), member 4 |
| 530875 | 9.97E−03 | −0.37 | TKT\|transketolase (Wernicke-Korsakoff syndrome) |

TABLE 5
297 gene subset of genes in Table 4

| Clone_ID | GB_ID | Unigene_ID |
|---|---|---|
| 22917 | AL137346 | Hs.13299 |
| 23772 | NM_006767 | Hs.78788 |
| 23831 | NM_005165 | Hs.155247 |
| 26314 | NM_007269 | Hs.8813 |
| 26507 | AB002304 | Hs.356290 |
| 26811 | NM_003401 | Hs.150930 |
| 26856 | NM_004475 | Hs.184488 |
| 30673 | AB028945 | Hs.12696 |
| 30673 | AF141901 | Hs.12696 |
| 32122 | NM_018027 | Hs.183639 |
| 32684 | NM_000994 | Hs.169793 |
| 32812 | NM_005872 | Hs.22960 |
| 33076 | NM_020244 | Hs.171889 |
| 34852 | NM_001166 | Hs.289107 |
| 38244 | AL109693 | Hs.301338 |
| 39677 | NM_018184 | Hs.104222 |
| 40173 | AB018350 | Hs.101474 |
| 41647 | NM_007050 | Hs.225952 |
| 42018 | AB040901 | Hs.23542 |
| 42070 | NM_002526 | Hs.153952 |
| 44443 | NM_004757 | Hs.333513 |
| 47795 | NM_007146 | Hs.6557 |
| 49273 | NM_005094 | Hs.248953 |
| 50794 | NM_003434 | Hs.78434 |
| 51328 | L22005 | Hs.76932 |
| 51469 | AK001980 | Hs.24284 |
| 51981 | NM_000972 | Hs.99858 |
| 52724 | AK000482 | Hs.181780 |
| 52724 | NM_017721 | Hs.181780 |
| 66599 | NM_000662 | Hs.155956 |
| 68345 | NM_002224 | Hs.77515 |
| 68557 | NM_001443 | Hs.351719 |
| 69002 | NM_016109 | Hs.9613 |
| 70533 | NM_000195 | Hs.83951 |
| 71087 | NM_012323 | Hs.51305 |
| 75415 | NM_005340 | Hs.256697 |
| 78736 | AF131821 | Hs.3964 |
| 80727 | NM_005012 | Hs.274243 |
| 83653 | NM_014167 | Hs.90527 |
| 84068 | AK001913 | Hs.7100 |
| 85409 | NM_003851 | Hs.5710 |
| 85614 | NM_015344 | Hs.11000 |
| 109316 | NM_001085 | Hs.234726 |
| 124046 | NM_012279 | Hs.181012 |
| 126221 | NM_003288 | Hs.154718 |
| 128426 | AF156603 | Hs.285681 |
| 130153 | NM_003169 | Hs.70186 |
| 130845 | NM_007062 | Hs.172589 |
| 132637 | NM_012198 | Hs.79381 |
| 134270 | U68494 | Hs.24385 |
| 135303 | NM_018480 | Hs.24371 |
| 135503 | NM_014299 | Hs.278675 |
| 137836 | NM_007217 | Hs.28866 |
| 138788 | NM_000949 | Hs.1906 |
| 140574 | NM_002996 | Hs.80420 |
| 140951 | NM_004924 | Hs.182485 |
| 150137 | NM_014043 | Hs.11449 |
| 150897 | NM_014256 | Hs.69009 |
| 154493 | NM_004509 | Hs.38125 |
| 154493 | NM_004510 | Hs.38125 |
| 155920 | NM_018028 | Hs.127240 |
| 165828 | NM_013241 | Hs.95231 |
| 166199 | NM_001619 | Hs.83636 |
| 172785 | NM_016446 | Hs.8087 |
| 190021 | NM_015897 | Hs.105779 |
| 198874 | NM_018273 | Hs.19039 |
| 201976 | M82882 | Hs.154365 |
| 202577 | NM_006895 | Hs.81182 |
| 221632 | NM_014239 | Hs.170001 |
| 229901 | NM_001334 | Hs.75262 |
| 235056 | AF070535 | Hs.78019 |
| 239862 | AB023179 | Hs.9059 |
| 242706 | NM_014145 | Hs.3576 |
| 244307 | M16006 | Hs.82085 |
| 244307 | NM_000602 | Hs.82085 |
| 262739 | NM_007190 | Hs.300208 |
| 262916 | NM_002706 | Hs.5687 |
| 267590 | NM_012295 | Hs.7840 |
| 277999 | AL080129 | Hs.225841 |
| 279085 | NM_004145 | Hs.159629 |
| 279800 | NM_007159 | Hs.4007 |
| 280249 | NM_003709 | Hs.21599 |
| 288999 | NM_020239 | Hs.22065 |
| 295781 | AL035369 | Hs.33922 |
| 296190 | AB002319 | Hs.8663 |
| 299388 | NM_005796 | Hs.151734 |
| 306013 | X07203 | Hs.89751 |
| 306318 | NM_014321 | Hs.49760 |
| 306933 | AF131828 | Hs.7961 |
| 307933 | NM_002492 | Hs.19236 |
| 322511 | AL080078 | Hs.85335 |
| 322561 | NM_000993 | Hs.184014 |
| 323693 | NM_001283 | Hs.57600 |
| 325515 | AB037791 | Hs.29716 |
| 345423 | NM_015387 | Hs.107942 |
| 365919 | NM_004602 | Hs.6113 |
| 365919 | NM_017453 | Hs.6113 |
| 365919 | NM_017454 | Hs.6113 |
| 376551 | NM_019002 | Hs.82664 |
| 377018 | NM_017967 | Hs.30783 |
| 469172 | NM_004206 | Hs.12942 |
| 469924 | AF151638 | Hs.285218 |
| 469924 | NM_021213 | Hs.285218 |
| 471568 | NM_016185 | Hs.109706 |
| 487297 | NM_006366 | Hs.296341 |
| 487697 | AF073770 | Hs.12743 |
| 487697 | NM_021151 | Hs.12743 |
| 488505 | NM_005629 | Hs.187958 |
| 490251 | NM_006241 | Hs.267819 |
| 490753 | NM_017812 | Hs.6693 |
| 491053 | NM_006321 | Hs.241558 |
| 502151 | NM_004207 | Hs.85838 |
| 502161 | NM_003905 | Hs.61828 |
| 502891 | NM_018352 | Hs.267446 |
| 504826 | NM_003201 | Hs.75133 |
| 529147 | NM_004896 | Hs.67052 |
| 530875 | NM_001064 | Hs.89643 |
| 530875 | NM_005516 | Hs.89643 |
| 530954 | AL117457 | Hs.180141 |
| 586650 | NM_004955 | Hs.25450 |
| 587847 | NM_002083 | Hs.2704 |
| 590338 | NM_015920 | Hs.108957 |
| 595297 | NM_012437 | Hs.32018 |
| 628357 | NM_001104 | Hs.1216 |
| 666169 | NM_000254 | Hs.82283 |
| 669375 | NM_012242 | Hs.40499 |
| 685516 | NM_014373 | Hs.97101 |
| 686552 | AF020762 | Hs.6831 |
| 704299 | NM_000116 | Hs.79021 |
| 712049 | NM_006850 | Hs.315463 |
| 712559 | AJ131244 | Hs.211612 |
| 724615 | NM_001269 | Hs.84746 |
| 725395 | NM_004223 | Hs.169895 |
| 739191 | NM_005096 | Hs.9568 |
| 741474 | NM_000175 | Hs.279789 |
| 742007 | D63480 | Hs.278634 |
| 744047 | NM_005030 | Hs.77597 |
| 745360 | NM_003642 | Hs.13340 |
| 753038 | NM_005550 | Hs.23131 |
| 754537 | AK001091 | Hs.274415 |
| 754582 | NM_014210 | Hs.70499 |
| 755228 | NM_004408 | Hs.166161 |
| 755578 | NM_003486 | Hs.184601 |
| 756662 | AB023160 | Hs.352535 |
| 756662 | NM_013325 | Hs.352535 |
| 757435 | NM_006167 | Hs.55999 |
| 758318 | NM_012175 | Hs.16577 |
| 758343 | NM_005729 | Hs.173125 |
| 767068 | AL117452 | Hs.44155 |
| 767495 | NM_000168 | Hs.72916 |
| 767753 | NM_000449 | Hs.166891 |
| 769004 | NM_016195 | Hs.240 |
| 769537 | NM_001398 | Hs.196176 |
| 769579 | L11285 | Hs.72241 |
| 769712 | NM_005255 | Hs.153227 |
| 770518 | AL080109 | Hs.295112 |
| 770518 | NM_014833 | Hs.295112 |
| 770579 | NM_001306 | Hs.25640 |
| 770588 | AF000560 | Hs.79531 |
| 770835 | NM_000056 | Hs.1265 |
| 772455 | NM_002720 | Hs.2903 |
| 774446 | NM_001124 | Hs.394 |
| 780947 | NM_002691 | Hs.99890 |
| 781222 | NM_004740 | Hs.75822 |
| 784150 | NM_006868 | Hs.223025 |
| 785459 | AJ010306 | Hs.149098 |
| 785459 | NM_006932 | Hs.149098 |
| 786048 | NM_001950 | Hs.108371 |
| 786674 | Z31560 | Hs.816 |
| 788511 | NM_002953 | Hs.149957 |
| 788745 | NM_006571 | Hs.39913 |
| 789204 | NM_003262 | Hs.8146 |
| 795893 | NM_014330 | Hs.76556 |
| 795936 | NM_004622 | Hs.75066 |
| 796114 | NM_012238 | Hs.31176 |
| 796255 | AL049705 | Hs.247324 |
| 796694 | NM_001168 | Hs.1578 |
| 809353 | NM_001571 | Hs.75254 |
| 809421 | NM_000281 | Hs.3192 |
| 810063 | NM_005262 | Hs.27184 |
| 810391 | NM_007312 | Hs.75619 |
| 810983 | NM_015492 | Hs.17936 |
| 811029 | AB002363 | Hs.190452 |
| 811790 | NM_014044 | Hs.13370 |
| 811888 | AL050171 | Hs.5306 |
| 812159 | NM_017772 | Hs.26898 |
| 813490 | NM_014325 | Hs.17377 |
| 813845 | NM_005701 | Hs.21577 |
| 814285 | NM_018368 | Hs.339833 |
| 815057 | NM_018169 | Hs.236844 |
| 815235 | NM_014329 | Hs.75682 |
| 815276 | NM_012346 | Hs.9877 |
| 815276 | NM_016553 | Hs.9877 |
| 815535 | NM_000356 | Hs.301266 |
| 823850 | AB037755 | Hs.15165 |
| 823912 | NM_007106 | Hs.173091 |
| 824352 | NM_002874 | Hs.178658 |
| 824510 | NM_016062 | Hs.9825 |
| 824915 | NM_021251 | Hs.112218 |
| 825176 | NM_018374 | Hs.3542 |
| 826256 | NM_003272 | Hs.15791 |
| 826286 | NM_014652 | Hs.158497 |
| 840506 | NM_016085 | Hs.9527 |
| 840865 | NM_002356 | Hs.75607 |
| 841663 | AL137729 | Hs.256526 |
| 841663 | NM_012336 | Hs.256526 |
| 842765 | NM_018442 | Hs.279882 |
| 842968 | NM_001211 | Hs.36708 |
| 845419 | NM_000135 | Hs.284153 |
| 854138 | NM_001894 | Hs.79658 |
| 855800 | NM_002726 | Hs.86978 |
| 855872 | NM_002525 | Hs.4099 |
| 856164 | NM_015032 | Hs.168625 |
| 856164 | NM_015928 | Hs.168625 |
| 860000 | NM_002914 | Hs.139226 |
| 877636 | NM_016221 | Hs.180952 |
| 882488 | NM_005652 | Hs.100030 |
| 884438 | NM_006164 | Hs.155396 |
| 884455 | NM_012094 | Hs.31731 |
| 897164 | NM_001903 | Hs.178452 |
| 897767 | NM_004818 | Hs.168103 |
| 897768 | NM_000094 | Hs.1640 |
| 897971 | NM_016451 | Hs.3059 |
| 898251 | NM_017944 | Hs.300700 |
| 898312 | NM_004295 | Hs.8375 |
| 950667 | NM_020386 | Hs.36761 |
| 950678 | NM_004599 | Hs.108689 |
| 965223 | NM_003258 | Hs.105097 |
| 1030351 | NM_005409 | Hs.103982 |
| 1075635 | AJ270996 | Hs.272287 |
| 1404396 | Z26649 | Hs.37121 |
| 1416782 | NM_001823 | Hs.173724 |
| 1466237 | NM_015641 | Hs.165986 |
| 1474164 | NM_019108 | Hs.10116 |
| 1475028 | NM_001030 | Hs.195453 |
| 1475738 | NM_001028 | Hs.113029 |
| 1500241 | AL137572 | Hs.48778 |
| 1506046 | NM_018231 | Hs.10499 |
| 1534719 | AB018270 | Hs.39871 |
| 1558832 | AF182814 | Hs.54642 |
| 1592530 | AL117458 | Hs.323432 |
| 1592530 | AL137514 | Hs.323432 |
| 1592530 | NM_016291 | Hs.323432 |
| 1601601 | NM_000758 | Hs.1349 |
| 1603404 | NM_014020 | Hs.190161 |
| 1603583 | NM_003022 | Hs.14368 |
| 1605784 | AL080133 | Hs.57749 |
| 1605784 | AL117404 | Hs.57749 |
| 1609372 | NM_006871 | Hs.268551 |
| 1631713 | NM_004404 | Hs.155595 |
| 1635581 | NM_016539 | Hs.105463 |
| 1635618 | NM_014931 | Hs.72172 |
| 1635695 | NM_015044 | Hs.155546 |
| 1636166 | AB014568 | Hs.5898 |
| 1637282 | NM_000189 | Hs.198427 |
| 1637756 | M55914 | Hs.254105 |
| 1637756 | NM_001428 | Hs.254105 |
| 1693357 | NM_001956 | Hs.1407 |
| 1702742 | NM_003486 | Hs.184601 |
| 1709786 | NM_014112 | Hs.26102 |
| 1732922 | AL162069 | Hs.140978 |
| 1733935 | NM_004941 | Hs.171872 |
| 1734309 | AF262992 | Hs.123159 |
| 1737724 | NM_002319 | Hs.125742 |
| 1752548 | NM_019098 | Hs.154433 |
| 1871423 | NM_004661 | Hs.153546 |
| 1882051 | NM_017657 | Hs.7942 |
| 1894519 | AL157464 | Hs.48827 |
| 1903067 | NM_017438 | Hs.50748 |
| 1908840 | NM_003450 | Hs.155204 |
| 1913943 | NM_002032 | Hs.62954 |
| 1926249 | AF052087 | Hs.128425 |
| 1926575 | NM_001265 | Hs.77399 |
| 1947804 | NM_016381 | Hs.278408 |
| 2009779 | NM_004703 | Hs.326056 |
| 2015148 | NM_014030 | Hs.318339 |
| 2016426 | AB014564 | Hs.22616 |
| 2018808 | NM_005040 | Hs.75693 |
| 2054122 | NM_014585 | Hs.5944 |
| 2062825 | NM_014902 | Hs.177425 |
| 2116188 | NM_005474 | Hs.9028 |
| 2125819 | NM_004324 | Hs.159428 |
| 2237279 | NM_005097 | Hs.194704 |
| 2239290 | NM_000609 | Hs.237356 |
| 2239290 | U16752 | Hs.237356 |
| 2244561 | NM_006365 | Hs.322469 |
| 2306919 | NM_012243 | Hs.159322 |
| 2307119 | NM_001566 | Hs.32944 |
| 2307119 | NM_004027 | Hs.32944 |
| 2313673 | AL080084 | Hs.348996 |
| 2313673 | NM_016040 | Hs.348996 |
| 2313921 | NM_002491 | Hs.109760 |
| 2502722 | NM_014622 | Hs.152944 |
| 2504698 | NM_004313 | Hs.18142 |
| 2508044 | NM_005143 | Hs.75990 |
| 2919651 | NM_005091 | Hs.137583 |
| 3208314 | NM_018971 | Hs.278283 |

### Example IV

### Molecular Signatures of Four Additional Breast Cancer Subtypes

Frozen breast cancer samples from 247 patients were expression profiled and classified into four subtypes (A, B, C, and D) based on the expression of gene sequences in correlation with survival outcomes of the patients from whom the samples were obtained.

Within the set of 247 samples, 143 were ER+ via a biomarker test. Within this set of 41, microdissection was used to obtain breast cancer cells for identification of a molecular signature (i.e., expression of genes) that differentially categorized the ER+ group into subtypes A and B. The remaining samples were microdissected to obtain cells for identification of subtypes C and D.

The 50 genes which are overexpressed in relation to each of subtypes A, B, C, and D are shown in Tables 6, 7, 8, and 9, respectively. The number of samples classified into subtypes A, B, C, and D are 86, 57, 70, and 34, respectively.

Subtypes A and B are both subtypes of ER+ samples with significantly different survival outcomes as shown in FIG. 3. Subtype C samples are ER− and so may be viewed as, as well as used as, gene sequences the overexpression of which are indicative of ER−status. The survival outcomes of patients with subtype C samples are shown in FIG. 3. It is interesting to note that subtype B samples are from patients with survival similar to that of subtype C (patients whose tumors were ER negative). As such, an additional aspect of the invention is the treatment of patients with subtype B breast cancer cells in the manner of treating patients with cells having an ER negative phenotype.

Subtype D samples are independent of ER status and thus contain samples that may be ER+ or ER−. The survival outcomes of patients with subtype C samples are also shown in FIG. 3. Similar to subtype B as discussed above, the invention provides for the treatment of patients with subtype D breast cancer cells in the manner of treating patients with cells having an ER negative phenotype.

TABLE 6
50 gene sequences which define Subtype A

| P values | | |
|---|---|---|
| (Wilcoxon Test) | GeneID | Description |
| 6.40592E−18 | AW473119 | ESR1\|estrogen receptor 1 |
| 4.98711E−17 | AA130089 | ESTs |
| 5.56867E−17 | AL049265 | Homo sapiens mRNA; cDNA DKFZp564F053 (from clone DKFZp564F053) |
| 2.14044E−16 | AL360204 | Homo sapiens mRNA full length insert cDNA clone EUROIMAGE 980547 |
| 3.93903E−16 | AK000158 | FLJ20151\|hypothetical protein FLJ20151 |
| 8.60498E−16 | AI457338 | Homo sapiens cDNA FLJ33115 fis, clone TRACH2001314 |
| 1.02633E−15 | AL157499 | Homo sapiens mRNA; cDNA DKFZp434N2412 (from clone |
| | | DKFZp434N2412) |
| 1.0264E−15 | AK024999 | Homo sapiens cDNA: FLJ21346 fis, clone COL02705 |
| 1.14067E−15 | AF131785 | KIAA0882\|KIAA0882 protein |
| 1.51026E−15 | AW265341 | ESTs |
| 1.56394E−15 | AI439798 | FGD3\|FGD1 family, member 3 |
| 1.61961E−15 | AK022441 | Homo sapiens cDNA FLJ12379 fis, clone MAMMA1002554 |
| 1.86262E−15 | BC008317 | LIV-1\|LIV-1 protein, estrogen regulated |
| 1.92875E−15 | BC014948 | MLPH\|melanophilin |
| 3.99501E−15 | AF176012 | JDP1\|J domain containing protein 1 |
| 4.58544E−15 | AI200852 | ESTs |
| 5.2605E−15 | AW015443 | ESTs, Weakly similar to JE0350 Anterior gradient-2 [H. sapiens] |
| 6.24497E−15 | R49089 | ESTs, Moderately similar to T12539 hypothetical protein DKFZp434J154.1 |
| | | [H. sapiens] |
| 6.68731E−15 | AW300348 | Homo sapiens ovarian cancer-related protein 2 (OCR2) mRNA, complete cds |
| 8.4916E−15 | AF070632 | Homo sapiens clone 24405 mRNA sequence |
| 1.27628E−14 | AI277016 | ESTs |
| 1.27636E−14 | BF433570 | ESTs |
| 1.3202E−14 | AL133622 | KIAA0876\|KIAA0876 protein |
| 1.34262E−14 | BE967259 | BCL2\|B-cell CLL/lymphoma 2 |
| 1.78871E−14 | AI364725 | KIAA0239\|KIAA0239 protein |
| 1.91317E−14 | BC007997 | RERG\|RAS-like, estrogen-regulated, growth-inhibitor |
| 2.50201E−14 | AY009106 | DKFZP434I092\|DKFZP434I092 protein |
| 3.61137E−14 | AK000269 | FLJ20262\|hypothetical protein FLJ20262 |
| 4.05649E−14 | AI263695 | NME5\|non-metastatic cells 5, protein expressed in (nucleoside-diphosphate |
| | | kinase) |
| 4.55599E−14 | AL050116 | Homo sapiens mRNA; cDNA DKFZp586A131 (from clone DKFZp586A131) |
| 4.8679E−14 | BF110928 | ESTs, Weakly similar to I38022 hypothetical protein [H. sapiens] |
| 7.97977E−14 | AF035282 | C1orf21\|chromosome 1 open reading frame 21 |
| 8.52063E−14 | AA775255 | ANKHZN\|ANKHZN protein |
| 9.09746E−14 | AF052504 | RNB6\|RNB6 |
| 1.00347E−13 | AI912086 | Homo sapiens cDNA FLJ30744 fis, clone FEBRA2000378 |
| 1.07127E−13 | BC013732 | NAT1\|N-acetyltransferase 1 (arylamine N-acetyltransferase) |
| 1.1068E−13 | AF007153 | Homo sapiens clone 23736 mRNA sequence |
| 1.14343E−13 | AK058158 | Homo sapiens cDNA FLJ25429 fis, clone TST05630 |
| 1.34564E−13 | BC017701 | AD036\|AD036 protein |
| 1.39009E−13 | BF129497 | EST |
| 1.6349E−13 | NM_020974 | CEGP1\|CEGP1 protein |
| 1.80162E−13 | AL136926 | DKFZP586M1120\|hypothetical protein DKFZp586M1120 |
| 1.98501E−13 | NM_016613 | LOC51313\|AD021 protein |
| 2.05012E−13 | AI128582 | ESTs |
| 2.11732E−13 | AA826324 | Homo sapiens cDNA FLJ32320 fis, clone PROST2003537 |
| 2.25829E−13 | BC010607 | Homo sapiens, clone MGC: 18216 IMAGE: 4156235, mRNA, complete cds |
| 3.01538E−13 | AK027148 | FLJ23495\|hypothetical protein FLJ23495 |
| 4.2846E−13 | AI382972 | TPBG\|trophoblast glycoprotein |
| 4.71356E−13 | BC017338 | FUCA1\|fucosidase, alpha-L-1, tissue |
| 5.02267E−13 | BC000809 | TCEAL1\|transcription elongation factor A (SII)-like 1 |

TABLE 7
50 gene sequences which define Subtype B

| P values | | |
|---|---|---|
| (Wilcoxon Test) | GeneID | Description |
| 1.38458E−08 | BC007659 | NQO1\|NAD(P)H dehydrogenase, quinone 1 |
| 1.14979E−07 | NM_012134 | LMOD1\|leiomodin 1 (smooth muscle) |
| 1.664E−07 | BF436656 | MFAP4\|microfibrillar-associated protein 4 |
| 2.33563E−07 | BC010690 | FLJ14529\|hypothetical protein FLJ14529 |
| 5.84863E−07 | AF035408 | CILP\|cartilage intermediate layer protein, nucleotide pyrophosphohydrolase |
| 5.99703E−07 | NM_014890 | DOC1\|downregulated in ovarian cancer 1 |
| 8.49583E−07 | AF068651 | LDB2\|LIM domain binding 2 |
| 1.32045E−06 | BE671609 | ESTs, Weakly similar to T28770 hypothetical protein W03D2.1 - |
| | | Caenorhabditis elegans [C. elegans] |
| 1.3529E−06 | BC005939 | PTGDS\|prostaglandin D2 synthase (21 kD, brain) |
| 1.4201E−06 | BC011535 | DKFZP566K1924\|DKFZP566K1924 protein |
| 1.45481E−06 | BC008750 | NDN\|necdin homolog (mouse) |
| 1.52693E−06 | AI378647 | ESTs |
| 1.94159E−06 | AI499501 | ESTs, Weakly similar to FMOD_HUMAN FIBROMODULIN PRECURSOR |
| | | [H. sapiens] |
| 2.24009E−06 | AL079279 | Homo sapiens mRNA full length insert cDNA clone EUROIMAGE 248114 |
| 2.83756E−06 | AJ295149 | LOC64174\|putative dipeptidase |
| 3.42268E−06 | AK024551 | FLJ20898\|hypothetical protein FLJ20898 |
| 3.75687E−06 | AI095484 | Homo sapiens cDNA FLJ32163 fis, clone PLACE6000371 |
| 3.80068E−06 | U67784 | RDC1\|G protein-coupled receptor |
| 4.2186E−06 | AF035269 | PS-PLA1\|phosphatidylserine-specific phospholipase A1alpha |
| 4.31724E−06 | AF137027 | TCL1B\|T-cell leukemia/lymphoma 1B |
| 4.52117E−06 | BC012160 | TNFRSF7\|tumor necrosis factor receptor superfamily, member 7 |
| 4.52117E−06 | BC001232 | C6orf32\|chromosome 6 open reading frame 32 |
| 5.55831E−06 | NM_003734 | AOC3\|amine oxidase, copper containing 3 (vascular adhesion protein 1) |
| 5.55831E−06 | AI952055 | ESTs |
| 6.15839E−06 | BC018650 | EDG1\|endothelial differentiation, sphingolipid G-protein-coupled receptor, 1 |
| 7.3812E−06 | BC016964 | Homo sapiens, clone MGC: 21621 IMAGE: 4181577, mRNA, complete cds |
| 7.63505E−06 | AL136805 | KIAA1474\|KIAA1474 protein |
| 7.80877E−06 | NM_001773 | CD34\|CD34 antigen |
| 7.80877E−06 | BC009698 | APOC1\|apolipoprotein C-I |
| 8.35283E−06 | BC015694 | KIAA1607\|KIAA1607 protein |
| 8.54208E−06 | R42463 | ENTPD1\|ectonucleoside triphosphate diphosphohydrolase 1 |
| 9.34072E−06 | AI470943 | ESTs |
| 1.06731E−05 | AJ238044 | BDKRB1\|bradykinin receptor B1 |
| 1.09121E−05 | X86163 | BDKRB2\|bradykinin receptor B2 |
| 1.14056E−05 | AI754777 | ESTs |
| 1.16602E−05 | AW024539 | ESTs |
| 1.1789E−05 | AW295374 | Homo sapiens cDNA FLJ11422 fis, clone HEMBA1001008 |
| 1.27335E−05 | AA749213 | GMFG\|glia maturation factor, gamma |
| 1.33048E−05 | BC016755 | HFL1\|H factor (complement)-like 1 |
| 1.35995E−05 | AI671590 | C11orf21\|chromosome 11 open reading frame 21 |
| 1.48413E−05 | NM_001504 | GPR9\|G protein-coupled receptor 9 |
| 1.51683E−05 | AW874252 | ESTs, Moderately similar to PBK1 protein [H. sapiens] |
| 1.51686E−05 | AF052094 | EPAS1\|endothelial PAS domain protein 1 |
| 1.72788E−05 | NM_002405 | MFNG\|manic fringe homolog (Drosophila) |
| 1.76565E−05 | AK025307 | CPT1A\|carnitine palmitoyltransferase I, liver |
| 1.80417E−05 | NM_000609 | SDF1\|stromal cell-derived factor 1 |
| 1.80421E−05 | NM_004419 | DUSP5\|dual specificity phosphatase 5 |
| 1.96658E−05 | BI492073 | ITM2A\|integral membrane protein 2A |
| 2.00929E−05 | X56210 | HFL2\|H factor (complement)-like 2 |
| 2.05284E−05 | AF131817 | Homo sapiens clone 25023 mRNA sequence |

TABLE 8
50 gene sequences which define Subtype C

| P values | | |
|---|---|---|
| (Wilcoxon Test) | GeneID | Description |
| 1.12657E−20 | AW450675 | ESTs |
| 1.96271E−20 | AW139831 | Homo sapiens cDNA FLJ11796 fis, clone HEMBA1006158, highly similar to |
| | | Homo sapiens transcription factor forkhead-like 7 (FKHL7) gene |
| 1.96289E−20 | NM_014211 | GABRP\|gamma-aminobutyric acid (GABA) A receptor, pi |
| 6.14853E−20 | AW004032 | LOC56963\|hypothetical protein from EUROIMAGE 363668 |
| 6.41109E−20 | NM_001453 | FOXC1\|forkhead box C1 |
| 7.58367E−20 | N31940 | ESTs, Weakly similar to 2004399A chromosomal protein [H. sapiens] |
| 2.06095E−19 | NM_005044 | PRKX\|protein kinase, X-linked |
| 3.82617E−19 | AF257472 | C21orf68\|chromosome 21 open reading frame 68 |
| 3.98699E−19 | AI567843 | ESTs, Weakly similar to JC5314 CDC28/cdc2-like kinase associating arginine- |
| | | serine cyclophilin [H. sapiens] |
| 4.15413E−19 | AI160174 | ESTs |
| 5.09939E−19 | AW140023 | FLJ13204\|hypothetical protein FLJ13204 |
| 5.5344E−19 | AI800206 | STAC\|src homology three (SH3) and cysteine rich domain |
| 7.0715E−19 | AA767129 | PRKY\|protein kinase, Y-linked |
| 2.02758E−18 | AJ404611 | BCL11A\|B-cell CLL/lymphoma 11A (zinc finger protein) |
| 2.28777E−18 | AI804716 | ESTs |
| 2.28777E−18 | AJ010277 | TBX19\|T-box 19 |
| 2.91023E−18 | BC017913 | ART3\|ADP-ribosyltransferase 3 |
| 3.15313E−18 | AAI56097 | ESTs, Weakly similar to LKHU proteoglycan link protein precursor [H. sapiens] |
| 3.69992E−18 | NM_032047 | B3GNT5\|UDP-GlcNAc: betaGal beta-1,3-N-acetylglucosaminyltransferase 5 |
| 4.0074E−18 | AF118070 | DKFZp762A227\|hypothetical protein DKFZp762A227 |
| 4.0074E−18 | AK026733 | Homo sapiens cDNA: FLJ23080 fis, clone LNG06052 |
| 4.5165E−18 | AW071804 | ESTs |
| 4.5165E−18 | AB037813 | DKFZp762K222\|hypothetical protein DKFZp762K222 |
| 5.51045E−18 | BC017352 | TRIM29\|tripartite motif-containing 29 |
| 5.73373E−18 | AW204371 | DSC2\|desmocollin 2 |
| 6.2074E−18 | BC000045 | TONDU\|TONDU |
| 9.59111E−18 | S72493 | KRT16\|keratin 16 (focal non-epidermolytic palmoplantar keratoderma) |
| 1.79795E−17 | AW206460 | KIAA0481\|KIAA0481 gene product |
| 1.79795E−17 | NM_002852 | PTX3\|pentaxin-related gene, rapidly induced by IL-1 beta |
| 2.65568E−17 | AK025251 | CHST3\|carbohydrate (chondroitin 6) sulfotransferase 3 |
| 2.761E−17 | AK026946 | FLJ23293\|likely ortholog of mouse ADP-ribosylation-like factor 6 interacting |
| | | protein 2 |
| 3.22481E−17 | AF084830 | KCNK5\|potassium channel, subfamily K, member 5 (TASK-2) |
| 4.56904E−17 | AF070614 | SCHIP1\|schwannomin interacting protein 1 |
| 4.93528E−17 | BF433019 | ESTs, Weakly similar to TRHY_HUMAN TRICHOHYALI [H. sapiens] |
| 5.54062E−17 | AA622986 | ESTs |
| 7.53411E−17 | NM_005401 | PTPN14\|protein tyrosine phosphatase, non-receptor type 14 |
| 8.78218E−17 | NM_002639 | SERPINB5\|serine (or cysteine) proteinase inhibitor, clade B (ovalbumin), |
| | | member 5 |
| 9.12461E−17 | U95089 | EGFR\|epidermal growth factor receptor (erythroblastic leukemia viral (v-erb- |
| | | b) oncogene homolog, avian) |
| 1.0631E−16 | NM_003034 | SIAT8A\|sialyltransferase 8A (alpha-N-acetylneuraminate: alpha-2,8- |
| | | sialytransferase, GD3 synthase) |
| 1.0631E−16 | AF308297 | PPP1R14C\|protein phosphatase 1, regulatory (inhibitor) subunit 14C |
| 2.02749E−16 | BC016004 | MARCO\|macrophage receptor with collagenous structure |
| 2.54298E−16 | AI741143 | Homo sapiens cDNA FLJ32401 fis, clone SKMUS2000339 |
| 3.06941E−16 | H29323 | SFRP1\|secreted frizzled-related protein 1 |
| 3.30861E−16 | AI188827 | PIM1\|pim-1 oncogene |
| 3.37105E−16 | AL110178 | TRIM2\|tripartite motif-containing 2 |
| 3.43538E−16 | AI740531 | MAPK4\|mitogen-activated protein kinase 4 |
| 6.01505E−16 | BC012107 | SH2D2A\|SH2 domain protein 2A |
| 6.4813E−16 | BC017918 | LOC64148\|17 kD fetal brain protein |
| 6.72616E−16 | AK026818 | Homo sapiens cDNA: FLJ23165 fis, clone LNG09846 |
| 7.24508E−16 | BC018646 | PLCG2\|phospholipase C, gamma 2 (phosphatidylinositol-specific) |

TABLE 9
50 gene sequences which define Subtype D

| P values | | |
|---|---|---|
| (Wilcoxon Test) | GeneID | Description |
| 2.77034E−09 | AA609183 | ESTs |
| 2.87559E−09 | AA843233 | ESTs, Weakly similar to I38344 titin, cardiac muscle [H. sapiens] |
| 1.15332E−08 | BF003134 | CLCA2\|chloride channel, calcium activated, family member 2 |
| 3.9503E−08 | BC017073 | Homo sapiens, Similar to RIKEN cDNA 1810054O13 gene, clone |
| | | IMAGE: 3845933, mRNA, partial cds |
| 4.23232E−08 | AL117406 | ABCC11\|ATP-binding cassette, sub-family C (CFTR/MRP), member 11 |
| 5.5684E−08 | BC005297 | KMO\|kynurenine 3-monooxygenase (kynurenine 3-hydroxylase) |
| 1.13109E−07 | BC002480 | FLJ13352\|hypothetical protein FLJ13352 |
| 1.73946E−07 | BC000051 | KIAA0950\|lifeguard |
| 1.79754E−07 | BC005246 | TM4SF3\|transmembrane 4 superfamily member 3 |
| 2.18736E−07 | AA991437 | ESTs |
| 2.65798E−07 | AW444437 | ESTs |
| 3.43985E−07 | AI090561 | M160\|scavenger receptor cysteine-rich type 1 protein M160 precursor |
| 4.03622E−07 | AI139456 | LOC118430\|small breast epithelial mucin |
| 4.73181E−07 | U63008 | HGD\|homogentisate 1,2-dioxygenase (homogentisate oxidase) |
| 5.36992E−07 | AI304573 | CEACAM7\|carcinoembryonic antigen-related cell adhesion molecule 7 |
| 6.09026E−07 | BC010910 | MCJ\|DNAJ domain-containing |
| 6.09026E−07 | NM_001197 | BIK\|BCL2-interacting killer (apoptosis-inducing) |
| 8.06728E−07 | X60069 | GGT1\|gamma-glutamyltransferase 1 |
| 9.13192E−07 | AK024899 | ENPP3\|ectonucleotide pyrophosphatase/phosphodiesterase 3 |
| 1.00177E−06 | BF508222 | ESTs |
| 1.28014E−06 | AL080207 | ABCA12\|ATP-binding cassette, sub-family A (ABC1), member 12 |
| 1.89723E−06 | AA913512 | LOC56624\|mitochondrial ceramidase |
| 2.01447E−06 | M30474 | GGT2\|gamma-glutamyltransferase 2 |
| 2.07567E−06 | AW666005 | PRM3\|protamine 3 |
| 2.27002E−06 | AI783781 | EST |
| 2.33874E−06 | NM_001445 | FABP6\|fatty acid binding protein 6, ileal (gastrotropin) |
| 2.55664E−06 | BC005257 | MSMB\|microseminoprotein, beta- |
| 2.96382E−06 | AK025757 | FLJ22104\|hypothetical protein FLJ22104 |
| 3.05238E−06 | BF511014 | CTRP2\|complement-c1q tumor necrosis factor-related protein 2 |
| 3.85783E−06 | AF027977 | PPEF1\|protein phosphatase, EF hand calcium-binding domain 1 |
| 3.97159E−06 | AK024360 | FLJ14298\|hypothetical protein FLJ14298 |
| 4.08891E−06 | X53578 | FUT3\|fucosyltransferase 3 (galactoside 3(4)-L-fucosyltransferase, Lewis |
| | | blood group included) |
| 5.61574E−06 | BC011020 | MPHOSPH6\|M-phase phosphoprotein 6 |
| 5.61574E−06 | AB014603 | KIAA0703\|KIAA0703 gene product |
| 6.11857E−06 | BC002805 | GJB1\|gap junction protein, beta 1, 32 kD (connexin 32, Charcot-Marie-Tooth |
| | | neuropathy, X-linked) |
| 6.47721E−06 | BI711505 | HLXB9\|homeo box HB9 |
| 6.47735E−06 | N51717 | ESTs |
| 6.85615E−06 | BC017772 | HT021\|HT021 |
| 7.4642E−06 | AF007149 | Homo sapiens clone 24771 mRNA sequence |
| 8.12347E−06 | AF331643 | Homo sapiens chromosome 17 open reading frame 26 (C17orf26) mRNA, |
| | | complete cds |
| 8.35512E−06 | H19129 | FGF12\|fibroblast growth factor 12 |
| 8.59342E−06 | AK025289 | KLHL2\|kelch-like 2, Mayven (Drosophila) |
| 8.83782E−06 | BC014209 | BM040\|uncharacterized bone marrow protein BM040 |
| 9.34702E−06 | BC011587 | Homo sapiens, Similar to RIKEN cDNA 1700018O18 gene, clone |
| | | IMAGE: 4121436, mRNA, partial cds |
| 9.61178E−06 | AW410306 | NXPH4\|neurexophilin 4 |
| 9.61219E−06 | BF108852 | ERBB2\|v-erb-b2 erythroblastic leukemia viral oncogene homolog 2, |
| | | neuro/glioblastoma derived oncogene homolog (avian) |
| 9.74693E−06 | BC016153 | Homo sapiens, Similar to hypothetical protein FLJ10134, clone MGC: 13208 |
| | | IMAGE: 3841102, mRNA, complete cds |
| 1.04507E−05 | AF023676 | TM7SF2\|transmembrane 7 superfamily member 2 |
| 1.07451E−05 | BC004925 | Homo sapiens, Similar to G protein-coupled receptor, family C, group 5, |
| | | member C, clone MGC: 10304 IMAGE: 3622005, mRNA, complete cds |
| 1.10479E−05 | AW299530 | ESTs |

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-7. (canceled)

8. A method to determine the prognosis or clinical course and aggressiveness of breast cancer of a subject comprising assaying for the expression level(s) of one or more genes in Table 2, 3, 4, 6, 7, 8, or 9 from a breast cancer cell sample from the subject.

9. The method of claim 8 wherein said assaying comprises preparing RNA, optionally labeled, from said sample and optionally converting said RNA into cDNA, optionally labeled.

10. The method of claim 9 wherein said RNA is not labeled and used for quantitative PCR.

11. The method of claim 9 wherein said assaying comprises using an array.

12. The method of claim 8 wherein said sample is a ductal lavage or fine needle aspiration or FFPE breast tissue sample.

13. The method of claim 12 wherein said sample is microdissected to isolate one or more cells that are breast cancer cells or suspected of being breast cancer cells.

14. The method of claim 10 wherein genes from Table 4 are used and further comprising determination of the ratio of the expression of an underexpressed gene to the expression of an overexpressed gene as an indicator of prognosis or clinical course and aggressiveness of breast cancer in said subject.

15. A method of determining prognosis of a subject having breast cancer, said method comprising:
assaying for the expression level(s) of one or more genes in Table 2, 3, 4, 6, 7, 8, or 9 from a breast cancer cell sample from said subject.

16. The method of claim 15 wherein said assaying comprises preparing RNA, optionally labeled, from said sample and optionally converting said RNA into cDNA, optionally labeled.

17. The method of claim 16 wherein said RNA is not labeled and used for quantitative PCR.

18. The method of claim 15 wherein said assaying comprises using an array.

19. The method of claim 15 wherein said sample is a ductal lavage or fine needle aspiration or FFPE breast tissue sample.

20. The method of claim 19 wherein said sample is microdissected to isolate one or more cells that are breast cancer cells or suspected of being breast cancer cells.

21. The method of claim 17 wherein genes from Table 4 are used and further comprising determination of the ratio of the expression of an underexpressed gene to the expression of an overexpressed gene as an indicator of prognosis in said subject.

22. A method to determine the survival outcome of a breast cancer afflicted subject comprising assaying a sample of breast cancer cells of said subject for the expression level(s) of one or more genes listed in Table 2, 3, 4, 6, 7, 8, or 9.

23. The method of claim 22 wherein said assaying comprises preparing RNA, optionally labeled, from said sample and optionally converting said RNA into cDNA, optionally labeled.

24. The method of claim 23 wherein said RNA is not labeled and used for quantitative PCR.

25. The method of claim 22 wherein said assaying comprises using an array.

26. The method of claim 22 wherein said sample is a ductal lavage or fine needle aspiration or FFPE breast tissue sample.

27. The method of claim 26 wherein said sample is microdissected to isolate one or more cells that are breast cancer cells or suspected of being breast cancer cells.

28. The method of claim 24 wherein genes from Table 4 are used and further comprising determination of the ratio of the expression of an underexpressed gene to the expression of an overexpressed gene as an indicator of prognosis in said subject.

29. (canceled)
